(19) United States
(12) Patent Application Publication
(10) Pub. No.: US 2016/0244436 A1
(43) Pub. Date: Aug. 25, 2016

(54) NOVEL FUNCTIONALIZED 5-(PHENOXYMETHYL)-1,3-DIOXANE ANALOGS EXHIBITING CYTOCHROME P450 INHIBITION AND THEIR METHOD OF USE

(71) Applicant: CORTENDO AB (PUBL), Radnor, PA (US)

(72) Inventors: Benjamin Eric Blass, Eagleville, PA (US); Magid A Abou-Gharbia, Exton, PA (US); Wayne E. Childers, New Hope, PA (US); Pravin Iyer, Bengaluru (IN); Joshodeep Boruwa, Mallapur (IN); Ramreddy Bobbala, Ramanthapur (IN); Rajashekar Reddy Nimmareddy, Uppal (IN)

(21) Appl. No.: 15/024,766

(22) PCT Filed: Sep. 25, 2014
(86) PCT No.: PCT/US14/57490

§ 371 Date: Mar. 24, 2016

Related U.S. Application Data
(60) Provisional application No. 61/882,625, filed on Sep. 25, 2013.

(51) Int. Cl. C07D 405/14; C07D 405/06

(52) U.S. Cl. CPC C07D 405/14; C07D 405/06

(57) ABSTRACT

Pharmaceutical compositions described in this document comprise 5-(phenoxymethyl)-1,3-dioxane analogs having a disease-modifying action in the treatment of diseases associated with the production of cortisol that include metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, cancer, stroke, incidentalomas, or any diseases involving the overproduction of cortisol.

### BRIEF SUMMARY

Embodiments described in this document are directed toward novel compounds of the formula (I),

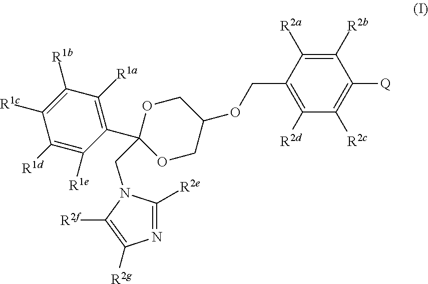

including hydrates, solvates, enantiomers, diastereomers, pharmaceutically acceptable salts, prodrugs and complexes thereof, wherein:

Q is selected from a group consisting of optionally substituted aryl, optionally substituted heteroaryl,

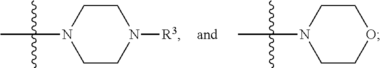

R^{1a}, R^{1b}, R^{1c}, R^{1d}, and R^{1e} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;

R^{2a}, R^{2b}, R^{2c}, R^{2d}, R^{2e}, R^{2f} and R^{2g} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆ optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;

R³ is selected from a group consisting of hydrogen, —SO₂R⁸, —C(O)NR⁹R¹⁰, C(O)R⁷—C(O)OR⁷,

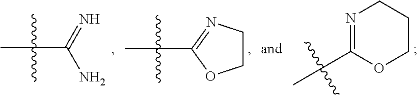

R^{4a} and R^{4b} are each independently selected from the group consisting of hydrogen, optionally substituted C₁₋₆ linear alkyl, optionally substituted C₁₋₆ branched alkyl, and optionally substituted C₃₋₇ cycloalkyl;

R⁵ is selected from the group consisting of hydrogen, optionally substituted C₁₋₆ linear alkyl, optionally substituted C₁₋₆ branched alkyl, and optionally substituted C₃₋₇ cycloalkyl;

R⁶ is selected from the group consisting of hydrogen, optionally substituted C₁₋₆ linear alkyl, optionally substituted C₁₋₆ branched alkyl, and optionally substituted C₃₋₇ cycloalkyl;

R⁷ is selected from the group consisting of optionally substituted C₁₋₆ linear alkyl, optionally substituted C₁₋₆ branched alkyl, and optionally substituted C₃₋₇ cycloalkyl;

R⁸ is selected from the group consisting of optionally substituted C₁₋₆ linear alkyl, optionally substituted C₁₋₆ branched alkyl, optionally substituted C₃₋₇ cycloalkyl, optionally substituted C₁₋₆ haloalkyl, optionally substituted aryl, optionally substituted heteroaryl, and optionally substituted C3-7 heterocyclyl;

R⁹ is selected from the group consisting of hydrogen, optionally substituted C₁₋₆ linear alkyl, optionally substituted C₁₋₆ branched alkyl, optionally substituted C₃₋₇ cycloalkyl, optionally substituted C₁₋₆ haloalkyl,

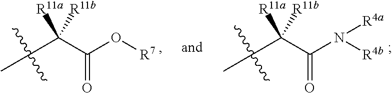

R¹⁰ is selected from the group consisting of hydrogen, optionally substituted C₁₋₆ linear alkyl, and optionally substituted C₁₋₆ branched alkyl;

R^{11a} and R^{11b} are each independently selected from the group consisting of hydrogen, optionally substituted C₁₋₆ linear alkyl, optionally substituted C₁₋₆ branched alkyl, optionally substituted aryl, optionally substituted benzyl, —CH₂OR⁶, and CH₂Heteroaryl.

Some embodiments described herein relate to a composition comprising an effective amount of at least one compounds according to the embodiments described in this document and at least one excipient.

Some embodiments relate to a method for treating, delaying, slowing, or inhibiting the progression of diseases that involve overproduction of cortisol, including, for example, metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas, said method comprising administering to a subject in need thereof an effective amount of a compound or composition according to embodiments, wherein the disease that involves overproduction of cortisol is treated, delayed, slowed, or inhibited.

Some embodiments yet further relate to a method for treating, delaying, slowing, or inhibiting the progression of diseases that involve overproduction of cortisol, including, for example, metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas, wherein said method comprises administering to a subject a composition comprising an effective amount of one or more compounds according to embodiments described herein and an excipient.

Some embodiments also relate to a method for treating, delaying, slowing, or inhibiting the progression of diseases or conditions associated with metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke, incidentalomas, or diseases that involve overproduction of cortisol. Said methods comprise administering to a subject an effective amount of a compound or composition according to embodiments described herein.

Some embodiments relate to a method of modulating cortisol activity, the method comprising administering to a subject in need thereof an effective amount of a compound or composition according to embodiments described herein, wherein the compound or composition modulates cortisol. In some embodiments, the compound or composition lowers cortisol levels in the subject.

Some embodiments yet further relate to a method for treating, delaying, slowing, or inhibiting the progression of disease or conditions associated with metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas and diseases that involve overproduction of cortisol, wherein said method comprises administering to a subject a composition comprising an effective amount of one or more compounds according to embodiments described herein and an excipient.

Some embodiments also relate to a method for treating, delaying, slowing, or inhibiting the progression of disease or conditions associated with overproduction of cortisol. Said methods comprise administering to a subject an effective amount of a compound or composition according to embodiments described herein.

Some embodiments yet further relate to a method for treating, delaying, slowing, or inhibiting the progression of disease or conditions associated with overproduction of cortisol, wherein said method comprises administering to a subject a composition comprising an effective amount of one or more compounds according to embodiments described herein and an excipient.

Some embodiments yet further relate to a method of lowering the concentration of cortisol in the circulatory system. Said methods comprise administering to a subject an effective amount of a compound or composition according to embodiments described herein.

Some embodiments yet further relate to a method of lowering the concentration of cortisol in the circulatory system, wherein said method comprises administering to a subject a composition comprising an effective amount of one or more compounds according to embodiments described herein and an excipient.

Some embodiments also relate to a method for treating, delaying, slowing, or inhibiting the progression of diseases that involve excess Cyp17 activity, including, for example, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject in need thereof an effective amount of a compound or composition according to embodiments, wherein the disease that involves excess Cyp17 activity is treated, delayed, slowed, or inhibited.

Some embodiments relate to a method for treating, delaying, slowing, or inhibiting the progression of diseases that involve excess Cyp17 activity, wherein said method comprises administering to a subject a composition comprising an effective amount of one or more compounds according to the embodiments described herein and an excipient.

Some embodiments also relate to a method for treating, delaying, slowing, or inhibiting the progression of diseases associated with Cyp17 activity, including, for example, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject in need thereof an effective amount of a compound or composition according to embodiments, wherein the Cyp17 activity is lowered, and wherein the disease that is associated with Cyp17 activity is treated, delayed, slowed, or inhibited.

Some embodiments relate to a method for treating, delaying, slowing, or inhibiting the progression of diseases associated with Cyp17 activity, said method comprising administering to a subject a composition comprising an effective amount of one or more compounds according to embodiments described herein and an excipient, wherein Cyp17 activity is lowered.

Some embodiments also relate to a method for lowering Cyp17 activity in a subject in need thereof, the method comprising administering to the subject an effective amount of a compound or composition according to embodiments, wherein the Cyp17 activity is lowered. In some embodiments, lowering of Cyp17 activity leads to a lowering of testosterone levels to castrate levels in the subject. In some embodiments, lowering of Cyp17 activity leads to a lowering of estrogen levels to post-menopausal levels in the subject. Some embodiments are directed to a method of treating cancer in a subject, the method comprising administering to the subject an effective amount of a compound or composition according to embodiments described herein, wherein Cyp17 activity is lowered. In some embodiments, Cyp17 activity is inhibited almost completely or completely. In some embodiments, lowering of Cyp17 activity leads to a lowering of testosterone levels to castrate levels in the subject. In some embodiments, lowering of Cyp17 activity leads to a lowering of estrogen levels to post-menopausal levels in the subject.

Some embodiments relate to a method of lowering Cyp17 activity, said method comprising administering to a subject a composition comprising an effective amount of one or more compounds according to embodiments described herein and an excipient.

Some embodiments relate to a method for treating, delaying, slowing, or inhibiting the progression of diseases that involve excess Cyp11B1 activity, including, for example, prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject in need thereof an effective amount of a compound or composition according to embodiments described herein, wherein the disease that involves excess Cyp11B1 activity is treated, delayed, slowed, or inhibited.

Some embodiments yet further relate to a method for treating, delaying, slowing, or inhibiting the progression of diseases that involve excess Cyp11B1 activity, including, for example, androgenic hormones and estrogens are involved, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, wherein said method comprises administering to a subject a composition comprising an effective amount of one or more compounds according to embodiments described herein and an excipient.

Some embodiments relate to a method for treating, delaying, slowing, or inhibiting the progression of diseases associated with Cyp11B1 activity, including, for example, prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject in need thereof an effective amount of a compound or composition according to embodiments described herein, wherein the Cyp11B activity is lowered and wherein the disease that involves excess Cyp11B1 activity is treated, delayed, slowed, or inhibited.

Some embodiments yet further relate to a method for treating, delaying, slowing, or inhibiting the progression of diseases associated with Cyp11B1 activity, including, for example, androgenic hormones and estrogens are involved, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject a composition comprising an effective amount of one or more compounds according to embodiments described herein and an excipient, wherein the Cyp11B1 activity is lowered.

Some embodiments also relate to a method for lowering Cyp11B1 activity in a subject in need thereof, the method comprising administering to the subject an effective amount of a compound or composition according to embodiments, wherein the Cyp17 activity is lowered. Some embodiments relate to a method of lowering Cyp11B1 activity, said method comprising administering to a subject a composition comprising an effective amount of one or more compounds according to embodiments described herein and an excipient.

Some embodiments relate to a method of inhibiting Cyp11B1 activity, said method comprising administering to a subject a composition comprising an effective amount of one or more compounds according to embodiments described herein and an excipient.

Some embodiments also relate to a method for treating, delaying, slowing, or inhibiting the progression of diseases that involve excess Cyp21 activity, including, for example, androgenic hormones and estrogens are involved, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject in need thereof an effective amount of a compound or composition according to embodiments described herein, wherein the disease that involves excess Cyp21 activity is treated, delayed, slowed, or inhibited.

Some embodiments yet further relate to a method for treating, delaying, slowing, or inhibiting the progression of diseases that involve excess Cyp21 activity, including, for example, androgenic hormones and estrogens are involved, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, wherein said method comprises administering to a subject a composition comprising an effective amount of one or more compounds according to embodiments described herein and an excipient.

Some embodiments also relate to a method for treating, delaying, slowing, or inhibiting the progression of diseases associated with Cyp21 activity, including, for example, androgenic hormones and estrogens are involved, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject in need thereof an effective amount of a compound or composition according to embodiments described herein, wherein Cyp21 activity is lowered, and wherein the disease that is associated with Cyp21 activity is treated, delayed, slowed, or inhibited.

Some embodiments yet further relate to a method for treating, delaying, slowing, or inhibiting the progression of diseases associated with Cyp21 activity, including, for example, androgenic hormones and estrogens are involved, such as prostate cancer, prostatic hypertrophy (prostatism), androgenic syndrome (masculinization), andromorphous baldness, breast cancer, mastopathy, uterine cancer, hirsutism, uterine fibroids, PCOS (polycystic ovarian syndrome), endometriosis, and ovarian cancer, said method comprising administering to a subject a composition comprising an effective amount of one or more compounds according to embodiments described herein and an excipient, wherein the Cyp21 activity is lowered.

Some embodiments also relate to a method for lowering Cyp21 activity in a subject in need thereof, the method comprising administering to the subject an effective amount of a compound or composition according to embodiments, wherein the Cyp17 activity is lowered. Some embodiments relate to a method of lowering Cyp21 activity, said method comprising administering to a subject a composition comprising an effective amount of one or more compounds according to embodiments described herein and an excipient.

Some embodiments relate to a method of inhibiting Cyp21 activity, said method comprising administering to a subject a composition comprising an effective amount of one or more compounds according to embodiments described herein and an excipient.

Some embodiments also relate to a method for lowering at least two of the following: Cyp17 activity, Cyp11B1 activity, and Cyp21 activity in a subject in need thereof, the method comprising administering to the subject an effective amount of a compound or composition according to embodiments described herein. In some embodiments, the method further modulates cortisol. Some embodiments relate to a method of treating, delaying, slowing, or inhibiting the progression of a disease selected from metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke, incidentalomas, related conditions, or a combination thereof, the method comprising administering to a subject in need thereof an effective amount of a compound or composition according to embodiments described herein, wherein the compound or composition lowers at least two of the following: Cyp17 activity, Cyp11B1 activity, and Cyp21 activity in the subject. In some embodiments, the compound or composition modulates cortisol. In some embodiments, the compound or composition lowers Cyp17 activity, Cyp11B1 activity, and Cyp21 activity in the subject.

Some embodiments further relate to a process for preparing the compounds of embodiments described herein.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to embodiments described herein.

### DETAILED DESCRIPTION

Embodiments of the present invention describe novel compounds useful for the treatment of diseases associated with the production of cortisol, such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke, incidentalomas, and related conditions. In some embodiments, the diseases that involve production of cortisol comprise diseases that involve an overproduction of cortisol. In some embodiments, diabetes mellitus includes diabetes mellitus type I, diabetes mellitus type II, prediabetes, latent autoimmune diabetes of adults (LADA), congenital diabetes, cystic fibrosis-related diabetes, steroid diabetes, monogenic diabetes, gestational diabetes, or a combination thereof.

Cortisol is a principal human glucocorticoid exhibiting many important physiological functions. It is involved in the regulation of the metabolism of proteins, carbohydrates, and fats; it counteracts insulin, maintains blood pressure and cardiovascular function, and suppresses the immune system's inflammatory response. However, pathological changes in adrenal and the upstream regulating switches can cause an overproduction of cortisol. One disease associated with overproduction of cortisol is metabolic syndrome. Over the course of the last three decades, a growing body of knowledge has been developed to describe metabolic syndrome, also referred to as “Syndrome X” or “Insulin Resistance Syndrome” (Reaven, G. M. Role of insulin resistance in human disease, Diabetes, 1988, 37, 1595-1607). Metabolic syndrome is defined as a cluster of abnormalities that occur in concert, including high blood pressure (BP), hyperglycemia, reduced high density lipoprotein cholesterol (HDL-C) levels, elevated triglycerides (TG) and abdominal obesity. The most widely accepted definition of this condition is based on the National Cholesterol Education Program (NCEP) Adult Treatment Panel-III (ATP-III), which provides for the diagnosis of metabolic syndrome in patients that meet at least three of parameters identified in table 1. Current estimates indicate that nearly 25% of the world's adult population suffers from metabolic syndrome, and the incidence is rising, largely as a result of increased obesity rates (Anagnostis, P.; Athyros, V. G.; Tziomalos, K.; Karagiannis, A.; Dimitri P. Mikhailidis, D. P. The Pathogenetic role of cortisol in the Metabolic Syndrome: A hypothesis, J. Clin. Endocrinol. Metab. 2009 94, 8, 2692-2701.).

TABLE 1
Metabolic Syndrome diagnostic parameters

| Parameter | Men | | Women | |
|---|---|---|---|---|
| Waist size | >102 | cm | >88 | cm |
| HDL-C | <40 | mg/dL | <50 | mg/dL |
| TG | >150 | mg/dL | >150 | mg/dL |
| BP | >130/85 | | >130/85 | |
| Fasting Glucose | >110 | mg/dL | >110 | mg/dL |

Cortisol production is regulated by several factors, including the enzymatic activity of the 11β-hydroxylase (Cyp11B1), 17α-hydroxylase-C17,20-lyase (Cyp17), and 21-hydroxylase (Cyp21). All three are members of the cytochrome P450 superfamily of enzymes. The 17α-hydroxylase/C₁₇₋₂₀ lyase enzyme complex is essential for the biosynthesis of androgens. CYP17 is a bifunctional enzyme which possess both a C₁₇₋₂₀-lyase activity and a C17-hydroxylase activity. These two alternative enzymatic activities of CYP17 result in the formation of critically different intermediates in steroid biosynthesis and each activity appear to be differentially and developmentally regulated.

Cyp11B1 catalyzes the final step of cortisol synthesis, hydroxylation of the C-11 position of deoxycortisol. Cyp17 has multiple functions in corticosteroid synthesis. The C-17 and C-20 positions of the steroid framework can be modified by this enzyme. Pregnenolone and progesterone are hydroxylated by Cyp17 at C-17 (hydroxylase activity), while the C-20/C-17 bond is cleaved by the same enzyme in 17-hydroxyprogesterone and 17-hydroxypregnenolone (lyase activity). Finally, Cyp21 catalyzes the hydroxylation of C-21 in steroids such as progesterone and 17α-hydroxy progesterone.

Compounds that inhibit the enzymatic activity of Cyp17, Cyp21, or Cyp11B1 will lead to a decrease in the synthesis of cortisol, which would treat, delay, slow, or inhibit the progression of diseases associated with the overproduction of cortisol such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas. Further, compounds that are dual inhibitors of Cyp17 and Cyp21 will lead to a decrease in the synthesis of cortisol, which would treat, delay, slow, or inhibit the progression of diseases associated with the overproduction of cortisol such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas. In addition, compounds that are dual inhibitors of Cyp17 and Cyp11B1 will lead to a decrease in the synthesis of cortisol, which would treat, delay, slow, or inhibit the progression of diseases associated with the overproduction of cortisol such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas. Further, compounds that are dual inhibitors of Cyp11B1 and Cyp21 will lead to a decrease in the synthesis of cortisol, which would treat, delay, slow, or inhibit the progression of diseases associated with the overproduction of cortisol such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas.

There is a long felt need for new treatments for diseases and symptoms associated with the overproduction of cortisol such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas, that are both disease-modifying and effective in treating patients. Embodiments of the present invention addresses the need to identify effective treatment for diseases and symptoms associated with the overproduction of cortisol, such as metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas.

The cortisol lowering agents of embodiments described herein are capable of treating, delaying, slowing, or inhibiting the progression of diseases associated with the overproduction of cortisol such as, for example, metabolic syndrome. It has been discovered that cortisol is a principal human glucocorticoid exhibiting many important physiological functions. It is involved in the regulation of the metabolism of proteins, carbohydrates, and fats; it counteracts insulin, maintains blood pressure and cardiovascular function, and suppresses the immune system's inflammatory response. However, pathological changes in adrenal gland or other tissues capable of secreting cortisol and the upstream regulating switches can cause an overproduction of cortisol. One disease associated with overproduction of cortisol is metabolic syndrome. In addition, the overproduction of cortisol is associated with hypertension, diabetes mellitus, obesity, headache, depression, Cushing's syndrome, pseudo-Cushing syndrome, cognitive impairment, dementia, heart failure, renal failure, psoriasis, glaucoma, cardiovascular disease, stroke and incidentalomas. Without wishing to be limited by theory, it is believed that cortisol lowering agents of embodiments described in this disclosure ameliorate, abate, otherwise cause to be controlled, diseases associated with the overproduction of cortisol, for example metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke and incidentalomas.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

As used herein, the term “consists of” or “consisting of” means that the method, use of formulation includes only the elements, steps, or ingredients specifically recited in the particular claimed embodiment or claim.

As used herein, the term “consisting essentially of” or “consists essentially of” means that the only active pharmaceutical ingredient in the formulation or method that treats the specified condition (e.g. Cushing's syndrome) is the specifically recited active pharmaceutical ingredient for treating the specified condition in the particular embodiment or claim; that is, the scope of the claim or embodiment is limited to the specified elements or steps and those that do not materially affect the basic and novel characteristic(s) of the particular embodiment or claimed invention.

In the application, where an element or component is said to be included in and/or selected from a list of recited elements or components, it should be understood that the element or component can be any one of the recited elements or components or a combination thereof, and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” means plus or minus 10% of the numerical value of the number with which it is being used. Therefore, about 50% means in the range of 45%-55%.

It should be understood that the order of steps or order for performing certain actions is immaterial so long as the present teachings remain operable. Moreover, two or more steps or actions can be conducted simultaneously

As used herein, the term “excess” refers to an amount or quantity surpassing what is considered normal or sufficient. For example, excess Cyp17 activity may refer to an above normal level of the C17-hydroxylase activity of CYP17 which promotes the overproduction of glucocorticoids or an above normal level of the C17,20-lyase activity of Cyp17 which promotes the overproduction of sex hormones. In some embodiments, excess Cyp17 activity may lead to overproduction of cortisol or an overproduction of androgenic or estrogenic hormones

As used herein, the term “halogen” includes chlorine, bromine, fluorine, iodine, or a combination thereof.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) refers independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tent-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Non-limiting examples of alkenyl groups include ethenyl, 3-propenyl, 1-propenyl (also 2-methylethenyl), isopropenyl (also 2-methylethen-2-yl), buten-4-yl, and the like. Non-limiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Non-limiting examples of alkynyl groups include ethynyl, prop-2-ynyl (also propargyl), propyn-1-yl, and 2-methyl-hex-4-yn-1-yl. Non-limiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. In some embodiments, cycloalkyl groups may be monocyclic (e.g., cyclohexyl) or polycyclic (e.g., containing fused, bridged, and/or Spiro ring systems), wherein the carbon atoms are located inside or outside of the ring system. Any suitable ring position of the cycloalkyl group can be covalently linked to the defined chemical structure. In some embodiments, cycloalkyl rings may be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “haloalkyl” may include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

As used herein, the term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

As used herein, the term “arylalkyl” or “aralkyl” refers to the group alkyl-aryl, where the alkyl and aryl groups are as defined herein. Aralkyl groups of embodiments described herein are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposes of embodiments described herein fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

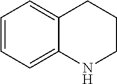

is, for the purposes of embodiments described herein, considered a heterocyclic unit. 6,7-Dihydro-5H-cyclopentapyrimidine having the formula:

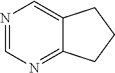

is, for the purposes of embodiments described herein, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

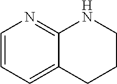

is, for the purposes of embodiments described herein, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR¹², —SR¹², —N(R¹²)₂, —NR¹²C(O)R¹², —SO₂R¹², —SO₂OR¹², —SO₂N(R¹²)₂, —C(O)R¹², —C(O)OR¹², —C(O)N(R¹²)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R¹²; wherein R¹², at each occurrence, independently is hydrogen, —OR¹³, —SR¹³, —C(O)R¹³, —C(O)OR¹³, —C(O)N(R¹³)₂, —SO₂R¹³, —S(O)₂OR¹³, —N(R¹³)₂, —NR¹³C(O)R¹³, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹² units taken together with the atom(s) to which they are bound form an optionally substituted carbocycle or heterocycle wherein said carbocycle or heterocycle has 3 to 7 ring atoms; wherein R¹³, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹³ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR¹⁴; for example, —OH, —OCH₃, —OCH₂CH₃, —OCH₂CH₂CH₃;
ii) —C(O)R¹⁴; for example, —COCH₃, —COCH₂CH₃, —COCH₂CH₂CH₃;
iii) —C(O)OR¹⁴; for example, —CO₂CH₃, —CO₂CH₂CH₃, —CO₂CH₂CH₂CH₃;
iv) —C(O)N(R¹⁴)₂; for example, —CONH₂, —CONHCH₃, —CON(CH₃)₂;
v) —N(R¹⁴)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹⁴; for example, —SO₂H; —SO₂CH₃; —SO₂C₆H₅;
ix) C₁-C₆ linear, branched, or cyclic alkyl;
x) Cyano
xi) Nitro;
xii) N(R¹⁴)C(O)R¹⁴;
xiii) Oxo (═O);
xiv) Heterocycle; and
xv) Heteroaryl.
   wherein each R¹⁴ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R¹⁴ units can be taken together to form a ring comprising 3-7 ring atoms. In certain aspects, each R¹⁴ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of embodiments described herein the terms “compound,” “analog,” and “composition of matter” stand equally well for the cortisol lowering agent described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R¹³)₂, each R¹³ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

A “therapeutically effective amount” or “effective amount” of a composition is a predetermined amount calculated to achieve the desired effect, i.e. treat, delay, slow, or inhibit the progression of diseases that involve overproduction of cortisol. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of embodiments described herein.

Embodiments described herein is directed toward novel compounds of the formula (I),

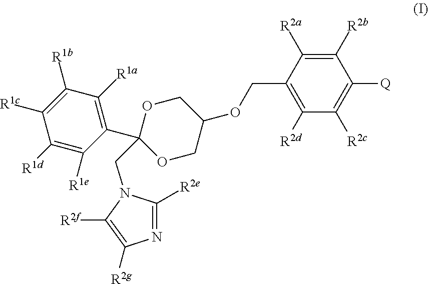

Including hydrates, solvates, enantiomers, diastereomers, pharmaceutically acceptable salts, prodrugs and complexes thereof, wherein:

Q is selected from a group consisting of optionally substituted aryl, optionally substituted heteroaryl,

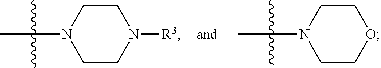

R^{1a}, R^{1b}, R^{1c}, R^{1d}, and R^{1e} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;

R^{2a}, R^{2b}, R^{2c}, R^{2d}, R^{2e}, R^{2f} and R^{2g} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆ optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;

R³ is selected from a group consisting of hydrogen, —SO₂R⁸, —C(O)NR⁹R¹⁰, —C(O)R⁷, —C(O)OR⁷,

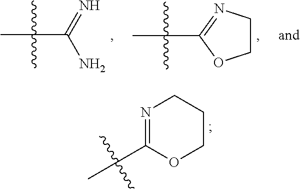

R^{4a} and R^{4b} are each independently selected from the group consisting of hydrogen, optionally substituted C₁₋₆ linear alkyl, optionally substituted C₁₋₆ branched alkyl, and optionally substituted C₃₋₇ cycloalkyl;

R⁵ is selected from the group consisting of hydrogen, optionally substituted C₁₋₆ linear alkyl, optionally substituted C₁₋₆ branched alkyl, and optionally substituted C₃₋₇ cycloalkyl;

R⁶ is selected from the group consisting of hydrogen, optionally substituted C₁₋₆ linear alkyl, optionally substituted C₁₋₆ branched alkyl, and optionally substituted C₃₋₇ cycloalkyl;

R⁷ is selected from the group consisting of optionally substituted C₁₋₆ linear alkyl, optionally substituted C₁₋₆ branched alkyl, and optionally substituted C₃₋₇ cycloalkyl;

R⁸ is selected from the group consisting of optionally substituted C₁₋₆ linear alkyl, optionally substituted C₁₋₆ branched alkyl, optionally substituted C₃₋₇ cycloalkyl, optionally substituted C₁₋₆ haloalkyl, optionally substituted aryl, optionally substituted heteroaryl, and optionally substituted C3-7 heterocyclyl;

R⁹ is selected from the group consisting of hydrogen, optionally substituted C₁₋₆ linear alkyl, optionally substituted C₁₋₆ branched alkyl, optionally substituted C₃₋₇ cycloalkyl, optionally substituted C₁₋₆ haloalkyl,

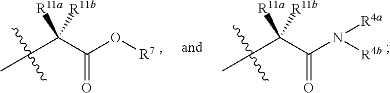

R¹⁰ is selected from the group consisting of hydrogen, optionally substituted C₁₋₆ linear alkyl, and optionally substituted C₁₋₆ branched alkyl;

R^{11a} and R^{11b} are each independently selected from the group consisting of hydrogen, optionally substituted C₁₋₆ linear alkyl, optionally substituted C₁₋₆ branched alkyl, optionally substituted aryl, optionally substituted benzyl, —CH₂OR⁶, and CH₂Heteroaryl.

Some embodiments include compounds having formula (II):

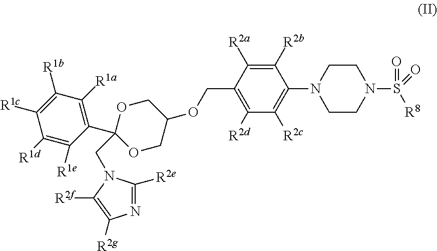

including hydrates, solvates, enantiomers, diastereomers, pharmaceutically acceptable salts, and complexes thereof.

Some embodiments include compounds having formula (III):

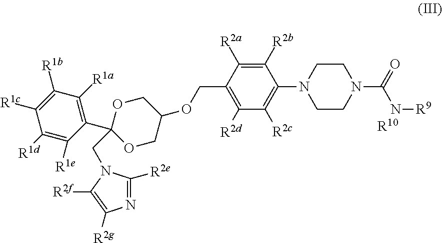

including hydrates, solvates, enantiomers, diastereomers pharmaceutically acceptable salts, and complexes thereof.

Some embodiments include compounds having formula (IV):

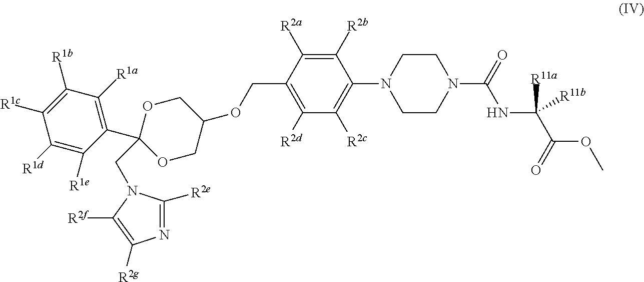

including hydrates, solvates, enantiomers, diastereomers, pharmaceutically acceptable salts, and complexes thereof.

Some embodiments include compounds having formula (V):

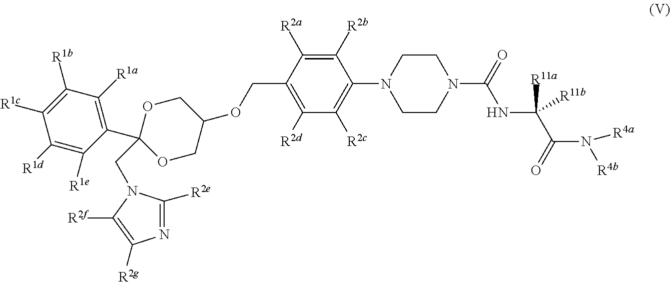

including hydrates, solvates, enantiomers, diastereomers, pharmaceutically acceptable salts, and complexes thereof.

Some embodiments include compounds having formula (VI):

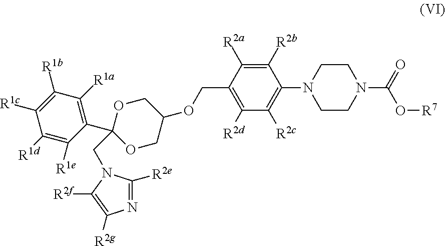

including hydrates, solvates, enantiomers, diastereomers, pharmaceutically acceptable salts, and complexes thereof.

Some embodiments include compounds having formula (VII):

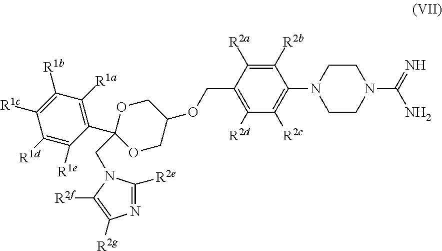

including hydrates, solvates, enantiomers, diastereomers, pharmaceutically acceptable salts, and complexes thereof.

Some embodiments include compounds having formula (VIII):

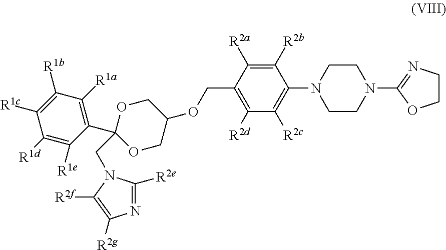

including hydrates, solvates, enantiomers, diastereomers, pharmaceutically acceptable salts, and complexes thereof.

The embodiments of the present invention include compounds having formula (IX):

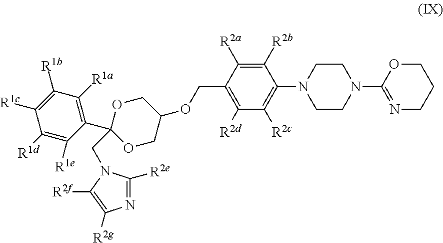

including hydrates, solvates, enantiomers, diastereomers, pharmaceutically acceptable salts, and complexes thereof.

Some embodiments include compounds having formula (X):

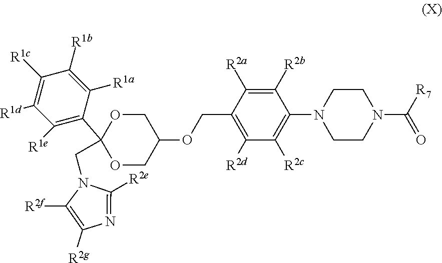

including hydrates, solvates, enantiomers, diastereomers, pharmaceutically acceptable salts, and complexes thereof.

Some embodiments include compounds having formula (Xa):

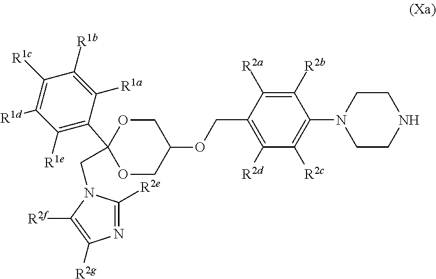

including hydrates, solvates, enantiomers, diastereomers, pharmaceutically acceptable salts, and complexes thereof.

In some embodiments Q is optionally substituted aryl.

In some embodiments Q is optionally substituted heteroaryl.

In some embodiments Q is

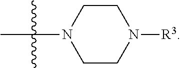

In some embodiments Q is

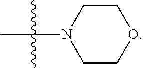 [SMILES: *N1CCOCC1]

In some embodiments R^{1a} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶.

In some embodiments R^{1b} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{1c} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{1d} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{1e} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{2a} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{2b} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{2c} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{ed} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{2e} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{2f} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵ COR⁶, —CO₂R⁶, —CO₂NR^{4a}R^{4b}, —NHSO₂R⁷, —SH, —SR⁷, —SO₂R⁷, and —SO₂NHR⁶.

In some embodiments R^{2g} of Formula I, II, III, IV, V, VI, VII, VIII, IX, X and Xa is selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, R^{2a} is —NR^{4a}R^{4b}, —NR⁵COR⁶, —CO₂R⁶, —CO₂NR^{4a}R^{4b}, —NHSO₂R⁷, —SH, —SR⁷, —SO₂R⁷, and —SO₂NHR⁶.

In some embodiments R³ of Formula I, II, III, IV, V, VI, VII, VIII, IX, X and Xa is selected from the group consisting of hydrogen, —SO₂R⁸, —C(O)NR⁹R¹⁰, —C(O)R⁷, —C(O)OR⁷,

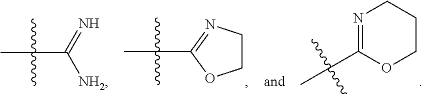

In some embodiments R^{4a} of Formula I, II, III, IV, V, VI, VII, VIII, IX, X and Xa is selected from the group consisting of hydrogen, optionally substituted C₁₋₆ linear alkyl, optionally substituted C₁₋₆ branched alkyl, and optionally substituted C₃₋₇ cycloalkyl.

In some embodiments R^{4b} of Formula I, II, III, IV, V, VI, VII, VIII, IX, X and Xa is selected from the group consisting of hydrogen, optionally substituted C₁₋₆ linear alkyl, optionally substituted C₁₋₆ branched alkyl, and optionally substituted C₃₋₇ cycloalkyl.

In some embodiments R⁵ of Formula I, II, III, IV, V, VI, VII, VIII, IX, X and Xa is selected from the group consisting of hydrogen, optionally substituted C₁₋₆ linear alky, optionally substituted C₁₋₆ branched alkyl, and optionally substituted C₃₋₇ cycloalkyl.

In some embodiments R⁶ of Formula I, II, III, IV, V, VI, VII, VIII, IX, X and Xa is selected from the group consisting of hydrogen, optionally substituted C₁₋₆ linear alkyl, optionally substituted C₁₋₆ branched alkyl, and optionally substituted C₃₋₇ cycloalkyl.

In some embodiments R⁷ of Formula I, II, III, IV, V, VI, VII, VIII, IX, X and Xa is selected from the group consisting of optionally substituted C₁₋₆ linear alkyl, optionally substituted C₁₋₆ branched alkyl, optionally substituted C₃₋₇ cycloalkyl.

In some embodiments R⁸ of Formula I, II, III, IV, V, VI, VII, VIII, IX, X and Xa is selected from the group consisting of optionally substituted C₁₋₆ linear alkyl, optionally substituted C₁₋₆ branched alkyl, optionally substituted C₃₋₇ cycloalkyl, optionally substituted C₁₋₆ haloalkyl, optionally substituted aryl, optionally substituted heteroaryl, and optionally substituted C₃₋₇heterocyclyl.

In some embodiments R⁹ of Formula I, II, III, IV, V, VI, VII, VIII, IX, X and Xa is selected from the group consisting of hydrogen, optionally substituted C₁₋₆ linear alkyl, optionally substituted C₁₋₆ branched alkyl, optionally substituted C₃₋₇ cycloalkyl, optionally substituted C₁₋₆ haloalkyl,

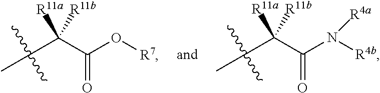

In some embodiments R¹⁰ of Formula I, II, III, IV, V, VI, VII, VIII, IX, X and Xa is selected from the group consisting of hydrogen, optionally substituted C₁₋₆ linear alkyl, and optionally substituted C₁₋₆ branched alkyl.

In some embodiments R^{11a} of Formula I, II, III, IV, V, VI, VII, VIII, IX, X and Xa is selected from the group consisting of hydrogen, optionally substituted C₁₋₆ linear alkyl, optionally substituted C₁₋₆ branched alkyl, optionally substituted aryl, optionally substituted benzyl, —CH₂OR⁶, and CH₂Heteroaryl.

In some embodiments R^{11b} of Formula I, II, III, IV, V, VI, VII, VIII, IX, X and Xa is selected from the group consisting of hydrogen, optionally substituted C₁₋₆ linear alkyl, optionally substituted C₁₋₆ branched alkyl, optionally substituted aryl, optionally substituted benzyl, —CH₂OR⁶, and CH₂Heteroaryl.

Exemplary embodiments include compounds having the formula (X) or a pharmaceutically acceptable salt form thereof:

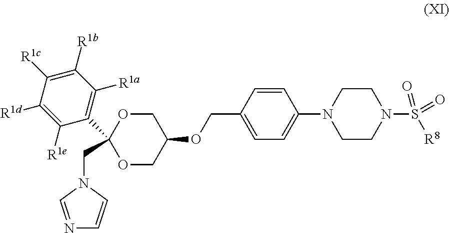

wherein non-limiting examples of R^{1a}, R^{1b}, R^{1c}, R^{1d}, R^{1e}, and R³ are defined herein below in Table 2.

TABLE 2

| Entry | R^{1a} | R^{1b} | R^{1c} | R^{1d} | R^{1e} | R⁸ |
|---|---|---|---|---|---|---|
| 1 | Cl | H | Cl | H | H | CH₃ |
| 2 | Cl | H | Cl | H | H | CH₂CH₃ |
| 3 | Cl | H | Cl | H | H | CH(CH₃)₂ |
| 4 | Cl | H | Cl | H | H | cyclopropyl |
| 5 | Cl | H | Cl | H | H | CH₂CH₂CH₂Cl |
| 6 | Cl | H | Cl | H | H | CH₂CF₃ |
| 7 | Cl | H | Cl | H | H | CF₃ |
| 8 | Cl | H | Cl | H | H | (CH₂)₂CH₃ |
| 9 | Cl | H | Cl | H | H | CH₂CH(CH₃)₂ |
| 10 | Cl | H | Cl | H | H | 3-cynanophenyl |
| 11 | Cl | H | Cl | H | H | 3-(trifluoromethoxy)phenyl |
| 12 | Cl | H | Cl | H | H | 3-pyridyl |
| 13 | Cl | H | Cl | H | H | 2-thiophene |
| 14 | Cl | H | Cl | H | H | 1-methylimidazol-2-yl |
| 15 | Cl | H | Cl | H | H | 1H-imidazol-4-yl |
| 16 | Cl | H | Cl | H | H | CH₂SO₂CH₃ |
| 17 | Cl | H | Cl | H | H | (CH₂)₂CF₃ |
| 18 | Cl | H | Cl | H | H | CF₂H |
| 19 | Cl | H | Cl | H | H | CH₂CF₂H |
| 20 | Cl | H | Cl | H | H | CH₂CN |
| 21 | Cl | H | Cl | H | H | (CH₂)₂OCH₃ |
| 22 | Cl | H | Cl | H | H | 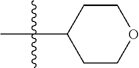 [SMILES: *C1CCOCC1] |
| 23 | Cl | H | H | H | H | CH₃ |
| 24 | Cl | H | H | H | H | CH₂CH₃ |
| 25 | Cl | H | H | H | H | CH(CH₃)₂ |
| 26 | Cl | H | H | H | H | cyclopropyl |
| 27 | Cl | H | H | H | H | CH₂CH₂CH₂Cl |
| 28 | Cl | H | H | H | H | CH₂CF₃ |
| 29 | Cl | H | H | H | H | CF₃ |
| 30 | Cl | H | H | H | H | (CH₂)₂CH₃ |
| 31 | Cl | H | H | H | H | CH₂CH(CH₃)₂ |
| 32 | Cl | H | H | H | H | 3-cynanophenyl |
| 33 | Cl | H | H | H | H | 3-(trifluoromethoxy)phenyl |
| 34 | Cl | H | H | H | H | 3-pyridyl |
| 35 | Cl | H | H | H | H | 2-thiophene |
| 36 | Cl | H | H | H | H | 1-methylimidazol-2-yl |
| 37 | Cl | H | H | H | H | 1H-imidazol-4-yl |
| 38 | Cl | H | H | H | H | CH₂SO₂CH₃ |
| 39 | Cl | H | H | H | H | (CH₂)₂CF₃ |
| 40 | Cl | H | H | H | H | CF₂H |
| 41 | Cl | H | H | H | H | CH₂CF₂H |
| 42 | Cl | H | H | H | H | CH₂CN |
| 43 | Cl | H | H | H | H | (CH₂)₂OCH₃ |
| 44 | Cl | H | H | H | H | 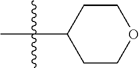 [SMILES: *C1CCOCC1] |

Exemplary embodiments include compounds having the formula (XII) or a pharmaceutically acceptable salt form thereof:

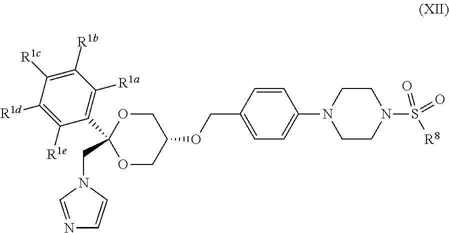

wherein non-limiting examples of R^{1a}, R^{1b}, R^{1c}, R^{1d}, R^{1e}, and R³ are defined herein below in Table 3.

TABLE 3

| Entry | R^{1a} | R^{1b} | R^{1c} | R^{1d} | R^{1e} | R⁸ |
|---|---|---|---|---|---|---|
| 1 | Cl | H | Cl | H | H | CH₃ |
| 2 | Cl | H | Cl | H | H | CH₂CH₃ |
| 3 | Cl | H | Cl | H | H | CH(CH₃)₂ |
| 4 | Cl | H | Cl | H | H | cyclopropyl |
| 5 | Cl | H | Cl | H | H | CH₂CH₂CH₂Cl |
| 6 | Cl | H | Cl | H | H | CH₂CF₃ |
| 7 | Cl | H | Cl | H | H | CF₃ |
| 8 | Cl | H | Cl | H | H | (CH₂)₂CH₃ |
| 9 | Cl | H | Cl | H | H | CH₂CH(CH₃)₂ |
| 10 | Cl | H | Cl | H | H | 3-cynanophenyl |
| 11 | Cl | H | Cl | H | H | 3-(trifluoromethoxy)phenyl |
| 12 | Cl | H | Cl | H | H | 3-pyridyl |
| 13 | Cl | H | Cl | H | H | 2-thiophene |
| 14 | Cl | H | Cl | H | H | 1-methylimidazol-2-yl |
| 15 | Cl | H | Cl | H | H | 1H-imidazol-4-yl |
| 16 | Cl | H | Cl | H | H | CH₂SO₂CH₃ |
| 17 | Cl | H | Cl | H | H | (CH₂)₂CF₃ |
| 18 | Cl | H | Cl | H | H | CF₂H |
| 19 | Cl | H | Cl | H | H | CH₂CF₂H |
| 20 | Cl | H | Cl | H | H | CH₂CN |
| 21 | Cl | H | Cl | H | H | (CH₂)₂OCH₃ |
| 22 | Cl | H | Cl | H | H | 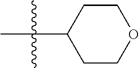 [SMILES: *C1CCOCC1] |
| 23 | Cl | H | H | H | H | CH₃ |
| 24 | Cl | H | H | H | H | CH₂CH₃ |
| 25 | Cl | H | H | H | H | CH(CH₃)₂ |
| 26 | Cl | H | H | H | H | cyclopropyl |
| 27 | Cl | H | H | H | H | CH₂CH₂CH₂Cl |
| 28 | Cl | H | H | H | H | CH₂CF₃ |
| 29 | Cl | H | H | H | H | CF₃ |
| 30 | Cl | H | H | H | H | (CH₂)₂CH₃ |
| 31 | Cl | H | H | H | H | CH₂CH(CH₃)₂ |
| 32 | Cl | H | H | H | H | 3-cynanophenyl |
| 33 | Cl | H | H | H | H | 3-(trifluoromethoxy)phenyl |
| 34 | Cl | H | H | H | H | 3-pyridyl |
| 35 | Cl | H | H | H | H | 2-thiophene |
| 36 | Cl | H | H | H | H | 1-methylimidazol-2-yl |
| 37 | Cl | H | H | H | H | 1H-imidazol-4-yl |
| 38 | Cl | H | H | H | H | CH₂SO₂CH₃ |
| 39 | Cl | H | H | H | H | (CH₂)₂CF₃ |
| 40 | Cl | H | H | H | H | CF₂H |
| 41 | Cl | H | H | H | H | CH₂CF₂H |
| 42 | Cl | H | H | H | H | CH₂CN |
| 43 | Cl | H | H | H | H | (CH₂)₂OCH₃ |
| 44 | Cl | H | H | H | H | 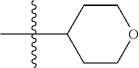 [SMILES: *C1CCOCC1] |

Exemplary embodiments include compounds having the formula (XIII) or a pharmaceutically acceptable salt form thereof:

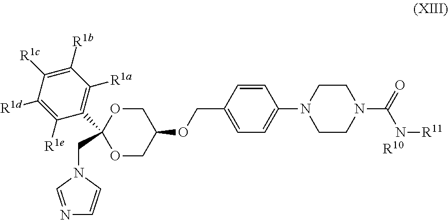

wherein non-limiting examples of R^{1a}, R^{1b}, R^{1c}, R^{1d}, R^{1e}, R⁹, and R¹⁰ are defined herein below in Table 4.

TABLE 4

| Entry | R^{1a} | R^{1b} | R^{1c} | R^{1d} | R^{1e} | R⁹ | R¹⁰ |
|---|---|---|---|---|---|---|---|
| 1 | Cl | H | Cl | H | H | H | CH₃ |
| 2 | Cl | H | Cl | H | H | H | CH₂CH₃ |
| 3 | Cl | H | Cl | H | H | H | CH(CH₃)₂ |
| 4 | Cl | H | Cl | H | H | H | cyclopropyl |
| 5 | Cl | H | Cl | H | H | CH₃ | CH₃ |
| 6 | Cl | H | Cl | H | H | CH₂CH₃ | CH₂CH₃ |
| 7 | Cl | H | Cl | H | H | CH(CH₃)₂ | CH(CH₃)₂ |
| 8 | Cl | H | Cl | H | H | cyclopropyl | cyclopropyl |
| 9 | Cl | H | H | H | H | H | CH₃ |
| 10 | Cl | H | H | H | H | H | CH₂CH₃ |
| 11 | Cl | H | H | H | H | H | CH(CH₃)₂ |
| 12 | Cl | H | H | H | H | H | cyclopropyl |
| 13 | Cl | H | H | H | H | CH₃ | CH₃ |
| 14 | Cl | H | H | H | H | CH₂CH₃ | CH₂CH₃ |
| 15 | Cl | H | H | H | H | CH(CH₃)₂ | CH(CH₃)₂ |
| 16 | Cl | H | H | H | H | cyclopropyl | cyclopropyl |

Exemplary embodiments include compounds having the formula (XIV) or a pharmaceutically acceptable salt form thereof:

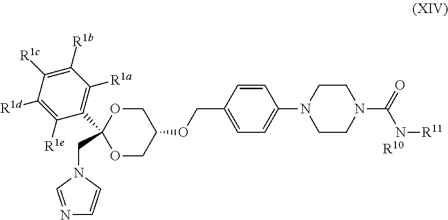

wherein non-limiting examples of R^{1a}, R^{1b}, R^{1c}, R^{1d}, R^{1e}, R⁹, and R¹⁰ are defined herein below in Table 5.

TABLE 5

| Entry | R^{1a} | R^{1b} | R^{1c} | R^{1d} | R^{1e} | R⁹ | R¹⁰ |
|---|---|---|---|---|---|---|---|
| 1 | Cl | H | Cl | H | H | H | CH₃ |
| 2 | Cl | H | Cl | H | H | H | CH₂CH₃ |
| 3 | Cl | H | Cl | H | H | H | CH(CH₃)₂ |
| 4 | Cl | H | Cl | H | H | H | cyclopropyl |
| 5 | Cl | H | Cl | H | H | CH₃ | CH₃ |
| 6 | Cl | H | Cl | H | H | CH₂CH₃ | CH₂CH₃ |
| 7 | Cl | H | Cl | H | H | CH(CH₃)₂ | CH(CH₃)₂ |
| 8 | Cl | H | Cl | H | H | cyclopropyl | cyclopropyl |
| 9 | Cl | H | H | H | H | H | CH₃ |
| 10 | Cl | H | H | H | H | H | CH₂CH₃ |
| 11 | Cl | H | H | H | H | H | CH(CH₃)₂ |
| 12 | Cl | H | H | H | H | H | cyclopropyl |
| 13 | Cl | H | H | H | H | CH₃ | CH₃ |
| 14 | Cl | H | H | H | H | CH₂CH₃ | CH₂CH₃ |
| 15 | Cl | H | H | H | H | CH(CH₃)₂ | CH(CH₃)₂ |
| 16 | Cl | H | H | H | H | cyclopropyl | cyclopropyl |

Exemplary embodiments include compounds having the formula (XV) or a pharmaceutically acceptable salt form thereof:

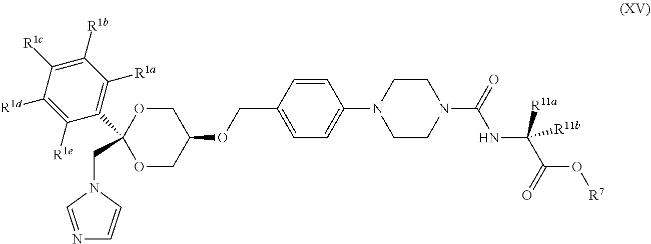

wherein non-limiting examples of R^{1a}, R^{1b}, R^{1c}, R^{1d}, R^{1e}, R^{11a}, R^{11b}, and R⁷ are defined herein below in Table 6.

TABLE 6

| Entry | R^{1a} | R^{1b} | R^{1c} | R^{1d} | R^{1e} | R^{11a} | R^{11b} | R⁷ |
|---|---|---|---|---|---|---|---|---|
| 1 | Cl | H | Cl | H | H | H | H | CH₃ |
| 2 | Cl | H | Cl | H | H | H | H | CH₂CH₃ |
| 3 | Cl | H | Cl | H | H | CH₃ | H | CH₃ |
| 4 | Cl | H | Cl | H | H | CH₃ | H | CH₂CH₃ |
| 5 | Cl | H | Cl | H | H | H | CH₃ | CH₃ |
| 6 | Cl | H | Cl | H | H | H | CH₃ | CH₂CH₃ |
| 7 | Cl | H | Cl | H | H | CH(CH₃)₂ | H | CH₃ |
| 8 | Cl | H | Cl | H | H | CH(CH₃)₂ | H | CH₂CH₃ |
| 9 | Cl | H | Cl | H | H | H | CH(CH₃)₂ | CH₃ |
| 10 | Cl | H | Cl | H | H | H | CH(CH₃)₂ | CH₂CH₃ |
| 11 | Cl | H | Cl | H | H | CH₂Ph | H | CH₃ |
| 12 | Cl | H | Cl | H | H | CH₂Ph | H | CH₂CH₃ |
| 13 | Cl | H | Cl | H | H | H | CH₂Ph | CH₃ |
| 14 | Cl | H | Cl | H | H | H | CH₂Ph | CH₂CH₃ |
| 15 | Cl | H | H | H | H | H | H | CH₃ |
| 16 | Cl | H | H | H | H | H | H | CH₂CH₃ |
| 17 | Cl | H | H | H | H | CH₃ | H | CH₃ |
| 18 | Cl | H | H | H | H | CH₃ | H | CH₂CH₃ |
| 19 | Cl | H | H | H | H | H | CH₃ | CH₃ |
| 20 | Cl | H | H | H | H | H | CH₃ | CH₂CH₃ |
| 21 | Cl | H | H | H | H | CH(CH₃)₂ | H | CH₃ |
| 22 | Cl | H | H | H | H | CH(CH₃)₂ | H | CH₂CH₃ |
| 23 | Cl | H | H | H | H | H | CH(CH₃)₂ | CH₃ |
| 24 | Cl | H | H | H | H | H | CH(CH₃)₂ | CH₂CH₃ |
| 25 | Cl | H | H | H | H | CH₂Ph | H | CH₃ |
| 26 | Cl | H | H | H | H | CH₂Ph | H | CH₂CH₃ |
| 27 | Cl | H | H | H | H | H | CH₂Ph | CH₃ |
| 28 | Cl | H | H | H | H | H | CH₂Ph | CH₂CH₃ |

Exemplary embodiments include compounds having the formula (XVI) or a pharmaceutically acceptable salt form thereof:

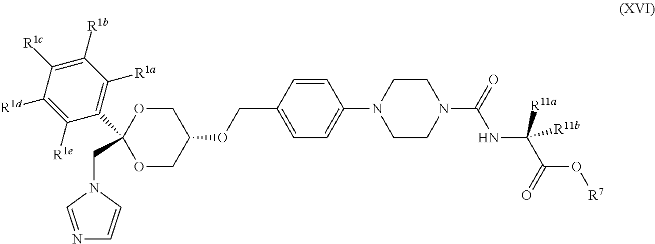

wherein non-limiting examples of R^{1a}, R^{1b}, R^{1c}, R^{1d}, R^{1e}, R^{11a}, R^{11b}, and R⁷ are defined herein below in Table 7.

TABLE 7

| Entry | R^{1a} | R^{1b} | R^{1c} | R^{1d} | R^{1e} | R^{11a} | R^{11b} | R⁷ |
|---|---|---|---|---|---|---|---|---|
| 1 | Cl | H | Cl | H | H | H | H | CH₃ |
| 2 | Cl | H | Cl | H | H | H | H | CH₂CH₃ |
| 3 | Cl | H | Cl | H | H | CH₃ | H | CH₃ |
| 4 | Cl | H | Cl | H | H | CH₃ | H | CH₂CH₃ |
| 5 | Cl | H | Cl | H | H | H | CH₃ | CH₃ |
| 6 | Cl | H | Cl | H | H | H | CH₃ | CH₂CH₃ |
| 7 | Cl | H | Cl | H | H | CH(CH₃)₂ | H | CH₃ |
| 8 | Cl | H | Cl | H | H | CH(CH₃)₂ | H | CH₂CH₃ |
| 9 | Cl | H | Cl | H | H | H | CH(CH₃)₂ | CH₃ |
| 10 | Cl | H | Cl | H | H | H | CH(CH₃)₂ | CH₂CH₃ |
| 11 | Cl | H | Cl | H | H | CH₂Ph | H | CH₃ |
| 12 | Cl | H | Cl | H | H | CH₂Ph | H | CH₂CH₃ |
| 13 | Cl | H | Cl | H | H | H | CH₂Ph | CH₃ |
| 14 | Cl | H | Cl | H | H | H | CH₂Ph | CH₂CH₃ |
| 15 | Cl | H | H | H | H | H | H | CH₃ |
| 16 | Cl | H | H | H | H | H | H | CH₂CH₃ |
| 17 | Cl | H | H | H | H | CH₃ | H | CH₃ |
| 18 | Cl | H | H | H | H | CH₃ | H | CH₂CH₃ |
| 19 | Cl | H | H | H | H | H | CH₃ | CH₃ |
| 20 | Cl | H | H | H | H | H | CH₃ | CH₂CH₃ |
| 21 | Cl | H | H | H | H | CH(CH₃)₂ | H | CH₃ |
| 22 | Cl | H | H | H | H | CH(CH₃)₂ | H | CH₂CH₃ |
| 23 | C1 | H | H | H | H | H | CH(CH₃)₂ | CH₃ |
| 24 | C1 | H | H | H | H | H | CH(CH₃)₂ | CH₂CH₃ |
| 25 | C1 | H | H | H | H | CH₂Ph | H | CH₃ |
| 26 | C1 | H | H | H | H | CH₂Ph | H | CH₂CH₃ |
| 27 | C1 | H | H | H | H | H | CH₂Ph | CH₃ |
| 28 | C1 | H | H | H | H | H | CH₂Ph | CH₂CH₃ |

Exemplary embodiments include compounds having the formula (XVII) or a pharmaceutically acceptable salt form thereof:

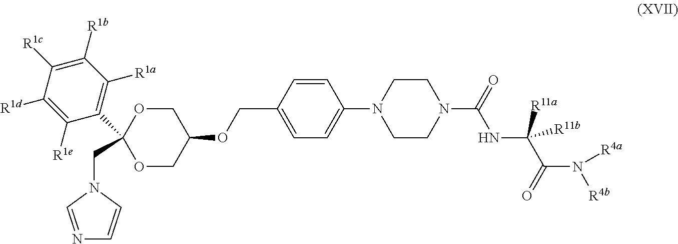

wherein non-limiting examples of R^{1a}, R^{1b}, R^{1c}, R^{1d}, R^{1e}, R^{11a}, R^{11b}, R^{4a}, and R^{4b} are defined herein below in Table 8.

TABLE 8

| Entry | R^{1a} | R^{1b} | R^{1c} | R^{1d} | R^{1e} | R^{11a} | R^{11b} | R^{4a} | R^{4b} |
|---|---|---|---|---|---|---|---|---|---|
| 1 | Cl | H | Cl | H | H | H | H | CH₃ | H |
| 2 | Cl | H | Cl | H | H | H | H | CH₂CH₃ | H |
| 3 | Cl | H | Cl | H | H | CH₃ | H | CH₃ | H |
| 4 | Cl | H | Cl | H | H | CH₃ | H | CH₂CH₃ | H |
| 5 | Cl | H | Cl | H | H | H | CH₃ | CH₃ | H |
| 6 | Cl | H | Cl | H | H | H | CH₃ | CH₂CH₃ | H |
| 7 | Cl | H | Cl | H | H | CH(CH₃)₂ | H | CH₃ | H |
| 8 | Cl | H | Cl | H | H | CH(CH₃)₂ | H | CH₂CH₃ | H |
| 9 | Cl | H | Cl | H | H | H | CH(CH₃)₂ | CH₃ | H |
| 10 | Cl | H | Cl | H | H | H | CH(CH₃)₂ | CH₂CH₃ | H |
| 11 | Cl | H | Cl | H | H | CH₂Ph | H | CH₃ | H |
| 12 | Cl | H | Cl | H | H | CH₂Ph | H | CH₂CH₃ | H |
| 13 | Cl | H | Cl | H | H | H | CH₂Ph | CH₃ | H |
| 14 | Cl | H | Cl | H | H | H | CH₂Ph | CH₂CH₃ | H |
| 15 | Cl | H | H | H | H | H | H | CH₃ | H |
| 16 | Cl | H | H | H | H | H | H | CH₂CH₃ | H |
| 17 | Cl | H | H | H | H | CH₃ | H | CH₃ | H |
| 18 | Cl | H | H | H | H | CH₃ | H | CH₂CH₃ | H |
| 19 | Cl | H | H | H | H | H | CH₃ | CH₃ | H |
| 20 | Cl | H | H | H | H | H | CH₃ | CH₂CH₃ | H |
| 21 | Cl | H | H | H | H | CH(CH₃)₂ | H | CH₃ | H |
| 22 | Cl | H | H | H | H | CH(CH₃)₂ | H | CH₂CH₃ | H |
| 23 | Cl | H | H | H | H | H | CH(CH₃)₂ | CH₃ | H |
| 24 | Cl | H | H | H | H | H | CH(CH₃)₂ | CH₂CH₃ | H |
| 25 | Cl | H | H | H | H | CH₂Ph | H | CH₃ | H |
| 26 | Cl | H | H | H | H | CH₂Ph | H | CH₂CH₃ | H |
| 27 | Cl | H | H | H | H | H | CH₂Ph | CH₃ | H |
| 28 | Cl | H | H | H | H | H | CH₂Ph | CH₂CH₃ | H |

Exemplary embodiments include compounds having the formula (XVIII) or a pharmaceutically acceptable salt form thereof:

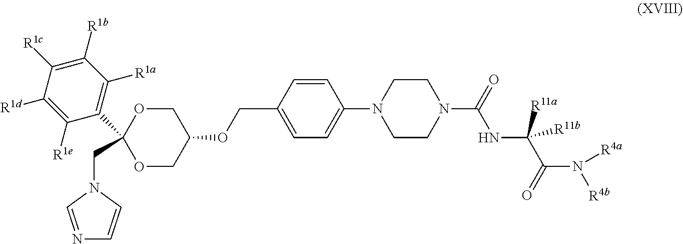

wherein non-limiting examples of R^{1a}, R^{1b}, R^{1c}, R^{1d}, R^{1e}, R^{11a}, R^{11b}, R^{4a}, and R^{4b} are defined herein below in Table 9.

TABLE 9

| Entry | R^{1a} | R^{1b} | R^{1c} | R^{1d} | R^{1e} | R^{11a} | R^{11b} | R^{4a} | R^{4b} |
|---|---|---|---|---|---|---|---|---|---|
| 1 | Cl | H | Cl | H | H | H | H | CH₃ | H |
| 2 | Cl | H | Cl | H | H | H | H | CH₂CH₃ | H |
| 3 | Cl | H | Cl | H | H | CH₃ | H | CH₃ | H |
| 4 | Cl | H | Cl | H | H | CH₃ | H | CH₂CH₃ | H |
| 5 | Cl | H | Cl | H | H | H | CH₃ | CH₃ | H |
| 6 | Cl | H | Cl | H | H | H | CH₃ | CH₂CH₃ | H |
| 7 | Cl | H | Cl | H | H | CH(CH₃)₂ | H | CH₃ | H |
| 8 | Cl | H | Cl | H | H | CH(CH₃)₂ | H | CH₂CH₃ | H |
| 9 | Cl | H | Cl | H | H | H | CH(CH₃)₂ | CH₃ | H |
| 10 | Cl | H | Cl | H | H | H | CH(CH₃)₂ | CH₂CH₃ | H |
| 11 | Cl | H | Cl | H | H | CH₂Ph | H | CH₃ | H |
| 12 | Cl | H | Cl | H | H | CH₂Ph | H | CH₂CH₃ | H |
| 13 | Cl | H | Cl | H | H | H | CH₂Ph | CH₃ | H |
| 14 | Cl | H | Cl | H | H | H | CH₂Ph | CH₂CH₃ | H |
| 15 | Cl | H | H | H | H | H | H | CH₃ | H |
| 16 | Cl | H | H | H | H | H | H | CH₂CH₃ | H |
| 17 | Cl | H | H | H | H | CH₃ | H | CH₃ | H |
| 18 | Cl | H | H | H | H | CH₃ | H | CH₂CH₃ | H |
| 19 | Cl | H | H | H | H | H | CH₃ | CH₃ | H |
| 20 | Cl | H | H | H | H | H | CH₃ | CH₂CH₃ | H |
| 21 | Cl | H | H | H | H | CH(CH₃)₂ | H | CH₃ | H |
| 22 | Cl | H | H | H | H | CH(CH₃)₂ | H | CH₂CH₃ | H |
| 23 | Cl | H | H | H | H | H | CH(CH₃)₂ | CH₃ | H |
| 24 | Cl | H | H | H | H | H | CH(CH₃)₂ | CH₂CH₃ | H |
| 25 | Cl | H | H | H | H | CH₂Ph | H | CH₃ | H |
| 26 | Cl | H | H | H | H | CH₂Ph | H | CH₂CH₃ | H |
| 27 | Cl | H | H | H | H | H | CH₂Ph | CH₃ | H |
| 28 | Cl | H | H | H | H | H | CH₂Ph | CH₂CH₃ | H |

Exemplary embodiments include compounds having the formula (XIX) or a pharmaceutically acceptable salt form thereof:

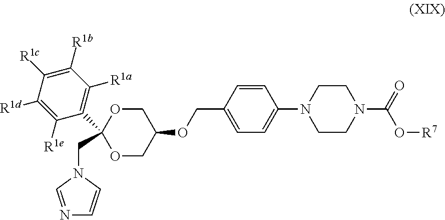

wherein non-limiting examples of R^{1a}, R^{1b}, R^{1c}, R^{1d}, R^{1e}, and R⁷ are defined herein below in Table 10.

TABLE 10

| Entry | R^{1a} | R^{1b} | R^{1c} | R^{1d} | R^{1e} | R⁷ |
|---|---|---|---|---|---|---|
| 1 | Cl | H | Cl | H | H | CH₃ |
| 2 | Cl | H | Cl | H | H | CH₂CH₃ |
| 3 | Cl | H | Cl | H | H | CH(CH₃)₂ |
| 4 | Cl | H | Cl | H | H | Cyclopropyl |
| 5 | Cl | H | Cl | H | H | (CH₂)₂OCH₃ |
| 6 | Cl | H | H | H | H | CH₃ |
| 7 | Cl | H | H | H | H | CH₂CH₃ |
| 9 | Cl | H | H | H | H | CH(CH₃)₂ |
| 10 | Cl | H | H | H | H | Cyclopropyl |
| 11 | Cl | H | H | H | H | (CH₂)₂OCH₃ |

Exemplary embodiments include compounds having the formula (XX) or a pharmaceutically acceptable salt form thereof:

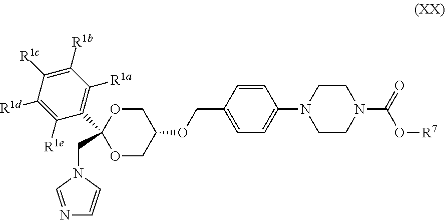

wherein non-limiting examples of R^{1a}, R^{1b}, R^{1c}, R^{1d}, R^{1e}, and R⁷ are defined herein below in Table 11.

TABLE 11

| Entry | R^{1a} | R^{1b} | R^{1c} | R^{1d} | R^{1e} | R⁷ |
|---|---|---|---|---|---|---|
| 1 | Cl | H | Cl | H | H | CH₃ |
| 2 | Cl | H | Cl | H | H | CH₂CH₃ |
| 3 | Cl | H | Cl | H | H | CH(CH₃)₂ |
| 4 | Cl | H | Cl | H | H | Cyclopropyl |
| 5 | Cl | H | Cl | H | H | (CH₂)₂OCH₃ |
| 6 | Cl | H | H | H | H | CH₃ |
| 7 | Cl | H | H | H | H | CH₂CH₃ |
| 9 | Cl | H | H | H | H | CH(CH₃)₂ |
| 10 | Cl | H | H | H | H | Cyclopropyl |
| 11 | Cl | H | H | H | H | (CH₂)₂OCH₃ |

Exemplary embodiments include compounds having the formula (XXI) or a pharmaceutically acceptable salt form thereof:

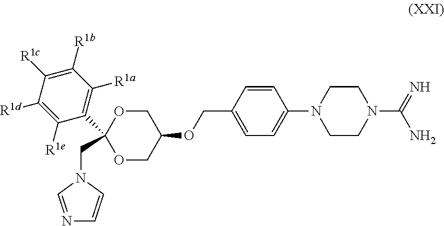

wherein non-limiting examples of R^{1a}, R^{1b}, R^{1c}, R^{1d}, and R^{1e} are defined herein below in Table 12.

TABLE 12

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXII) or a pharmaceutically acceptable salt form thereof:

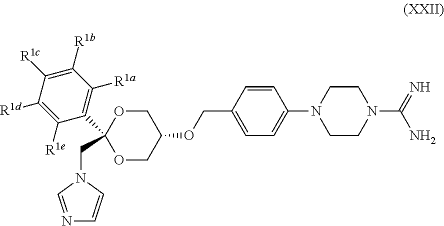

wherein non-limiting examples of R^{1a}, R^{1b}, R^{1c}, R^{1d}, and R^{1e} are defined herein below in Table 13.

TABLE 13

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXIII) or a pharmaceutically acceptable salt form thereof:

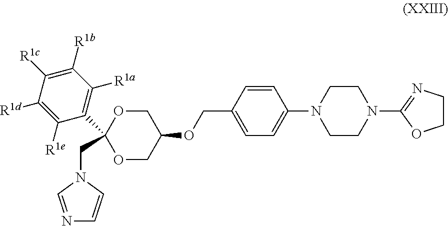

wherein non-limiting examples of R^{1a}, R^{1b}, R^{1c}, R^{1d}, and R^{1e} are defined herein below in Table 14.

TABLE 14

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXIV) or a pharmaceutically acceptable salt form thereof:

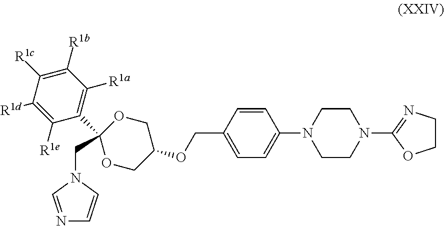

wherein non-limiting examples of R^{1a}, R^{1b}, R^{1c}, R^{1d}, and R^{1e} are defined herein below in Table 15.

TABLE 15

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXV) or a pharmaceutically acceptable salt form thereof:

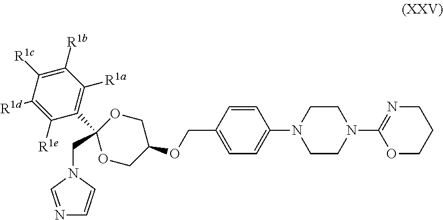

wherein non-limiting examples of R^{1a}, R^{1b}, R^{1c}, R^{1d}, and R^{1e} are defined herein below in Table 16.

TABLE 16

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXVI) or a pharmaceutically acceptable salt form thereof:

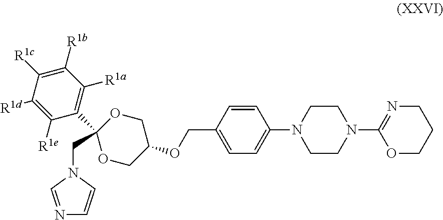

wherein non-limiting examples of R^{1a}, R^{1b}, R^{1c}, R^{1d}, and R^{1e} are defined herein below in Table 17.

TABLE 17

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXVII) or a pharmaceutically acceptable salt form thereof:

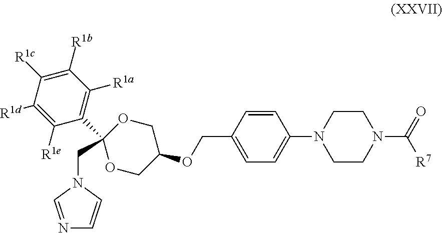

wherein non-limiting examples of R^{1a}, R^{1b}, R^{1c}, R^{1d}, R^{1e} and R⁷ are defined herein below in Table 18.

TABLE 18

| Entry | R^{1a} | R^{1b} | R^{1c} | R^{1d} | R^{1e} | R⁷ |
|---|---|---|---|---|---|---|
| 1 | Cl | H | Cl | H | H | CH₃ |
| 2 | Cl | H | H | H | H | CH₃ |

Exemplary embodiments include compounds having the formula (XXVIII) or a pharmaceutically acceptable salt form thereof:

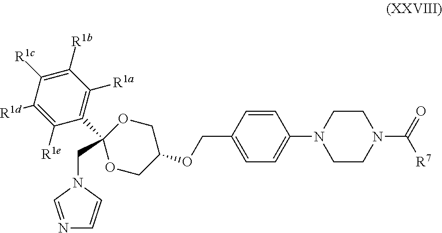

wherein non-limiting examples of R^{1a}, R^{1b}, R^{1c}, R^{1d}, R^{1e} and R⁷ are defined herein below in Table 19.

TABLE 19

| Entry | R^{1a} | R^{1b} | R^{1c} | R^{1d} | R^{1e} | R⁷ |
|---|---|---|---|---|---|---|
| 1 | Cl | H | Cl | H | H | CH₃ |
| 2 | Cl | H | H | H | H | CH₃ |

Exemplary embodiments include compounds having the formula (XXVIIIa) or a pharmaceutically acceptable salt form thereof:

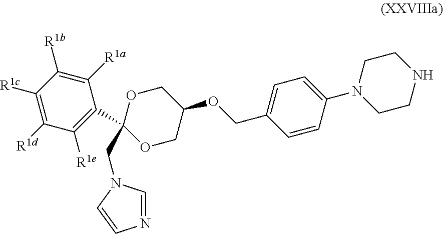

wherein non-limiting examples of R^{1a}, R^{1b}, R^{1c}, R^{1d}, R^{1e} and R⁷ are defined herein below in Table 20.

TABLE 20

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

Exemplary embodiments include compounds having the formula (XXVIIIb) or a pharmaceutically acceptable salt form thereof:

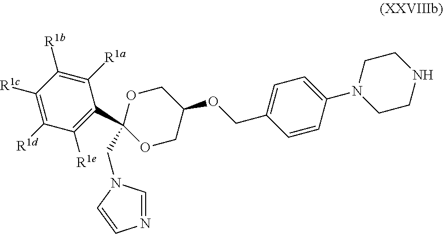

wherein non-limiting examples of R^{1a}, R^{1b}, R^{1c}, R^{1d}, R^{1e} and R⁷ are defined herein below in Table 21.

TABLE 21

| Entry | R^{1a} | R^{1b} | R^{1c} | R^{1d} | R^{1e} |
|---|---|---|---|---|---|
| 1 | Cl | H | Cl | H | H |
| 2 | Cl | H | H | H | H |

For the purposes of demonstrating the manner in which the compounds of embodiments described herein are named and referred to herein, the compound having the formula (XXIX)

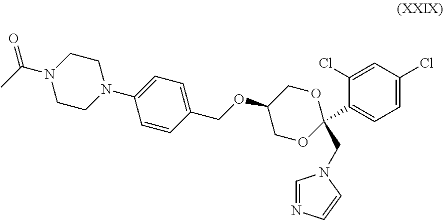 [SMILES: CC(=O)N1CCN(c2ccc(CO[C@H]3CO[C@](Cn4ccnc4)(c4ccc(Cl)cc4Cl)OC3)cc2)CC1]

has the chemical name 1-(4-(4-((((2s,5s)-2-((1H-imidazol-1-yl)methyl)-2-(2,4-dichlorophenyl)-1,3-dioxan-5-yl)oxy)methyl)phenyl)piperazin-1-yl)ethanone.

For the purposes of demonstrating the manner in which the compounds of embodiments described herein are named and referred to herein, the compound having the formula (XXX)

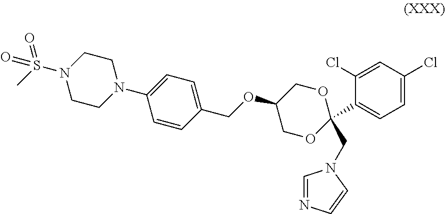 [SMILES: CS(=O)(=O)N1CCN(c2ccc(CO[C@H]3CO[C@](Cn4ccnc4)(c4ccc(Cl)cc4Cl)OC3)cc2)CC1]

has the chemical name 1-(4-((((2s,5 s)-2-((1H-imidazol-1-yl)methyl)-2-(2,4-dichlorophenyl)-1,3-dioxan-5-yl)oxy)methyl)phenyl)-4-(methylsulfonyl)piperazine.

For the purposes of demonstrating the manner in which the compounds of embodiments described herein are named and referred to herein, the compound having the formula (XXXI)

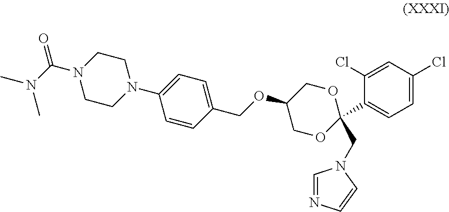 [SMILES: CN(C)C(=O)N1CCN(c2ccc(CO[C@H]3CO[C@](Cn4ccnc4)(c4ccc(Cl)cc4Cl)OC3)cc2)CC1]

has the chemical name 4-(4-((((2s,5s)-2-((1H-imidazol-1-yl)methyl)-2-(2,4-dichlorophenyl)-1,3-dioxan-5-yl)oxy)methyl)phenyl)-N,N-dimethylpiperazine-1-carboxamide.

For the purposes of demonstrating the manner in which the compounds of embodiments described herein are named and referred to herein, the compound having the formula (XXXII)

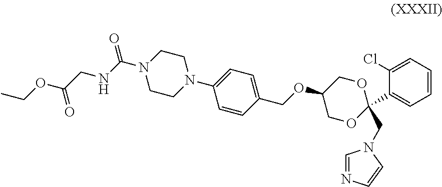 [SMILES: CCOC(=O)CNC(=O)N1CCN(c2ccc(CO[C@H]3CO[C@](Cn4ccnc4)(c4ccccc4Cl)OC3)cc2)CC1]

has the chemical name ethyl 2-(4-(4-((((2s,5s)-2-((1H-imidazol-1-yl)methyl)-2-(2-chlorophenyl)-1,3-dioxan-5-yl)oxy)methyl)phenyl)piperazine-1-carboxamido)acetate.

For the purposes of demonstrating the manner in which the compounds of embodiments described herein are named and referred to herein, the compound having the formula (XXXIII)

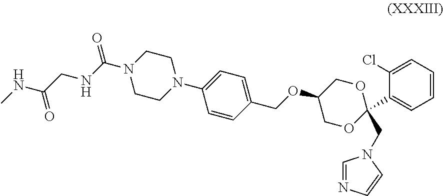 [SMILES: CNC(=O)CNC(=O)N1CCN(c2ccc(CO[C@H]3CO[C@](Cn4ccnc4)(c4ccccc4Cl)OC3)cc2)CC1]

has the chemical name 4-(4-((((2s,5s)-2-((1H-imidazol-1-yl)methyl)-2-(2-chlorophenyl)-1,3-dioxan-5-yl)oxy)methyl)phenyl)-N-(2-(methylamino)-2-oxoethyl)piperazine-1-carboxamide.

For the purposes of demonstrating the manner in which the compounds of embodiments described herein are named and referred to herein, the compound having the formula (XXXIV)

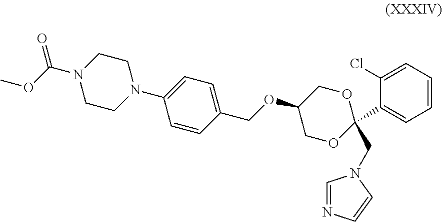 [SMILES: COC(=O)N1CCN(c2ccc(CO[C@H]3CO[C@](Cn4ccnc4)(c4ccccc4Cl)OC3)cc2)CC1]

has the chemical name methyl 4-(4-((((2s,5s)-2-((1H-imidazol-1-yl)methyl)-2-(2-chlorophenyl)-1,3-dioxan-5-yl)oxy)methyl)phenyl)piperazine-1-carboxylate.

For the purposes of demonstrating the manner in which the compounds of embodiments described herein are named and referred to herein, the compound having the formula (XXXV)

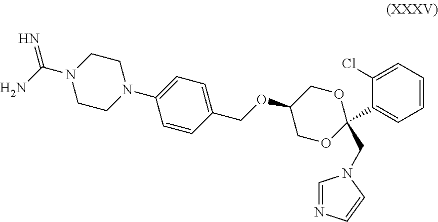 [SMILES: N=C(N)N1CCN(c2ccc(CO[C@H]3CO[C@](Cn4ccnc4)(c4ccccc4Cl)OC3)cc2)CC1]

has the chemical name 4-(4-((((2s,5s)-2-((1H-imidazol-1-yl)methyl)-2-(2-chlorophenyl)-1,3-dioxan-5-yl)oxy)methyl)phenyl)piperazine-1-carboximidamide.

For the purposes of demonstrating the manner in which the compounds of embodiments described herein are named and referred to herein, the compound having the formula (XXXVI)

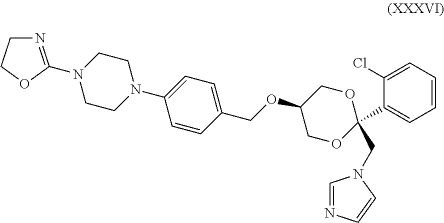 [SMILES: Clc1ccccc1[C@]1(Cn2ccnc2)OC[C@@H](OCc2ccc(N3CCN(C4=NCCO4)CC3)cc2)CO1]

has the chemical name 2-(4-(4-((((2s,5s)-2-((1H-imidazol-1-yl)methyl)-2-(2-chlorophenyl)-1,3-dioxan-5-yl)oxy)methyl)phenyl)piperazin-1-yl)-4,5-dihydrooxazole.

For the purposes of demonstrating the manner in which the compounds of embodiments described herein are named and referred to herein, the compound having the formula (XXXVII)

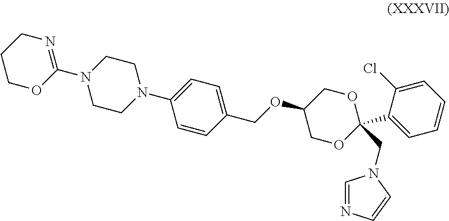 [SMILES: Clc1ccccc1[C@]1(Cn2ccnc2)OC[C@@H](OCc2ccc(N3CCN(C4=NCCCO4)CC3)cc2)CO1]

has the chemical name 2-(4-(4-((((2s,5s)-2-((1H-imidazol-1-yl)methyl)-2-(2-chlorophenyl)-1,3-dioxan-5-yl)oxy)methyl)phenyl)piperazin-1-yl)-5,6-dihydro-4H-1,3-oxazine.

In this document, a compound depicted by the racemic formula, for example:

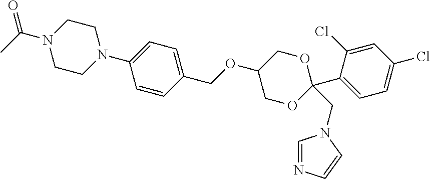 [SMILES: CC(=O)N1CCN(c2ccc(COC3COC(Cn4ccnc4)(c4ccc(Cl)cc4Cl)OC3)cc2)CC1]

will stand equally well for either of the two enantiomers having the formula:

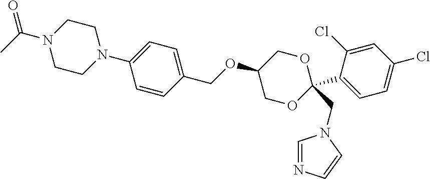 [SMILES: CC(=O)N1CCN(c2ccc(CO[C@H]3CO[C@](Cn4ccnc4)(c4ccc(Cl)cc4Cl)OC3)cc2)CC1]

or the formula:

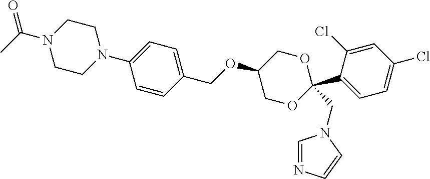 [SMILES: CC(=O)N1CCN(c2ccc(CO[C@H]3CO[C@@](Cn4ccnc4)(c4ccc(Cl)cc4Cl)OC3)cc2)CC1]

or mixtures thereof, or in the case where a third chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

### Process

Some embodiments of the present invention further relate to a process for preparing the cortisol lowering agents of embodiments described herein.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of embodiments described herein can be prepared according to the method illustrated in the General Synthetic Schemes.

### General Synthetic Schemes for Preparation of Compounds

The reagents used in the preparation of the compounds of this invention can be either commercially obtained or can be prepared by standard procedures described in the literature. In accordance with this invention, compounds in the genus may be produced by one of the following reaction schemes.

Compounds of the disclosure may be prepared according to the processes outlined in schemes 1-13.

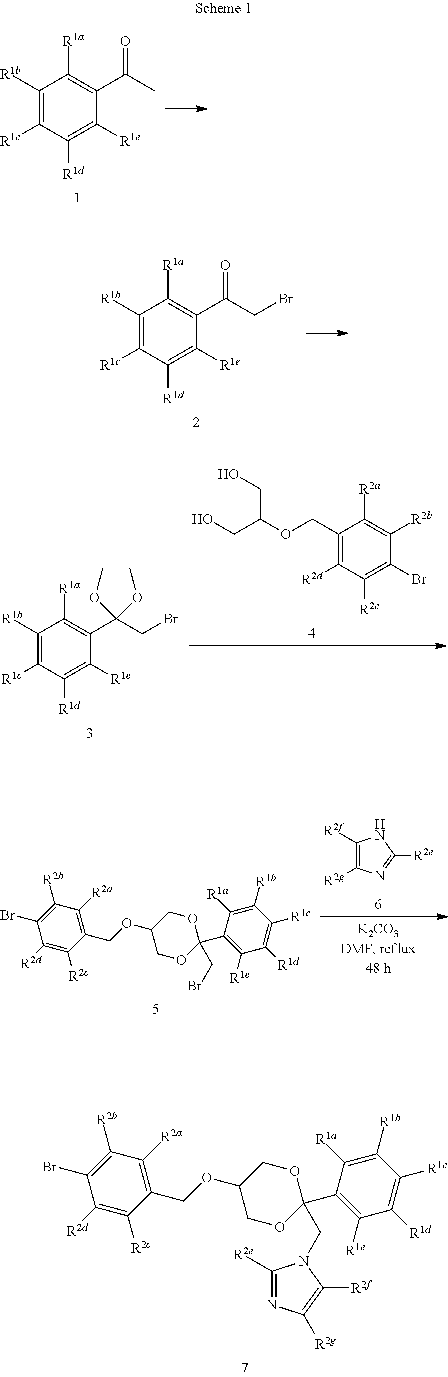

A suitably substituted compound of formula (1), a known compound or compound prepared by known methods, is reacted with a bromine in an organic solvent such as 1,4-dioxane, tetrahydrofuran, ethyl ether, methylene chloride, 1,2-dichloroethane, N,N-dimethylformamide, and the like to provide a compound of the formula (2). A compound of the formula (2) is then reacted with trimethyl orthoacetate in the presence of an acid such as p-toluenesulfonic acid, camphorsulfonic acid, hydrochloric acid, sulfuric acid, acetic acid, and the like in a solvent such as methanol, 1,4-dioxane, tetrahydrofuran, ethyl ether, methylene chloride, 1,2-dichloroethane, and the like optionally with heating to provide a compound of the formula (3). A compound of the formula (3) is then reacted with a compound of the formula (4), a known compound or compound prepared by known methods, in the presence of an acid such as p-toluenesulfonic acid, hydrochloric acid, sulfuric acid, acetic acid, and the like in a solvent such as benzene, toluene, p-xylene, 1,4-dioxane, tetrahydrofuran, and the like to provide a compound of the formula (5). A compound of the formula (5) is then reacted with a compound of the formula (6), a known compound or compound prepared by known methods, in the presence of a base such as potassium carbonate, sodium carbonate, lithium carbonate, cesium carbonate, sodium hydroxide, lithium hydroxide, potassium hydroxide, and the like, in a solvent such as N,N-dimethylformamide, N,N-dimethylacetamide, 1,4-dioxane, tetrahydrofuran, optionally with heating, optionally with microwave irradiation, to provide a compound of the formula (7).

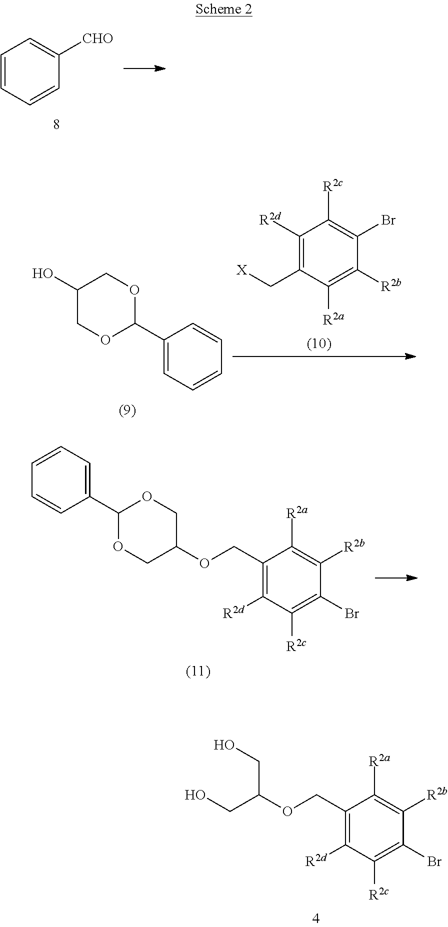

Benzaldehyde is reacted with a glycerol in an organic solvent such as 1,4-dioxane, tetrahydrofuran, 1,2-dichloroethane, N,N-dimethylformamide, N,N-dimethylacetamide, and the like, in the presence of an acid such as p-toluenesulfonic acid, camphorsulfonic acid, hydrochloric acid, sulfuric acid, acetic acid, and the like, optionally with heating, optionally with microwave irradiation, to provide a compound of the formula (9). A compound of the formula (9) is then reacted with a compound of the formula (10) wherein X is a leaving group such as bromine, chlorine, methansulfonate, and the like, in the presence of a base such as sodium hydride, potassium hydride, lithium diisopropylamide, sodium diisopropylamide, lithium bis(trimethylsilyl)amide, sodium bis(trimethylsilyl)amide, and the like in an solvent such as 1,4-dioxane, tetrahydrofuran, ethyl ether, methylene chloride, 1,2-dichloroethane, N,N-dimethylformamide, and the like to provide a compound of the formula (11). A compound of the formula (11) is then reacted with an acid such as p-toluenesulfonic acid, camphorsulfonic acid, hydrochloric acid, sulfuric acid, acetic acid, and the like, optionally with heating, optionally with microwave irradiation, to provide a compound of the formula (4).

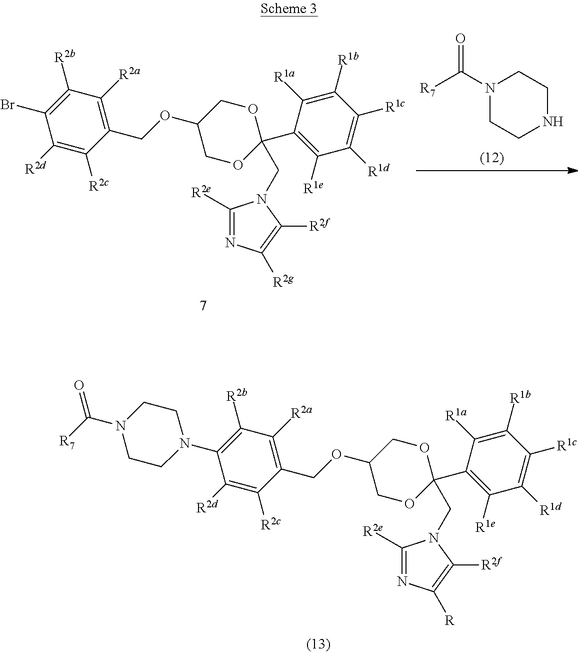

A compound of the formula (7) is reacted with a compound of the formula (12), a known compound or compound prepared by known methods, in the presence of a palladium catalyst such as palladium acetate, palladium bis(triphenylphosphine)dichloride, palladium tetrakis(triphenylphospine), bis(acetonitrile)dichloropalladium [1,1′-Bis(diphenylphosphino)ferrocene]dichloropalladium, and the like, optionally in the presence of 2-Dicyclohexylphosphino-2′,4′,6′-triisopropylbiphenyl (X-phos), in the presence of a base such as potassium carbonate, sodium carbonate, lithium carbonate, cesium carbonate, sodium hydroxide, lithium hydroxide, potassium hydroxide, and the like in a solvent such as toluene, benzene, p-xylene, 1,4-dioxane, tetrahydrofuran, and the like, optionally in the presence of water, optionally with heating, optionally with microwave irradiation, to provide a compound of the formula (13).

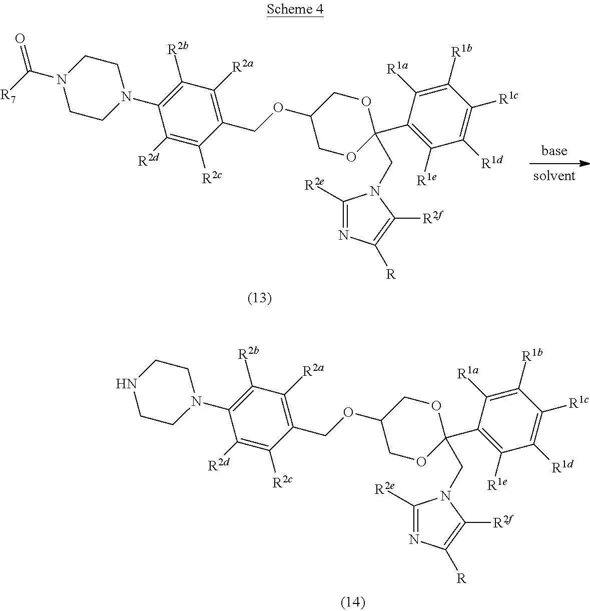

A compound of the formula (13) is reacted with a base such as a potassium carbonate, sodium carbonate, lithium carbonate, cesium carbonate, sodium hydroxide, lithium hydroxide, potassium hydroxide, and the like in a solvent such as methanol, ethanol, isopropanol, and the like, optionally with heating, optionally with microwave irradiation, to provide a compound of the formula (14).

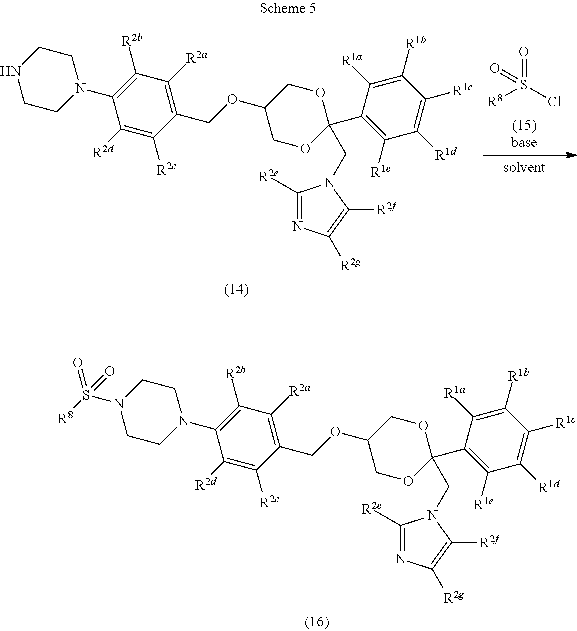

A suitably substituted compound of formula (14), a known compound or compound prepared by known methods, is reacted with a compound of the formula (15), a known compound or compound prepared by known methods, in the presence of a bases such as such as triethylamine, diisopropylethylamine, pyridine, 2,6-dimethylpyridine, N-methylmorpholine, and the like, in an organic solvent such as methylene chloride, dichloroethane, tetrahydrofuran, 1,4-dioxane, N,N-dimethylformamide, and the like to provide a compound of the formula (16).

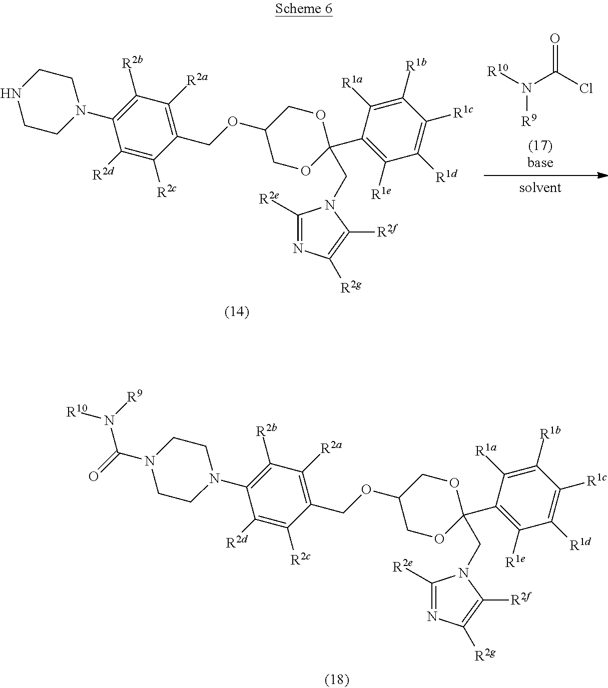

A suitably substituted compound of formula (14), a known compound or compound prepared by known methods, is reacted with a compound of the formula (17), a known compound or compound prepared by known methods, in the presence of a bases such as such as triethylamine, diisopropylethylamine, pyridine, 2,6-dimethylpyridine, N-methylmorpholine, and the like, in an organic solvent such as methylene chloride, dichloroethane, tetrahydrofuran, 1,4-dioxane, N,N-dimethylformamide, and the like to provide a compound of the formula (18).

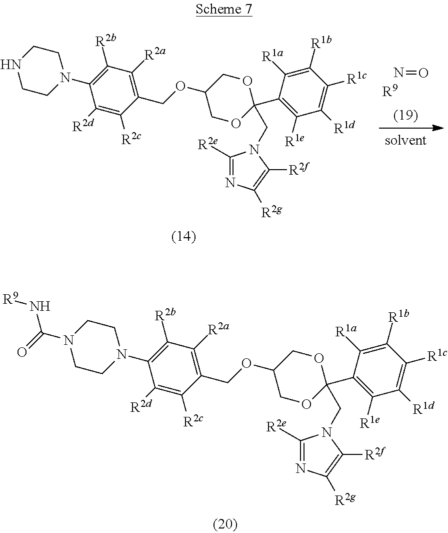

A suitably substituted compound of formula (14), a known compound or compound prepared by known methods, is reacted with a compound of the formula (19), a known compound or compound prepared by known methods in an organic solvent such as methylene chloride, dichloroethane, tetrahydrofuran, 1,4-dioxane, N,N-dimethylformamide, and the like to provide a compound of the formula (20).

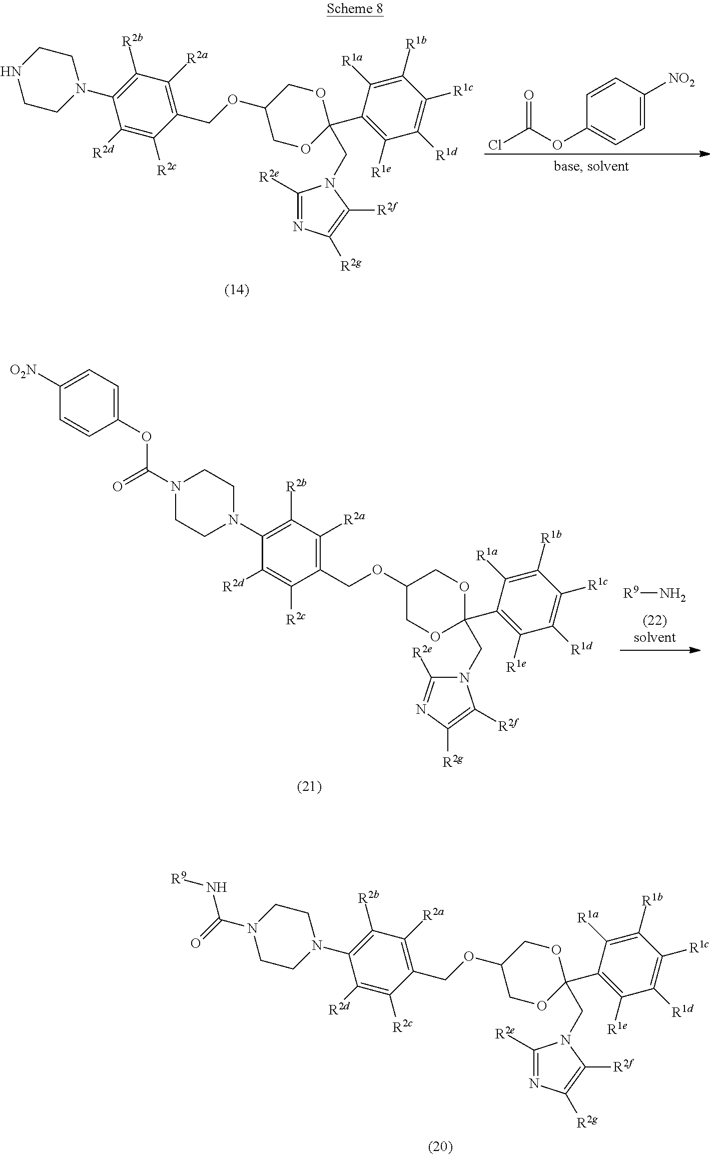

Alternatively, a suitably substituted compound of formula (14), a known compound or compound prepared by known methods, is reacted with a p-nitrophenylchloroformate in the presence of a bases such as such as triethylamine, diisopropylethylamine, pyridine, 2,6-dimethylpyridine, N-methylmorpholine, and the like, in an organic solvent such as methylene chloride, dichloroethane, tetrahydrofuran, 1,4-dioxane, N,N-dimethylformamide, and the like to provide a compound of the formula (21). A compound of formula (21) is then reacted with a compound of the formula (22), a known compound or compound prepared by known methods, in the presence of a bases such as such as triethylamine, diisopropylethylamine, pyridine, 2,6-dimethylpyridine, N-methylmorpholine, and the like, in an organic solvent such as methylene chloride, dichloroethane, tetrahydrofuran, 1,4-dioxane, N,N-dimethylformamide, and the like to provide a compound of the formula (20).

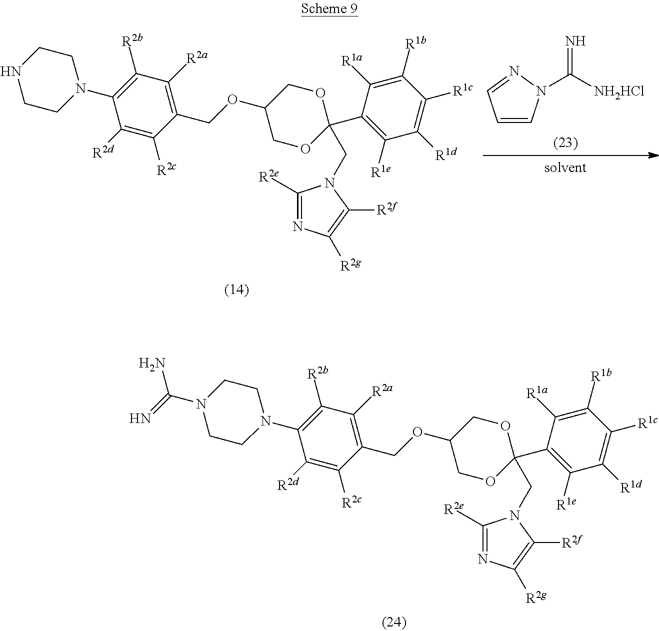

A suitably substituted compound of formula (14), a known compound or compound prepared by known methods, is reacted with a compound of the formula (23), in the presence of a bases such as such as triethylamine, diisopropylethylamine, pyridine, 2,6-dimethylpyridine, N-methylmorpholine, and the like, in an organic solvent such as methylene chloride, dichloroethane, tetrahydrofuran, 1,4-dioxane, N,N-dimethylformamide, and the like to provide a compound of the formula (24).

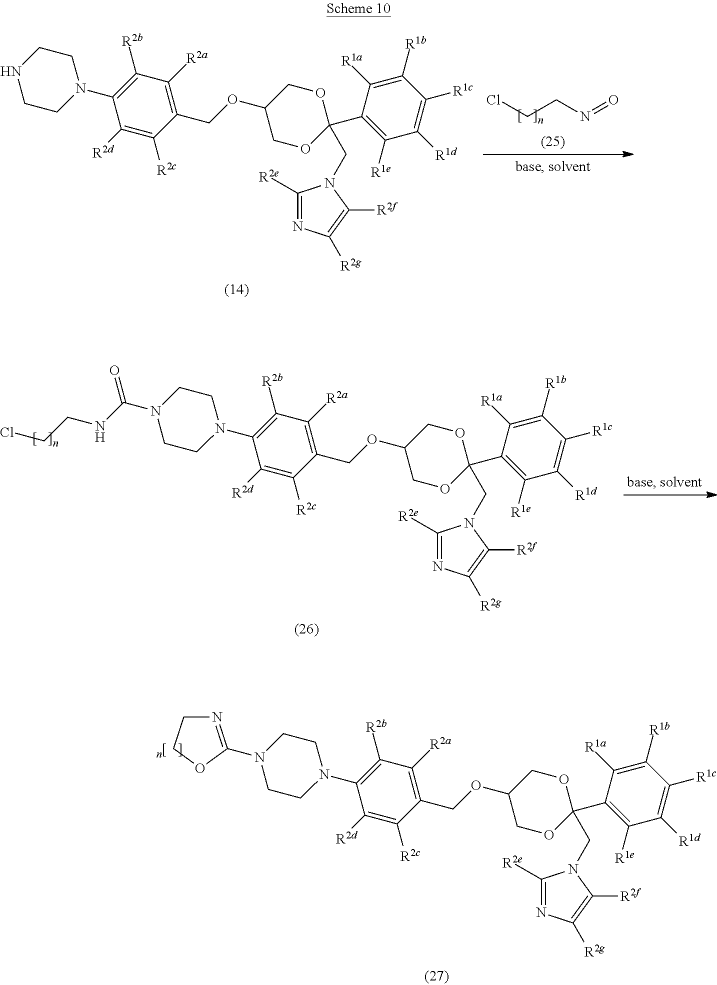

A suitably substituted compound of formula (14), a known compound or compound prepared by known methods, is reacted with a compound of the formula (25), a known compound or compound prepared by known methods wherein n is 1 or 2, in an organic solvent such as methylene chloride, dichloroethane, tetrahydrofuran, 1,4-dioxane, N,N-dimethylformamide, and the like to provide a compound of the formula (26). A compound of formula (26) is then reacted with a bases such as such as triethylamine, diisopropylethylamine, pyridine, 2,6-dimethylpyridine, N-methylmorpholine, potassium carbonate, sodium carbonate, lithium carbonate, and the like, in an organic solvent such as methylene chloride, dichloroethane, tetrahydrofuran, 1,4-dioxane, N,N-dimethylformamide, and the like to provide a compound of the formula (27).

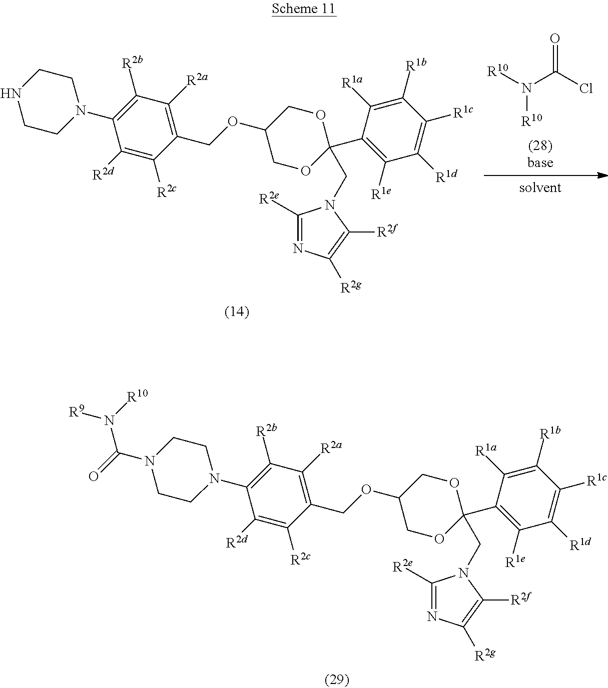

A suitably substituted compound of formula (14), a known compound or compound prepared by known methods, is reacted with a compound of the formula (28), a known compound or compound prepared by known methods, in the presence of a bases such as such as triethylamine, diisopropylethylamine, pyridine, 2,6-dimethylpyridine, N-methylmorpholine, and the like, in an organic solvent such as methylene chloride, dichloroethane, tetrahydrofuran, 1,4-dioxane, N,N-dimethylformamide, and the like to provide a compound of the formula (29).

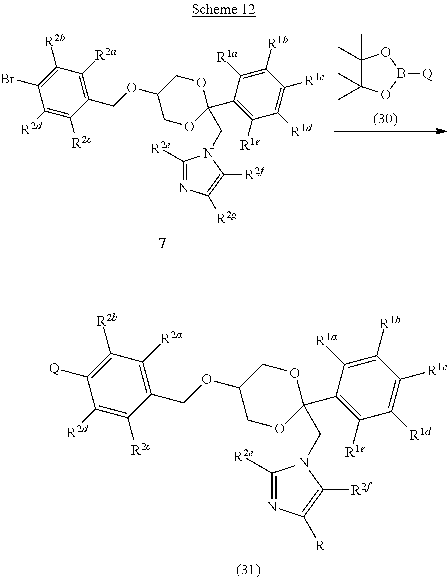

A compound of the formula (7) is reacted with a compound of the formula (30), a known compound or compound prepared by known methods, in the presence of a palladium catalyst such as palladium acetate, palladium bis(triphenylphosphine)dichloride, palladium tetrakis(triphenylphospine), bis(acetonitrile)dichloropalladium, [1,1′-Bis(diphenylphosphino)ferrocene]dichloropalladium and the like, optionally in the presence of 2-Dicyclohexylphosphino-2′,4′,6′-triisopropylbiphenyl (X-phos), in the presence of a base such as potassium carbonate, sodium carbonate, lithium carbonate, cesium carbonate, sodium hydroxide, lithium hydroxide, potassium hydroxide, and the like in a solvent such as toluene, benzene, p-xylene, 1,4-dioxane, tetrahydrofuran, and the like, optionally in the presence of water, optionally with heating, optionally with microwave irradiation, to provide a compound of the formula (31).

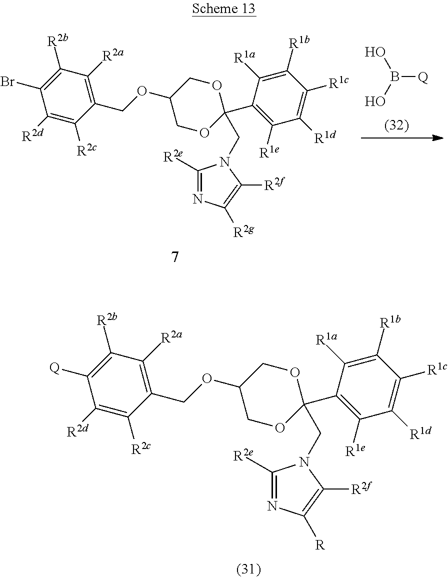

A compound of the formula (7) is reacted with a compound of the formula (32), a known compound or compound prepared by known methods, in the presence of a palladium catalyst such as palladium acetate, palladium bis(triphenylphosphine)dichloride, palladium tetrakis(triphenylphospine), bis(acetonitrile)dichloropalladium, [1,1′-Bis(diphenylphosphino)ferrocene]dichloropalladium and the like, optionally in the presence of 2-Dicyclohexylphosphino-2′,4′,6′-triisopropylbiphenyl (X-phos), in the presence of a base such as potassium carbonate, sodium carbonate, lithium carbonate, cesium carbonate, sodium hydroxide, lithium hydroxide, potassium hydroxide, and the like in a solvent such as toluene, benzene, p-xylene, 1,4-dioxane, tetrahydrofuran, and the like, optionally in the presence of water, optionally with heating, optionally with microwave irradiation, to provide a compound of the formula (31).

The Examples provided below provide representative methods for preparing exemplary compounds of embodiments described herein. The skilled practitioner will know how to substitute the appropriate reagents, starting materials and purification methods known to those skilled in the art, in order to prepare the compounds of embodiments described herein.

Examples 1-X provide methods for preparing representative compounds of the disclosure The skilled practitioner will know how to substitute the appropriate reagents, starting materials and purification methods known to those skilled in the art, in order to prepare additional compounds of embodiments described herein.

### EXAMPLE 1

### Synthesis of 2-Bromo-1-(2,4-dichlorophenyl)ethanone

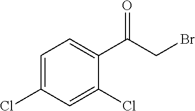 [SMILES: O=C(CBr)c1ccc(Cl)cc1Cl]

To a stirred solution of 1-(2,4-dichlorophenyl)ethanone (100 g, 0.528 mol) in 1,4-dioxane/ethyl ether (2:1, 600 mL) bromine (27 mL, 0.528 mol) was added drop wise at 0° C. over 1 h and the reaction mixture was then stirred for 1 hour. The reaction mixture was poured into ice-water and extracted with ethyl acetate. The organic layer was washed with water, brine and dried (Na₂SO₄) filtered and concentrated to give the title compound (145 g, 98%) as pale yellow liquid. The crude product was taken for the next step without any further purification. ¹H NMR (300 MHz, CDCl,) δ 7.56-7.52 (m, 1H), 7.48 (d, J=1.8 Hz, 1H), 7.35 (dd, J=1.8, 8.4 Hz, 1H), 4.49 (s, 2H).

The following compounds can be prepared by the procedure of 2-Bromo-1-(2,4-dichlorophenyl)ethanone. The skilled practitioner will know how to substitute the appropriate reagents, starting materials and purification methods known to those skilled in the art, in order to prepare the compounds provided herein.

### EXAMPLE 3

### Synthesis of 2-bromo-1-(2-chlorophenyl)ethanone

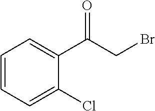 [SMILES: O=C(CBr)c1ccccc1Cl]

The title compound was prepared according to the procedure for 2-Bromo-1-(2,4-dichlorophenyl)ethanone, except 1-(2-chlorophenyl)ethanone was substituted for 1-(2,4-dichlorophenyl)ethanone. ¹H NMR (400 MHz, CDCl₃) δ 7.57 (d, J=7.6 Hz, 1H), 7.45 (d, J=3.6 Hz, 1H), 7.38-7.34 (m, 2H), 4.52 (s, 2H).

### EXAMPLE 4

### Synthesis of 1-(2-Bromo-1,1-dimethoxyethyl)-2,4-dichlorobenzene

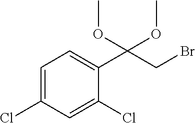

To a stirred solution of 2-Bromo-1-(2,4-dichlorophenyl)ethanone (145 g, 0.541 mol) in dry methanol (500 mL) was added trimethyl orthoformate (172 mL, 1.623 mol), followed by toluene-4-sulfonic acid monohydrate (10.29 g, 0.054 mol) at room temperature and the reaction mixture was refluxed for 12 hours. Reaction mixture was quenched with sodium methoxide at 0° C. and extracted with ethyl acetate. The organic layer was washed with water, brine, dried (Na₂SO₄) filtered and concentrated. The crude product was purified by column chromatography (100-200 mesh) eluting with 10% Ethyl acetate in petroleum ether to give the title compound. ¹H NMR: (400 MHz, CDCl₃) δ 7.79 (d, J=8.4 Hz, 1H), 7.40 (d, J=2.4 Hz, 1H), 7.29-7.26 (m, 1H), 3.89 (s, 2H), 3.22 (s, 6H).

The following compounds can be prepared by the procedure of 1-(2-Bromo-1,1-dimethoxyethyl)-2,4-dichlorobenzene. The skilled practitioner will know how to substitute the appropriate reagents, starting materials and purification methods known to those skilled in the art, in order to prepare the compounds provided herein.

### EXAMPLE 5

### Synthesis of 1-(2-bromo-1,1-dimethoxyethyl)-2-chlorobenzene

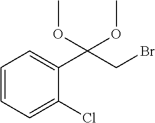

The title compound was prepared according to the procedure for 1-(2-Bromo-1,1-dimethoxyethyl)-2,4-dichlorobenzene, except 2-bromo-1-(2-chlorophenyl)ethanone was substituted for 2-Bromo-1-(2,4-dichlorophenyl)ethanone. ¹H NMR: (300 MHz, CDCl₃) δ 7.87-7.82 (m, 1H), 7.38-7.35 (m, 1H), 7.29-7.25 (m, 2H), 3.93 (s, 2H), 3.22 (s, 6H).

### EXAMPLE 6

### Synthesis of (2s,5s)-5-((4-bromobenzyl)oxy)-2-(bromomethyl)-2-(2,4-dichlorophenyl)-1,3-dioxane and (2r,5r)-5-((4-bromobenzyl)oxy)-2-(bromomethyl)-2-(2,4-dichlorophenyl)-1,3-dioxane

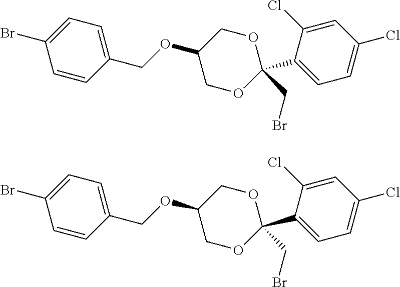

A mixture of 1-(2-Bromo-1,1-dimethoxyethyl)-2,4-dichlorobenzene (15 g, 0.0477 mol) and 2-(4-Bromobenzyloxy)propane-1,3-diol (12.4 g, 0.0477 mol) in dry benzene (150 mL) was refluxed for 1 hour. After 1 hour solvent was distilled out (50 mL) and cooled to 60° C. Toluene-4-sulfonic acid monohydrate was added at 60° C. to reaction mixture and refluxed for 3 hours. The reaction mixture was quenched with triethylamine, diluted with water and extracted with ethyl acetate. The organic layer was washed with water, brine, dried (Na₂SO₄) filtered and concentrated to give title compound as a mixture of isomers. The isomers were separated by column chromatography (100-200 mesh) using 10% ethyl acetate in petroleum ether. (2s,5s)-5-((4-bromobenzyl)oxy)-2-(bromomethyl)-2-(2,4-dichlorophenyl)-1,3-dioxane: ¹H NMR: (400 MHz, CDCl₃) δ 7.56 (d, J=8.8 Hz, 1H), 7.48-7.46 (m, 3H), 7.35 (dd, J=2.0, 8.4 Hz, 1H), 7.16 (d, J=8.4 Hz, 2H), 4.48 (s, 2H), 4.15 (q, 2H), 3.9-3.83 (m, 1H), 3.61 (s, 2H), 3.44 (t, J=10.4 Hz, 2H). (2r,5r)-5-((4-bromobenzyl)oxy)-2-(bromomethyl)-2-(2,4-dichlorophenyl)-1,3-dioxane: ¹H NMR: (400 MHz, CDCl₃) δ 7.55 (d, J=8.8 Hz, 1H), 7.48-7.45 (m, 3H), 7.32-7.26 (m, 3H), 4.6 (s, 2H), 4.12 (d, J=12.0 Hz, 2H), 3.78 (d, J=12.4 Hz, 2H), 3.68 (s, 2H), 3.26 (t, J=2.0 Hz, 1H).

The following compounds can be prepared by the procedure of (2s,5s)-5-((4-bromobenzyl)oxy)-2-(bromomethyl)-2-(2,4-dichlorophenyl)-1,3-dioxane and (2r,5r)-5-((4-bromobenzyl)oxy)-2-(bromomethyl)-2-(2,4-dichlorophenyl)-1,3-dioxane. The skilled practitioner will know how to substitute the appropriate reagents, starting materials and purification methods known to those skilled in the art, in order to prepare the compounds provided herein.

### EXAMPLE 7

### Synthesis of (2s,5s)-5-((4-bromobenzyl)oxy)-2-(bromomethyl)-2-(2-chlorophenyl)-1,3-dioxane and (2r,5r)-5-((4-bromobenzyl)oxy)-2-(bromomethyl)-2-(2-chlorophenyl)-1,3-dioxane

The title compounds were prepared according to the procedure for (2s,5s)-5-((4-bromobenzyl)oxy)-2-(bromomethyl)-2-(2,4-dichlorophenyl)-1,3-dioxane and (2r,5r)-5-((4-bromobenzyl)oxy)-2-(bromomethyl)-2-(2,4-dichlorophenyl)-1,3-dioxane was substituted for 1-(2-bromo-1,1-dimethoxyethyl)-2,4-dichlorobenzene.

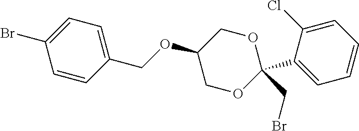 [SMILES: Clc1ccccc1[C@]1(CBr)OC[C@@H](OCc2ccc(Br)cc2)CO1]

(2s,5s)-5-((4-bromobenzyl)oxy)-2-(bromomethyl)-2-(2-chlorophenyl)-1,3-dioxane. ¹H NMR: (300 MHz, CDCl₃) δ 7.60-7.55 (m, 1H), 7.45-7.39 (m, 3H), 7.37-7.27 (m, 2H), 7.13-7.10 (m, 2H), 4.46 (s, 2H), 4.14-4.09 (m, 2H), 3.94-3.82 (m, 1H), 3.62 (s, 2H), 3.49-3.42 (m, 2H).

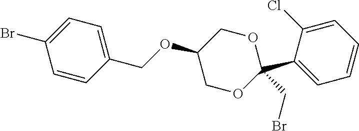

(2r,5r)-5-((4-bromobenzyl)oxy)-2-(bromomethyl)-2-(2-chlorophenyl)-1,3-dioxane. ¹H NMR: (300 MHz, CDCl₃) δ 7.64-7.59 (m, 1H), 7.48-7.40 (m, 3H), 7.33-7.26 (m, 4H), 4.62 (s, 2H), 4.14-4.10 (m, 2H), 3.83-3.79 (m, 2H), 3.70 (s, 2H), 3.25-3.23 (m, 1H).

### EXAMPLE 8

### Synthesis of 1-(((2s,5s)-5-((4-bromobenzyl)oxy)-2-(2,4-dichlorophenyl)-1,3-dioxan-2-yl)methyl)-1H-imidazole

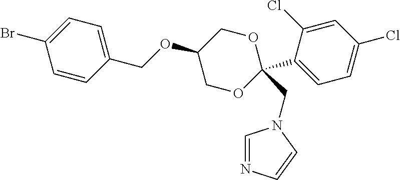 [SMILES: Clc1ccc([C@]2(Cn3ccnc3)OC[C@@H](OCc3ccc(Br)cc3)CO2)c(Cl)c1]

To a stirred solution of (2s,5s)-5-((4-bromobenzyl)oxy)-2-(bromomethyl)-2-(2,4-dichlorophenyl)-1,3-dioxane (11g, 0.0215 mol) in dry N,N-dimethylformamide (120 mL) potassium carbonate (14.87 g, 0.107 mol) was added, followed by imidazole (14.65 g, 0.215 mol) at room temperature and the reaction mixture was refluxed for 48 hours. The reaction mixture was filtered to remove the precipitated solid and the filtrate was poured into ice-water and extracted with ethyl acetate. The organic layer was washed with water, brine and dried (Na₂SO₄) filtered and concentrated and crude material was purified by column chromatography on silica (100-200 mesh) eluting with 50% ethyl acetate in petroleum ether to give the title compound. ¹H NMR: (400 MHz, CDCl₃) δ 7.45 (m, 3H), 7.27 (d, J=5.6 Hz, 1H), 7.17 (s, 2H), 7.11 (d, J=8.4 Hz, 2H), 6.9 (s, 1H), 6.71 (s, 1H), 4.42 (s, 2H), 4.17 (s, 2H), 4.03 (q, 2H), 3.72-3.64 (m, 1H), 3.36 (t, J=9.6 Hz, 2H); LCMS: m/z=498.19 [(M+H)⁺]; (Purity: 98%); IR (thin film): 2875.67, 1585.52, 1467.39, 1107.06, 804.91 cm⁻¹.

### EXAMPLE 9

### Synthesis of 1-(((2r,5r)-5-((4-bromobenzyl)oxy)-2-(2,4-dichlorophenyl)-1,3-dioxan-2-yl)methyl)-1H-imidazole (7b)

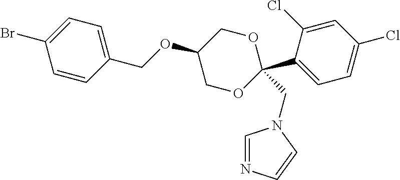 [SMILES: Clc1ccc([C@]2(Cn3ccnc3)OC[C@H](OCc3ccc(Br)cc3)CO2)c(Cl)c1]

To a stirred solution of (2r,5r)-5-((4-bromobenzyl)oxy)-2-(bromomethyl)-2-(2,4-dichlorophenyl)-1,3-dioxane (15 g, 0.0293 mol) in dry N,N-dimethylformamide (120 mL) potassium carbonate (20.2 g, 0.146 mol) was added, followed by imidazole (19.98 g, 0.293 mol) at room temperature and the reaction mixture was refluxed for 48 hours. The reaction mixture was filtered and the filtrate was poured into ice-water and extracted with ethyl acetate. The organic layer was washed with water, brine and dried (Na₂SO₄) filtered and concentrated and the crude material was purified by column chromatography on silica (100-200 mesh) eluting with 50% ethyl acetate in petroleum ether to give the title compound. ¹H NMR: (400 MHz, CDCl₃) δ 7.5-7.45 (m, 3H), 7.24 (d, J=4 Hz, 2H), 7.21 (s, 1H), 7.15 (dd, J=2, 8, 8.0 Hz, 1H), 7.09 (d, J=8.0 Hz, 1H), 6.87 (s, 1H), 6.7 (s, 1H), 4.56 (s, 2H), 4.29 (s, 2H), 4.07 (d, J=11.6 Hz, 2H), 3.74 (d, J=11.6 2H), 3.21 (s, 1H); LCMS: m/z=498. 19 [(M+H)^{|}].

The following compounds can be prepared by the procedure of 1-(((2s,5s)-5-((4-bromobenzyl)oxy)-2-(2,4-dichlorophenyl)-1,3-dioxan-2-yl)methyl)-1H-imidazole. The skilled practitioner will know how to substitute the appropriate reagents, starting materials and purification methods known to those skilled in the art, in order to prepare the compounds provided herein.

### EXAMPLE 10

### Synthesis of 1-(((2s,5s)-5-((4-bromobenzyl)oxy)-2-(2-chlorophenyl)-1,3-dioxan-2-yl)methyl)-1H-imidazole

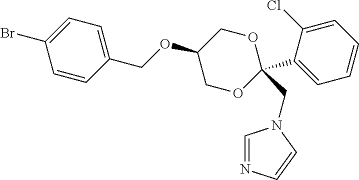 [SMILES: Clc1ccccc1[C@]1(Cn2ccnc2)OC[C@@H](OCc2ccc(Br)cc2)CO1]

The title compounds were prepared according to the procedure for 1-(((2s,5s)-5-((4-bromobenzyl)oxy)-2-(2,4-dichlorophenyl)-1,3-dioxan-2-yl)methyl)-1H-imidazole, except (2s,5s)-5-((4-bromobenzyl)oxy)-2-(bromomethyl)-2-(2-chlorophenyl)-1,3-dioxane was substituted for (2s,5s)-5-((4-bromobenzyl)oxy)-2-(bromomethyl)-2-(2,4-dichlorophenyl)-1,3-dioxane. ¹H NMR: (400 MHz, CDCl₃) 7.45-7.42 (m, 3H), 7.30-7.27 (m, 2H), 7.25-7.24 (m, 1H), 7.22-7.17 (m, 1H), 7.11 (d, J=8.4 Hz, 2H), 6.89 (s, 1H), 6.72 (s, 1H), 4.42 (s, 2H), 4.19 (s, 2H), 4.04-3.99 (m, 2H), 3.72-3.65 (m, 1H), 3.41-3.37 (m, 2H); ESIMS: m/z=462.9 [(M+H)⁺].

### EXAMPLE 11

### Synthesis of 1-(((2r,5r)-5-((4-bromobenzyl)oxy)-2-(2-chlorophenyl)-1,3-dioxan-2-yl)methyl)-1H-imidazole

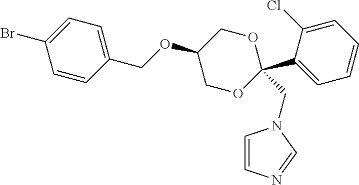 [SMILES: Clc1ccccc1[C@]1(Cn2ccnc2)OC[C@H](OCc2ccc(Br)cc2)CO1]

The title compounds were prepared according to the procedure for 1-(((2s,5s)-5-((4-bromobenzyl)oxy)-2-(2,4-dichlorophenyl)-1,3-dioxan-2-yl)methyl)-1H-imidazole, except (2r,5r)-5-((4-bromobenzyl)oxy)-2-(bromomethyl)-2-(2-chlorophenyl)-1,3-dioxane was substituted for (2s,5s)-5-((4-bromobenzyl)oxy)-2-(bromomethyl)-2-(2,4-dichlorophenyl)-1,3-dioxane. ¹H NMR: (400 MHz, CDCl₃) δ 7.49 (d, J=8.4 Hz, 2H), 7.44 (d, J=8.0 Hz, 1H), 7.29-7.27 (m, 1H), 7.23-7.21 (m, 3H), 7.17-7.15 (m, 2H), 6.85 (s, 1H), 6.70 (s, 1H), 4.56 (s, 2H), 4.30 (s, 2H), 4.09-4.05 (m, 2H), 3.80-3.75 (m, 2H), 3.21-3.20 (m, 1H); ESIMS: m/z=462.9 [(M+H)⁺].

### EXAMPLE 12

### Synthesis of 5-(4-Bromobenzyloxy)-2-phenyl-1,3-dioxane

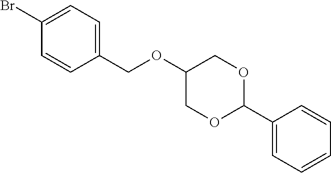 [SMILES: Brc1ccc(COC2COC(c3ccccc3)OC2)cc1]

To a stirred solution of benzaldehyde (200 g, 1.88 mol) in dry N,N-dimethylformamide (500 mL) was added glycerol (344 mL, 4.71 mol), followed by camphor-10-sulphonic acid (43.8 g, 0.188 mol) at room temperature and the reaction mixture was heated to 70° C. for 2 hours. The reaction mixture was poured into ice-water and extracted with ethyl acetate and washed with 10% sodium bicarbonate solution. The organic layer was washed with water, brine and dried (Na₂SO₄) filtered and concentrated. The crude product was purified by column chromatography on silica (100-200 mesh) eluting with 15% ethyl acetate in petroleum ether to give an oil. The oil was dissolved in tetrahydrofuran (200 mL) and added dropwise over 45 minutes to a suspension of NaH (53.33 g, 2.22 mol) in tetrahydrofuran (800 mL). After 45 minutes 4-bromobenzylbromide (277.7 g, 1.11 mol) was added portionwise over 30 minutes and the reaction mixture was stirred at room temperature for 6 hours. The reaction mixture was poured into ice-water and extracted with ethyl acetate. The organic layer was washed with water, brine, dried (Na₂SO₄) filtered and concentrated. The crude product was washed with petroleum ether and the precipitated solid was filtered. The solid product was purified by column chromatography on silica (100-200 mesh) eluting with 10% ethyl acetate in petroleumether to give the title compound. ¹H NMR: (300 MHz, CDCl₃) δ 7.49-7.44 (m, 4H), 7.37-7.34 (m, 3H), 7.21 (d, J=8.4 Hz, 2H), 5.39 (s, 1H), 4.53 (s, 2H), 4.37 (q, 2H), 3.77-3.72 (m, 1H), 3.63 (t, J=9.3 Hz, 2H); LCMS: m/z=349.2 [(M+H)⁺].

### EXAMPLE 13

### Synthesis of 2-(4-Bromobenzyloxy)propane-1,3-diol

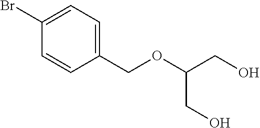 [SMILES: OCC(CO)OCc1ccc(Br)cc1]

To a stirred solution of 5-(4-Bromobenzyloxy)-2-phenyl-1,3-dioxane (20 g, 0.057 mol) in methanol (150 mL) dilute HCl (0.5 N, 65 mL) was added dropwie at 0° C. over 15 minutes and the reaction mixture was refluxed for 30 minutes. The reaction mixture was poured into ice-water and washed with petroleum ether (2×150 mL). The aqueous layer was basified with 10% sodium bicarbonate solution and extracted with ethyl acetate and washed with water, brine, dried (Na₂SO₄) filtered and concentrated to give the title compound. ¹H NMR: (300 MHz, DMSO-d₆) δ 7.53 (d, J=8.4 Hz, 2H), 7.34 (d, J=7.8 Hz, 2H), 4.58 (s, 2H), 4.56 (bs, 2H), 3.52-3.32 (m, 5H); IR (thin film): 3271.13, 1485.44, 1341.71, 1120.78, 1070.56, 802.64 cm⁻¹.

### EXAMPLE 14

### Synthesis of 1-(4-(4-((((2s,5s)-2-((1H-imidazol-1-yl)methyl)-2-(2,4-dichlorophenyl)-1,3-dioxan-5-yl)oxy)methyl)phenyl)piperazin-1-yl)ethanone

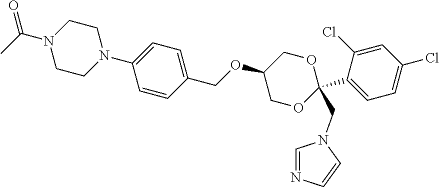 [SMILES: CC(=O)N1CCN(c2ccc(CO[C@H]3CO[C@](Cn4ccnc4)(c4ccc(Cl)cc4Cl)OC3)cc2)CC1]

To a stirred solution of 1-(((2s,5s)-5-((4-bromobenzyl)oxy)-2-(2,4-dichlorophenyl)-1,3-dioxan-2-yl)methyl)-1H-imidazole (4.5 g, 9.03 mmol) in dry toluene (70 mL) was added 1-acetylpiperazine (1.5 g, 11.74 mmol) followed by Cs₂CO₃ (5.88 g, 18.06 mmol) and the reaction mixture was degassed with argon for 10 minutes. X-Phos (0.43 g, 0.903 mmol), palladium acetate (0.20 g, 0.903 mmol) were then added and again degassed with argon for 10 minutes. The reaction mixture was refluxed over 2 hours. The reaction mixture was filtered and concentrated. The crude product was purified by column chromatography on silica (100-200 mesh) eluting with 10% methanol in ethyl acetate to give the title compound. ¹H-NMR: (400 MHz, CDCl₃) δ 7.44 (s, 1H), 7.29 (s, 1H), 7.16 (s, 1H), 7.15 (d, J=8.0 Hz, 3H), 6.91 (s, 1H), 6.87 (d, J=8.8 Hz, 2H), 6.71 (s, 1H), 4.39 (s, 2H), 4.17 (s, 2H), 4.0 (q, 2H), 3.76 (t, J=5.6 Hz, 2H), 3.69-3.65 (m, 1H), 3.61 (t, J=5.2 Hz, 2H), 3.36 (t, J=9.2 Hz, 2H), 3.18-3.12 (m, 4H), 2.13 (s, 3H); LCMS: m/z=545.45 [(M+H)⁺]. IR (thin film): 3465.45, 2923.08, 1643.94, 1436.44, 1233.85, 1078.20, 803.77 cm⁻¹.

### EXAMPLE 15

### Synthesis of 1-(4-(4-((((2r,5r)-2-((1H-imidazol-1-yl)methyl)-2-(2,4-dichlorophenyl)-1,3-dioxan-5-yl)oxy)methyl)phenyl)piperazin-1-yl)ethanone

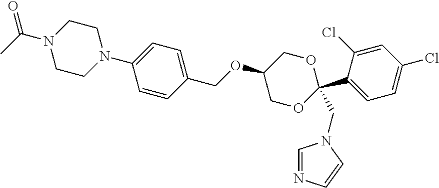 [SMILES: CC(=O)N1CCN(c2ccc(CO[C@H]3CO[C@@](Cn4ccnc4)(c4ccc(Cl)cc4Cl)OC3)cc2)CC1]

To a stirred solution of 1-(((2r,5r)-5-((4-bromobenzyl)oxy)-2-(2,4-dichlorophenyl)-1,3-dioxan-2-yl)methyl)-1H-imidazole (0.5 g, 1.00 mmol) in dry toluene (20 mL) was added 1-acetylpiperazine (0.16 g, 1.304 mmol) followed by Cs₂CO₃ (0.65 g, 2.00 mmol) and the reaction mixture was degassed with argon for 10 minutes. X-Phos (0.047 g, 0.100 mmol), palladium acetate (0.022 g, 0.100 mmol) were added and again degassed with argon for 10 min. The reaction mixture was then refluxed for 12 hours. The reaction mixture was filtered and concentrated. The residue was purified by column chromatography on silica (100-200 mesh) eluting with 10% methanol in ethyl acetate to give the title compound. (400 MHz, CDCl₃) δ 7.45 (d, J=2.0 Hz, 1H), 7.34 (s, 1H), 7.23 (d, J=10.0 Hz, 2H), 7.12 (d, J=2.0 Hz, 1H), 7.09 (d, J=8.0 Hz, 1H), 6.92 (d, J=8.8 Hz, 2H), 6.88 (s, 1H), 6.71 (s, 1H), 4.53 (s, 2H), 4.3 (s, 2H), 4.06 (d, J=11.6 Hz, 2H), 3.78 (t, J=5.6 Hz, 2H), 3.7 (t, J=7.2 Hz, 2H), 3.63 (t, J=5.6 Hz, 2H), 3.2 (s, 1H), 3.17 (m, 4H), 2.14 (s, 3H); LCMS: m/z=545.47 [(M+H)⁺].

### EXAMPLE 16

### Synthesis of 1-(4-((((2s,5s)-2-((1H-imidazol-1-yl)methyl)-2-(2,4-dichlorophenyl)-1,3-dioxan-5-yl)oxy)methyl)phenyl)piperazine

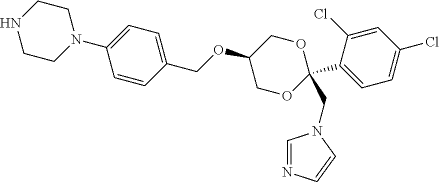 [SMILES: Clc1ccc([C@]2(Cn3ccnc3)OC[C@@H](OCc3ccc(N4CCNCC4)cc3)CO2)c(Cl)c1]

To a stirred solution of 1-(4-(4-((((2s,5s)-2-((1H-imidazol-1-yl)methyl)-2-(2,4-dichlorophenyl)-1,3-dioxan-5-yl)oxy)methyl)phenyl)piperazin-1-yl)ethanone (0.6 g, 1.1 mmol) in methanol (5 mL) was added 30% NaOH (1 mL) and the reaction mixture was refluxed for 16 hours. The reaction mixture was poured into water and extracted with methylene chloride. The organic layer was washed with water, brine, dried (Na₂SO₄) filtered and concentrated to give the title compound. ¹H-NMR: (400 MHz, DMSOd6) δ 7.66 (d, J=1.6 Hz, 1H), 7.41-7.38 (dd, J=1.6, 8 Hz, 1H), 7.25 (m, 1H), 7.09 (d, J=9.2 Hz, 2H), 6.85 (d, J=8.8 Hz, 2H), 6.8 (s, 1H), 6.74 (s, 1H), 4.35 (s, 2H), 4.26 (s, 2H), 4.05 (q, 2H), 3.63 (m, 1H), 3.19 (t, J=20.4 Hz, 2H), 3 (t, J=10 Hz, 4H), 2.8 (t, J=9.2 Hz, 4H), 2.28 (bs, 1H). LCMS: m/z=502.14 [(M+H)^{|}].

### EXAMPLE 17

### Synthesis of 3-((4-(4-((((2s,5s)-2-((1H-imidazol-1-yl)methyl)-2-(2,4-dichlorophenyl)-1,3-dioxan-5-yl)oxy)methyl)phenyl)piperazin-1-yl)sulfonyl)benzonitrile

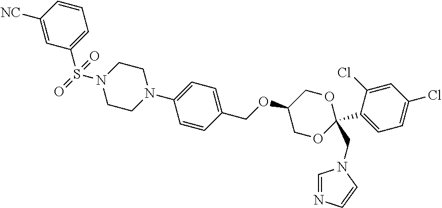 [SMILES: N#Cc1cccc(S(=O)(=O)N2CCN(c3ccc(CO[C@H]4CO[C@](Cn5ccnc5)(c5ccc(Cl)cc5Cl)OC4)cc3)CC2)c1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.05 g, 0.099 mmol) in dry methylene chloride (5 mL) was added triethylamine (0.069 mL, 0.497 mmol) followed by 3-cyanobenzene sulfonyl chloride (0.024 g, 0.119 mmol) and the reaction mixture was stirred at room temperature over 1 hour. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% Methanol in methylene chloride) to give the title compound. ¹H-NMR: (300 MHz, CDCl₃) δ 8.07 (s, 1H), 8.02 (d, J=8.1 Hz, 1H), 7.91 (d, J=7.5 Hz, 1H), 7.73-7.68 (m, 1H), 7.44 (s, 1H), 7.29 (s, 1H), 7.16-7.11 (m, 4H), 6.9 (s, 1H), 6.28 (d, J=8.7 Hz, 2H), 6.71 (s, 1H), 4.37 (s, 2H), 4.16 (s, 2H), 4.0 (q, 2H), 3.67-3.62 (m, 1H), 3.36 (d, J=9.9 Hz, 2H), 3.23-3.21 (m, 8H). LCMS: m/z=670 [(M+H)⁺].

### EXAMPLE 18

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-((3-chloropropyl)sulfonyl)piperazine

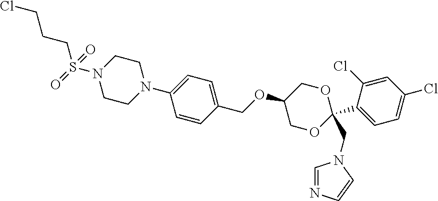 [SMILES: O=S(=O)(CCCCl)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.08 g, 0.159 mmol) in dry methylene chloride (5 mL) was added triethylamine (0.11 mL, 0.795 mmol) followed by 3-chloropropane sulfonyl chloride (0.023 mL, 0.190 mmol) and the reaction mixture was stirred at room temperature over 1 hour. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% Methanol in methylene chloride) to give the title compound. ¹H-NMR: (300 MHz, CDCl₃) δ 7.46 (s, 2H), 7.2 (s, 2H), 7.16 (d, J=8.4 Hz, 2H), 6.97 (s, 1H), 6.88 (d, J=8.7 Hz, 2H), 6.76 (s, 1H), 4.39 (s, 2H), 4.18 (s, 2H), 4.0 (q, 2H), 3.74-3.68 (m, 3H), 3.44 (t, J=9.9 Hz, 4H), 3.38(d, J=9.9 Hz, 2H), 3.25 (t, J=9.6 Hz, 4H), 3.12 (t, J=14.7 Hz, 2H), 2.35-2.30 (m, 2H). LCMS: m/z=643 [(M+H)⁺].

### EXAMPLE 19

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-(cyclopropylsulfonyl)piperazine

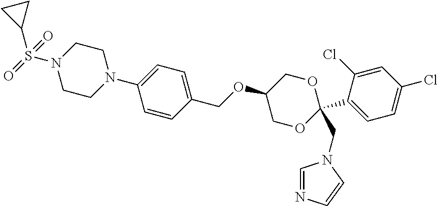 [SMILES: O=S(=O)(C1CC1)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.08 g, 0.159 mmol) in dry methylene chloride (5 mL) was added triethylamine (0.11 mL, 0.795 mmol) followed by cyclopropane sulfonyl chloride (0.026 g, 0.190 mmol) and the reaction mixture was stirred at room temperature over 2 hours. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% methanol in methylene chloride) to give the title compound. ¹H-NMR: (400 MHz, CDCl₃) δ 7.45 (s, 1H), 7.30 (s, 2H), 7.17-7.14 (m, 3H), 6.91 (s, 1H), 6.88 (d, J=8.4 Hz, 2H), 6.71 (s, 1H), 4.39 (s, 2H), 4.17 (s, 2H), 4.01 (q, 2H), 3.69-3.65 (m, 1H), 3.45 (t, J=10 Hz, 4H), 3.25 (t, J=10 Hz, 4H). LCMS: m/z=606.14 [(M+H)⁺].

### EXAMPLE 20

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-(isopropylsulfonyl)piperazine

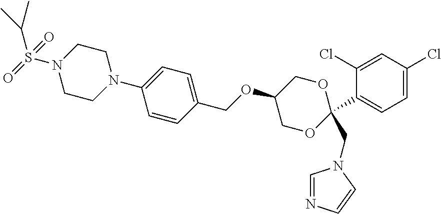 [SMILES: CC(C)S(=O)(=O)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.09 g, 0.178 mmol) in dry methylene chloride (5 mL) was added triethylamine (0.12 mL, 0.894 mmol) followed by 2-propane sulfonyl chloride (0.03 g, 0.214 mmol) and the reaction mixture was stirred at room temperature over 1 hour. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% methanol in methylene chloride) to give the title compound. ¹H-NMR: (300 MHz, CDCl₃) δ 7.44 (s, 1H), 7.17-7.13 (m, 5H), 6.91 (s, 1H), 6.88 (d, J=8.4 Hz, 2H), 6.73 (s, 1H), 4.39 (s, 2H), 4.17 (s, 2H), 4.0 (q, 2H), 3.67-3.66 (m, 1H), 3.50 (t, J=9.6 Hz, 4H), 3.37 (d, J=10.2 Hz, 2H), 3.20-3.19 (m, 5H), 1.38 (s, 3H); 1.36 (s, 3H). LCMS: m/z=608.16 [(M+H)⁺].

### EXAMPLE 21

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-(ethylsulfonyl)piperazine

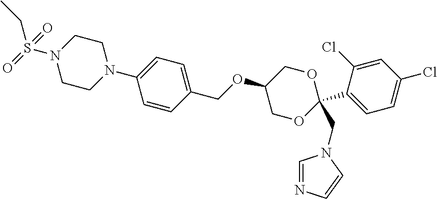 [SMILES: CCS(=O)(=O)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.09 g, 0.178 mmol) in dry methylene chloride (5 mL) was added triethylamine (0.12 mL, 0.894 mmol) followed by ethane sulfonyl chloride (0.027 g, 0.214 mmol) and the reaction mixture was stirred at room temperature over 1 hour. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% Methanol in methylene chloride) to give the title compound. ¹H-NMR: (400 MHz, CDCl₃) δ 7.45 (s, 1H), 7.32 (s, 1H), 7.17-7.14 (m, 4H), 6.91 (s, 1H), 6.88 (d, J=8.4 Hz, 2H), 6.72 (s, 1H), 4.39 (s, 2H), 4.17 (s, 2H), 4.01 (q, 2H), 3.67 (m, 1H), 3.44 (t, J=10 Hz, 4H), 3.34 (s, 2H), 3.23 (t, J=10 Hz, 4H), 3.02 (q, 2H), 1.40 (t, J=15.2 Hz, 3H). LCMS: m/z=594.14 [(M+H)⁺].

### EXAMPLE 22

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-((1H-imidazol-4-yl)sulfonyl)piperazine

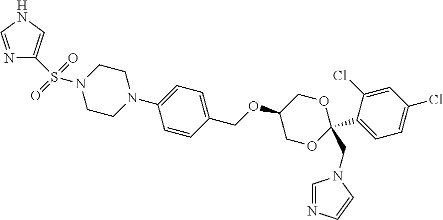 [SMILES: O=S(=O)(c1c[nH]cn1)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.09 g, 0.178 mmol) in dry methylene chloride (5 mL) was added triethylamine (0.12 mL, 0.894 mmol) followed by 1H-imidazole-4-sulfonyl chloride (0.035 g, 0.214 mmol) and the reaction mixture was stirred at room temperature over 1 hour. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% methanol in methylene chloride) to give the title compound. ¹H-NMR: (300 MHz, CDCl₃) δ 7.71 (s, 1H), 7.62(s, 1H), 7.45 (s, 2H), 7.19 (s, 2H), 7.12 (d, J=8.4 Hz, 2H), 6.93 (s, 1H), 6.82 (d, J=8.7 Hz, 2H), 6.72 (s, 1H), 4.37 (s, 2H), 4.16 (s, 2H), 3.99 (q, 2H), 3.68-3.65 (m, 1H), 3.32 (s, 1H), (m, 8H), 3.23-3.22 (m, 8H). LCMS: m/z=632.13 [(M+H)⁺].

### EXAMPLE 23

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-((3-(trifluoromethoxy)phenyl)sulfonyl)piperazine

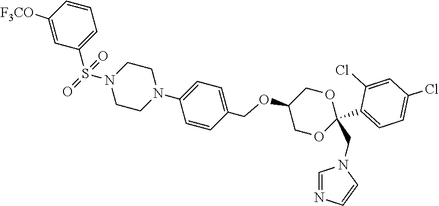 [SMILES: O=S(=O)(c1cccc(OC(F)(F)F)c1)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.09 g, 0.178 mmol) in dry methylene chloride (5 mL) was added triethylamine (0.12 mL, 0.894 mmol) followed by 3-(trifluoromethoxy)benzene sulfonyl chloride (0.055 g, 0.214 mmol) and the reaction mixture was stirred at room temperature over 2 hours. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% Methanol in methylene chloride) to give the title compound. ¹H-NMR: (400 MHz, CDCl₃) δ 7.73 (d, J=8 Hz, 1H), 7.64-7.59 (m, 2H), 7.48 (d, J=7.2 Hz 1H), 7.44 (s, 1H), 7.16 (s, 2H),7.13 (d, J=8 Hz, 2H), 6.9 (s, 1H), 6.82 (d, J=8 Hz 2H), 6.71 (s, 1H), 4.37 (s, 2H), 4.16 (s, 2H), 3.99 (q, 2H), 3.67-3.63 (m, 1H), 3.32 (s, 2H), 3.23-3.18 (m, 8H). LCMS: m/z=726.12 [(M+H)⁺].

### EXAMPLE 24

### Synthesis of 1-(4-((((2s,5s)-2-((1H-imidazol-1-yl)methyl)-2-(2,4-dichlorophenyl)-1,3-dioxan-5-yl)oxy)methyl)phenyl)-4-(pyridin-3-ylsulfonyl)piperazine

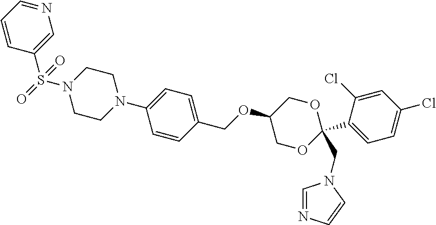 [SMILES: O=S(=O)(c1cccnc1)N1CCN(c2ccc(CO[C@H]3CO[C@](Cn4ccnc4)(c4ccc(Cl)cc4Cl)OC3)cc2)CC1]

To a stirred solution of 1-(4-((((2s,5s)-2-((1H-imidazol-1-yl)methyl)-2-(2,4-dichlorophenyl)-1,3-dioxan-5-yl)oxy)methyl)phenyl)piperazine (0.08 g, 0.159 mmol) in dry methylene chloride (5 mL) was added triethylamine (0.11 mL, 0.795 mmol) followed by pyridine-3-sulfonyl chloride (0.022 mL, 0.190 mmol) and the reaction mixture was stirred at room temperature over 1 hour. The reaction mixture was poured into ice-water and extracted with methylene chloride and washed with 10% sodium bicarbonate solution. The organic layer was washed with water, brine, dried (Na₂SO₄) filtered and concentrated. The crude product was purified by prep TLC (10% methanol in methylene chloride) to give the title compound. ¹H-NMR: (400 MHz, CDCl₃) δ9.02 (d, J=2 Hz, 1H), 8.85-8.84 (m, 1H), 8.08 (d, J=8 Hz, 1H), 7.52-7.49 (m, 1H), 7.44 (s, 1H), 7.26 (s, 1H), 7.15-7.11 (m, 3H), 6.89 (s, 1H), 6.82 (d, J=8.4 Hz, 2H), 6.71 (s, 1H), 4.37 (s, 2H), 4.16 (s, 2H), 3.99 (q, 2H), 3.68-3.63 (m, 1H), 3.32 (s, 2H), 3.23-3.22 (m, 8H). LCMS: m/z=643.14 [(M+H)⁺].

### EXAMPLE 26

### Synthesis of 1-(4-(4-((((2s,5s)-2-((1H-imidazol-1-yl)methyl)-2-(2-chlorophenyl)-1,3-dioxan-5-yl)oxy)methyl)phenyl)piperazin-1-yl)ethanone

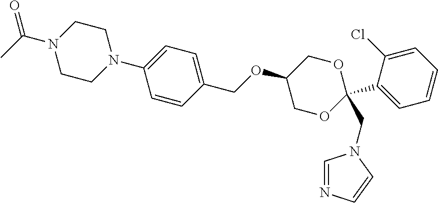 [SMILES: CC(=O)N1CCN(c2ccc(CO[C@H]3CO[C@](Cn4ccnc4)(c4ccccc4Cl)OC3)cc2)CC1]

To a stirred solution of 1-(((2s,5s)-5-((4-bromobenzyl)oxy)-2-(2-chlorophenyl)-1,3-dioxan-2-yl)methyl)-1H-imidazole (0.4 g, 0.87 mmol) in dry toluene (18 mL) was added 1-acetylpiperazine (0.13 g, 1.04 mmol) followed by Cs₂CO₃ (0.56 g, 1.74 mmol) and the reaction mixture was degassed with argon for 10 minutes, X-Phos (0.041 g, 0.09 mmol), palladium acetate (0.019 g, 0.09 mmol) were then added and again degassed with argon for 10 minutes. The reaction mixture was refluxed over 2 hours. The reaction mixture was filtered and concentrated and the residue was purified by column chromatography on silica (100-200 mesh) eluting with 10% methanol in ethyl acetate to afford the title compound. ¹H-NMR: (400 MHz, CDCl₃) δ 7.43 (d, J=7.6 Hz, 1H), 7.30-7.26 (m, 2H), 7.25-7.23 (m, 1H), 7.20 (d, J=6.8 Hz, 1H), 7.16 (d, J=8.4 Hz, 2H), 6.89-6.85 (m, 3H), 6.71 (s, 1H), 4.39 (s, 2H), 4.19 (s, 2H), 4.01-3.97 (m, 2H), 3.77-3.74 (m, 2H), 3.72-3.64 (m, 1H), 3.62-3.59 (m, 2H), 3.45-3.35 (m, 2H), 3.18-3.12 (m, 4H), 2.13 (s, 3H); ESIMS: m/z=510.9 [(M+H)⁺].

### EXAMPLE 27

### Synthesis of 1-(4-(4-((((2r,5r)-2-((1H-imidazol-1-yl)methyl)-2-(2-chlorophenyl)-1,3-dioxan-5-yl)oxy)methyl)phenyl)piperazin-1-yl)ethanone

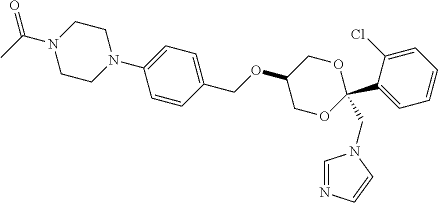

To a stirred solution of 1-(((2r,5r)-5-((4-bromobenzyl)oxy)-2-(2-chlorophenyl)-1,3-dioxan-2-yl)methyl)-1H-imidazole (0.4 g, 0.87 mmol) in dry toluene (18 mL) was added 1-acetylpiperazine (0.13 g, 1.04 mmol) followed by Cs₂CO₃ (0.56 g, 1.74 mmol) and the reaction mixture was degassed with argon for 10 minutes. X-Phos (0.041 g, 0.09 mmol), palladium acetate (0.019 g, 0.09 mmol) were then added and again degassed with argon for 10 minutes. The reaction mixture was refluxed over 2 hours. The reaction mixture was filtered and concentrated. The crude product was purified by column chromatography on silica (100-200 mesh) eluting with 10% methanol in ethyl acetate to afford the title compound. ¹H NMR: (300 MHz, CDCl₃) δ 7.67-7.65 (m, 1H), 7.60-7.55 (m, 2H), 7.42-7.38 (m, 2H), 7.30-7.28 (m, 1H), 7.26-7.22 (m, 3H), 6.92-6.89 (m, 2H), 4.48 (s, 2H), 4.41 (s, 2H), 4.07-4.02 (m, 2H), 3.94-3.90 (m, 1H), 3.80-3.75 (m, 4H), 3.64-3.62 (m, 2H), 3.20-3.14 (m, 4H), 2.14 (s, 3H).; ESIMS: m/z=510.9 [(M+H)⁺].

### EXAMPLE 28

### Synthesis of 4-(4-((((2s,5s)-2-((1H-imidazol-1-yl)methyl)-2-(2,4-dichlorophenyl)-1,3-dioxan-5-yl)oxy)methyl)phenyl)pyridine

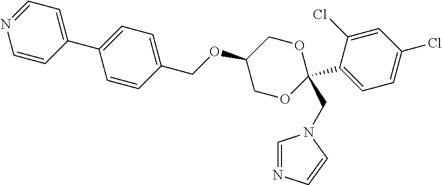 [SMILES: Clc1ccc([C@]2(Cn3ccnc3)OC[C@@H](OCc3ccc(-c4ccncc4)cc3)CO2)c(Cl)c1]

To a stirred solution of 1-(((2s,5s)-5-((4-bromobenzyl)oxy)-2-(2-chlorophenyl)-1,3-dioxan-2-yl)methyl)-1H-imidazole (0.3 g, 0.602 mmol) in 1,4-Dioxane/water (9:1, 10 mL) was added K₂CO₃ (0.24 g, 1.80 mmol), pyridine-4-boronicacid pinacol ester (0.148 g, 0.722 mmol) and the reaction mixture was degassed with nitrogen gas for 10 minutes. [1,1′-Bis(diphenylphosphino)ferrocene]dichloropalladium (0.044 g, 0.0602 mmol), was added and again degassed with nitrogen gas for 10 min. The reaction mixture was then refluxed for 12 hours. The reaction mixture was filtered and concentrated. The crude product was purified by prep TLC (10% Methanol in methylene chloride) to give the title compound. ¹H-NMR: (400 MHz, CDCl₃) δ8.65 (d, J=6 Hz, 2H), 7.6 (d, J=8.1 Hz, 2H), 7.48-7.46 (m, 3H), 7.36 (d, J=8.1 Hz, 2H), 7.29 (s, 1H), 7.29 (s, 1H), 7.18 (d, J=0.9 Hz, 2H), 6.9 (s, 1H), 6.73 (s, 1H), 4.53 (s, 2H), 4.18 (s, 2H), 4.07 (q, 2H), 3.76-3.7 (m, 1H), 3.39 (t, J=20.1 Hz, 2H). LCMS: m/z=495.11 [(M+H)⁺].

### EXAMPLE 29

### Synthesis of 4-(4-((((2s,5s)-2-((1H-imidazol-1-yl)methyl)-2-(2-chlorophenyl)-1,3-dioxan-5-yl)oxy)methyl)phenyl)morpholine

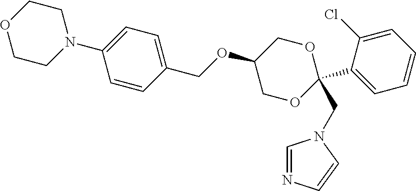 [SMILES: Clc1ccccc1[C@]1(Cn2ccnc2)OC[C@@H](OCc2ccc(N3CCOCC3)cc2)CO1]

To a stirred solution of 1-(((2s,5s)-5-((4-bromobenzyl)oxy)-2-(2-chlorophenyl)-1,3-dioxan-2-yl)methyl)-1H-imidazole (0.28 g, 0.61 mmol) in dry toluene (5 mL) was added morpholine (0.063 g, 0.73 mmol) followed by Cs₂CO₃ (0.39 g, 1.22 mmol) and the reaction mixture was degassed with argon for 10 minutes. X-Phos (0.028 g, 0.06 mmol), palladium acetate (0.013 g, 0.06 mmol) were then added and again degassed with argon for 10 minutes. The reaction mixture was refluxed over 2 hours. The reaction mixture was filtered, concentrated and the residue was purified by prep TLC to afford the title compound. ¹H NMR: (300 MHz, CDCl₃) δ 7.43-7.40 (m, 1H), 7.26-7.13 (m, 6H), 6.90-6.83 (m, 3H), 6.71 (s, 1H), 4.39 (s, 2H), 4.19 (s, 2H), 4.01-3.96 (m, 2H), 3.86-3.83 (m, 4H), 3.72-3.63 (m, 1H), 3.42-3.31 (m, 2H), 3.15-3.12 (m, 4H); ESIMS: m/z=469.9 [(M+H)⁺].

### EXAMPLE 30

### Synthesis of 4-(4-((((2r,5r)-2-((1H-imidazol-1-yl)methyl)-2-(2-chlorophenyl)-1,3-dioxan-5-yl)oxy)methyl)phenyl)morpholine

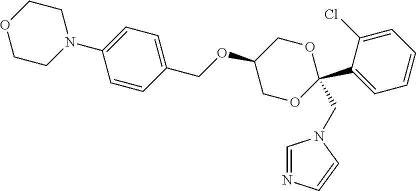 [SMILES: Clc1ccccc1[C@]1(Cn2ccnc2)OC[C@H](OCc2ccc(N3CCOCC3)cc2)CO1]

To a stirred solution of (2r,5r)-5-((4-bromobenzyl)oxy)-2-(bromomethyl)-2-(2-chlorophenyl)-1,3-dioxane (0.2 g, 0.43 mmol) in dry toluene (5 mL) was added morpholine (0.045 g, 0.5 mmol) followed by Cs₂CO₃ (0.28 g, 0.86 mmol) and the reaction mixture was degassed with argon for 10 minutes. X-Phos (0.02 g, 0.04 mmol), palladium acetate (0.010 g, 0.04 mmol) were then added and again degassed with argon for 10 minutes. The reaction mixture was refluxed over 2 hours. The reaction mixture was filtered, concentrated and the residue was purified by prep TLC to afford the title compound. ¹H NMR: (400 MHz, CDCl₃) δ7.43 (d, J=8.0 Hz, 1H), 7.26-7.24 (m, 1H), 7.23-7.19 (m, 3H), 7.12-7.11 (m, 2H), 6.90 (d, J=8.0 Hz, 2H), 6.83 (s, 1H), 6.66 (s, 1H), 4.56 (s, 2H), 4.32 (s, 2H), 4.06-4.03 (m, 2H), 3.87-3.84 (m, 4H), 3.76-3.73 (m, 2H), 3.18-3.14 (m, 5H); ESIMS: m/z=469.9 [(M+H)⁺].

The following compounds can be prepared by the procedure of 4-(4-((((2s,5s)-2-((1H-imidazol-1-yl)methyl)-2-(2-chlorophenyl)-1,3-dioxan-5-yl)oxy)methyl)phenyl)morpholine. The skilled practitioner will know how to substitute the appropriate reagents, starting materials and purification methods known to those skilled in the art, in order to prepare the compounds provided herein.

### EXAMPLE 31

### Synthesis of 1-(4-((((2s,5s)-2-((1H-imidazol-1-yl)methyl)-2-(2-chlorophenyl)-1,3-dioxan-5-yl)oxy)methyl)phenyl)-4-(methylsulfonyl)piperazine

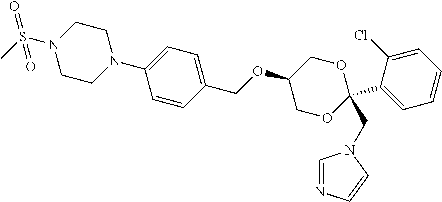 [SMILES: CS(=O)(=O)N1CCN(c2ccc(CO[C@H]3CO[C@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except 1-(methylsulfonyl)piperazine was substituted for morpholine. ¹H NMR: (400 MHz, CDCl₃) δ 7.43 (d, J=7.2 Hz, 1H), 7.29-7.26 (m, 2H), 7.25-7.24 (m, 1H), 7.20-7.14 (m, 3H), 6.90-6.86 (m, 6H), 6.72 (s, 1H), 4.39 (s, 2H), 4.19 (s, 2H), 4.02-3.97 (m, 2H), 3.71-3.64 (m, 1H), 3.38-3.36 (m, 6H), 3.28-3.25 (m, 4H), 2.82 (s, 3H); ESIMS: m/z=547.03 [(M+H)⁺].

### EXAMPLE 32

### Synthesis of 1-(4-((((2r,5r)-2-((1H-imidazol-1-yl)methyl)-2-(2-chlorophenyl)-1,3-dioxan-5-yl)oxy)methyl)phenyl)-4-(methylsulfonyl)piperazine

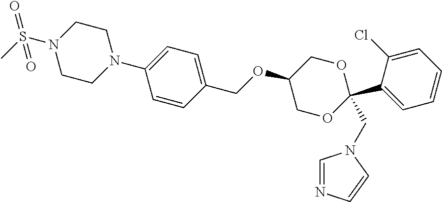 [SMILES: CS(=O)(=O)N1CCN(c2ccc(CO[C@H]3CO[C@@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except 1-(methylsulfonyl)piperazine was substituted for morpholine and (2r,5r)-5-((4-bromobenzyl)oxy)-2-(bromomethyl)-2-(2-chlorophenyl)-1,3-dioxane was substituted for (2s,5s)-5-((4-bromobenzyl)oxy)-2-(bromomethyl)-2-(2-chlorophenyl)-1,3-dioxane. ¹H NMR: (400 MHz, CDCl₃) δ 7.43 (d, J=7.2 Hz, 1H), 7.29-7.26 (m, 2H), 7.25-7.24 (m, 1H), 7.20-7.14 (m, 3H), 6.90-6.86 (m, 6H), 6.72 (s, 1H), 4.39 (s, 2H), 4.19 (s, 2H), 4.02-3.97 (m, 2H), 3.71-3.64 (m, 1H), 3.38-3.36 (m, 6H), 3.28-3.25 (m, 4H), 2.82 (s, 3H); ESIMS: m/z=547.03 [(M+H)⁺].

### Example 33

### Synthesis of 2-methoxyethyl 4-(4-((((2s,5s)-2-((1H-imidazol-1-yl)methyl)-2-(2-chlorophenyl)-1,3-dioxan-5-yl)oxy)methyl)phenyl)piperazine-1-carboxylate

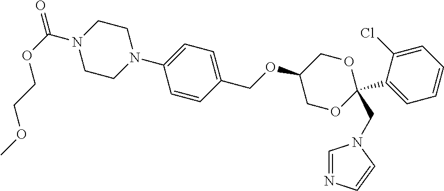 [SMILES: COCCOC(=O)N1CCN(c2ccc(CO[C@H]3CO[C@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except 2-methoxyethyl piperazine-1-carboxylate was substituted for morpholine. ¹H NMR: (400 MHz, CDCl₃) δ 7.43-7.41 (m, 1H), 7.31-7.27 (m, 2H), 7.26-7.18 (m, 2H), 7.16-7.13 (m, 2H), 6.91 (s, 1H), 6.87 (d, J=8.8 Hz, 2H), 6.71 (s, 1H), 4.38 (s, 2H), 4.28-4.25 (m, 2H), 4.19 (s, 2H), 4.01-3.97 (m, 2H), 3.71-3.60 (m, 7H), 3.50-3.39 (m, 5H), 3.14-3.11 (m, 4H).; ESIMS: m/z=571.1 [(M+H)⁺].

### EXAMPLE 34

### Synthesis of 2-methoxyethyl 4-(4-((((2r,5r)-2-((1H-imidazol-1-yl)methyl)-2-(2-chlorophenyl)-1,3-dioxan-5-yl)oxy)methyl)phenyl)piperazine-1-carboxylate

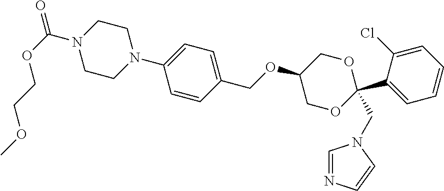 [SMILES: COCCOC(=O)N1CCN(c2ccc(CO[C@H]3CO[C@@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except 2-methoxyethyl piperazine-1-carboxylate was substituted for morpholine and (2r,5r)-5-((4-bromobenzyl)oxy)-2-(bromomethyl)-2-(2-chlorophenyl)-1,3-dioxane was substituted for (2s,5s)-5-((4-bromobenzyl)oxy)-2-(bromomethyl)-2-(2-chlorophenyl)-1,3-dioxane. ¹H NMR: (400 MHz, CDCl₃) δ 7.43 (d, J=8.0 Hz, 1H), 7.24-7.20 (m, 3H), 7.14-7.11 (m, 3H), 6.91 (d, J=8.4 Hz, 2H), 6.83 (s, 1H), 6.66 (s, 1H), 4.55 (s, 2H), 4.32 (s, 2H), 4.28-4.26 (m, 2H), 4.06-4.03 (m, 2H), 3.76-3.73 (m, 2H), 3.65-3.61 (m, 6H), 3.39 (s, 3H), 3.18-3.14 (m, 5H); ESIMS: m/z=571.3 [(M+H)⁺].

### EXAMPLE 35

### Synthesis of ethyl 2-(4-(4-((((2s,5s)-2-((1H-imidazol-1-yl)methyl)-2-(2-chlorophenyl)-1,3-dioxan-5-yl)oxy)methyl)phenyl)piperazine-1-carboxamido)acetate

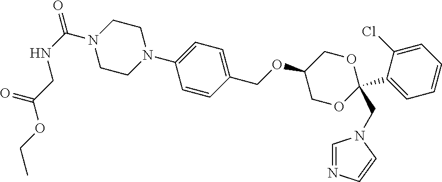 [SMILES: CCOC(=O)CNC(=O)N1CCN(c2ccc(CO[C@H]3CO[C@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except ethyl 2-(piperazine-1-carboxamido)acetate was substituted for morpholine. ¹H NMR: (400 MHz, CDCl₃) δ 7.43 (d, J=7.2 Hz, 1H), 7.28-7.26 (m, 1H), 7.25-7.23 (m, 2H), 7.20 (d, J=8.0 Hz, 1H), 7.15 (d, J=8.4 Hz, 2H), 6.86-6.84 (m, 3H), 6.75-6.65 (m, 1H), 5.02-4.98 (m, 1H), 4.39 (s, 2H), 4.25-4.19 (m, 4H), 4.03-3.97 (m, 4H), 3.69-3.65 (m, 1H), 3.56 (t, J=5.2 Hz, 4H), 3.42-3.35 (m, 2H), 3.18 (t, J=5.6 Hz, 4H), 1.31 (q, J=6.8 Hz, 3H); ESIMS: m/z=598.1 [(M+H)⁺].

### EXAMPLE 36

### Synthesis of ethyl 2-(4-(4-((((2r,5r)-2-((1H-imidazol-1-yl)methyl)-2-(2-chlorophenyl)-1,3-dioxan-5-yl)oxy)methyl)phenyl)piperazine-1-carboxamido)acetate

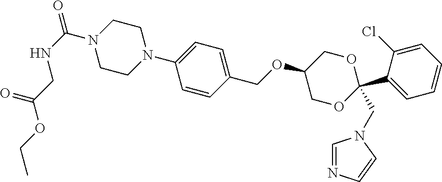 [SMILES: CCOC(=O)CNC(=O)N1CCN(c2ccc(CO[C@H]3CO[C@@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except ethyl 2-(piperazine-1-carboxamido)acetate was substituted for morpholine and (2r,5r)-5-((4-bromobenzyl)oxy)-2-(bromomethyl)-2-(2-chlorophenyl)-1,3-dioxane was substituted for (2s,5s)-5-((4-bromobenzyl)oxy)-2-(bromomethyl)-2-(2-chlorophenyl)-1,3-dioxane. ¹H NMR: (400 MHz, CDCl₃) δ 7.43-7.41 (m, 1H), 7.31-7.22 (m, 4H), 7.12-7.11 (m, 2H), 6.91-6.83 (m, 3H), 6.70-6.67 (m, 1H), 5.02-4.99 (m, 1H), 4.56 (s, 2H), 4.33 (s, 2H), 4.26-4.19 (m, 2H), 4.07-4.02 (m, 3H), 3.77-3.73 (m, 2H), 3.60-3.58 (m, 4H), 3.49 (s, 2H), 3.21-3.19 (m, 4H), 1.31 (q, J=9.2 Hz, 3H).; ESIMS: m/z=598.2 [(M+H)⁺].

### EXAMPLE 37

### Synthesis of 4-(4-((((2s,5s)-2-((1H-imidazol-1-yl)methyl)-2-(2-chlorophenyl)-1,3-dioxan-5-yl)oxy)methyl)phenyl)-N,N-dimethylpiperazine-1-carboxamide

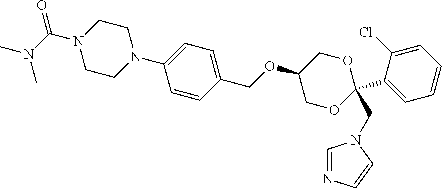 [SMILES: CN(C)C(=O)N1CCN(c2ccc(CO[C@H]3CO[C@](Cn4ccnc4)(c4ccccc4Cl)OC3)cc2)CC1]

The title compound was prepared according to the procedure for 4-(4-((((2s,5s)-2-((1H-imidazol-1-yl)methyl)-2-(2-chlorophenyl)-1,3-dioxan-5-yl)oxy)methyl)phenyl)morpholine, except N,N-dimethylpiperazine-1-carboxamide was substituted for morpholine. (400 MHz, CDCl₃) δ 7.43 (d, J=7.6 Hz, 1H), 7.29-7.26 (m, 2H), 7.25-7.23 (m, 1H), 7.20 (d, J=8.0 Hz, 1H), 7.14 (d, J=8.4 Hz, 2H), 6.90-6.85 (m, 3H), 6.72 (s, 1H), 4.39 (s, 3H), 4.19 (s, 2H), 4.01-3.96 (m, 2H), 3.74-3.64 (m, 1H), 3.39-3.36 (m, 6H), 3.19-3.15 (m, 4H), 2.86 (s, 6H); ESIMS: m/z=540.1 [(M+H)⁺].

### EXAMPLE 38

### Synthesis of 4-(4-((((2s,5s)-2-((1H-imidazol-1-yl)methyl)-2-(2-chlorophenyl)-1,3-dioxan-5-yl)oxy)methyl)phenyl)pyridine

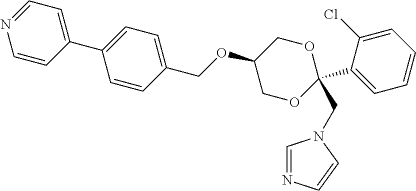 [SMILES: Clc1ccccc1[C@]1(Cn2ccnc2)OC[C@@H](OCc2ccc(-c3ccncc3)cc2)CO1]

To a stirred solution of 1-(((2s,5s)-5-((4-bromobenzyl)oxy)-2-(2-chlorophenyl)-1,3-dioxan-2-yl)methyl)-1H-imidazole (0.25 g, 0.4 mmol) in 1,4-Dioxane/water (9:1, 10 mL) was added K₂CO₃ (0.16 g, 1.2 mmol), pyridine-4-boronicacid pinacol ester (0.14 g, 0.7 mmol) and the reaction mixture was degassed with nitrogen gas for 10 minutes. Pd(dppf)Cl₂ (0.029 g, 0.04 mmol) was added and again degassed with nitrogen gas for 10 minutes. The reaction mixture was then refluxed for 12 hours. The reaction mixture was filtered and concentrated and the residue was purified by prep TLC to afford the title compound. ¹H NMR: (400 MHz, CDCl₃) δ 8.65 (d, J=5.2 Hz, 1H), 7.60 (d, J=8.4 Hz, 2H), 7.51-7.43 (m, 3H), 7.36 (d, J=8.4 Hz, 2H), 7.31-7.26 (m, 3H), 7.23-7.19 (m, 1H), 6.90 (s, 1H), 6.74 (s, 1H), 4.54 (s, 2H), 4.20 (s, 2H), 4.08-4.04 (m, 2H), 3.76-3.69 (m, 1H), 3.49-3.45 (m, 2H); ESIMS: m/z=462.1 [(M+H)⁺].

### EXAMPLE 39

The compounds in the Table 23 below represent the extraction of over 200 compounds which realized the in vitro and in vivo goals. The target goals are defined in Table 22. As seen in Table 22 below, the in vitro goals are defined by efficacy targets: CYP17, CYP11, and CYP21. The off-target enzymes (where potency should be low) are CYP19 and CYP3A4. Other parameters are no liver effects also estimated by bile acid synthesis inhibition.

TABLE 22
In vitro goals for compounds.
Product Profile
Target Activity

| | |
|---|---|
| Cyp17 | <100 nM* |
| Cyp21 | <100 nM* |
| Cyp11B1 | <100 nM* |

Off Target Counter Screens

| | |
|---|---|
| Cyp19 | 50x Cyp 17 |
| Bile Acid (Cyp7A surrogate) | 100 x Cyp 17 |
| Cyp450 3A4 >50% @ 1 mM | IC₅₀ (nM) |
| Cyp450 2D6 >50% @ 1 mM | >50% @ 1 μM |
| Cyp450 2C9 >50% @ 1 mM | >50% @ 1 μM |

Pharmaceutical Profiling/ADME

| | |
|---|---|
| Solubility >5 uMolar | uMolar |
| Molecular Weight | <500 AMU |
| ClogP | <5 |
| TPSA | <140 |
| Cytoxicity in HerG2 | >10% @ 1 μM |
| Caco-2 permeability (A-B, B-A) | >20 (10⁻⁶ cm/sec) |
| Guinea Pig liver microsomal stability | >30 min. |
| Human liver microsomal stability | >30 min. |
| Plasma stability | >30 min. |
| Plasma protein binding | <95% |
| Guinea Pig Bioavailability | >25% |
| Guinea Pig T_{1/2} | <4 h |

TABLE 23
Representative examples of compounds of the disclosure and their potencies in Cyp17, Cyp11, and Cyp21 assays.

| | | | | | | | | | Bile Acid |
|---|---|---|---|---|---|---|---|---|---|
| | | | | Cyp17 | Cyp11 | Cyp21 | Cyp19 | Cyp3A4 | Secretion |
| Structure | MW | TPSA | LogP | IC₅₀ (nM) | | | | | |
| 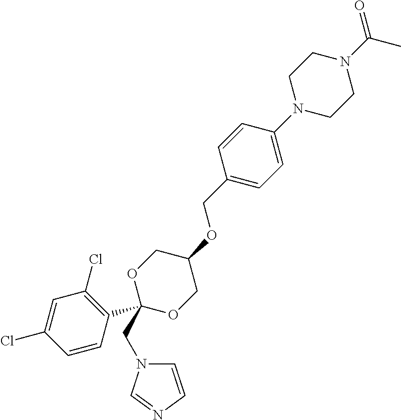 [SMILES: CC(=O)N1CCN(c2ccc(CO[C@H]3CO[C@](Cn4ccnc4)(c4ccc(Cl)cc4Cl)OC3)cc2)CC1] | 545 | 69 | 4.2 | 8 | 12 | 208 | 350 | 109 | 880 |

### EXAMPLE 40

Pharmacokinetic studies in the guinea pig were run using 1 mg/kg IV dosing (20% DMA, 40% TEG, 40% water) and 10 mg/kg oral dosing (2% Tween-80, 98% HPMC (1% water)). The oral PK data is summarized in the tables below.

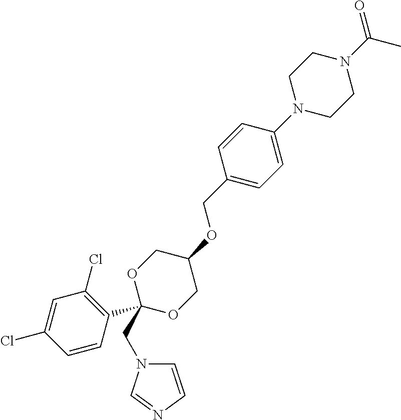 [SMILES: CC(=O)N1CCN(c2ccc(CO[C@H]3CO[C@](Cn4ccnc4)(c4ccc(Cl)cc4Cl)OC3)cc2)CC1]

| | |
|---|---|
| Dose (mg/kg) | 10 |
| Cmax (ng/mL) | 1018 |
| Tmax | 3.00 |
| t1/2 (h) | 6.0 |
| AUC₀₋ₗₐₛₜ | 8231 |
| (ng · h/mL) | |
| AUC_{0-inf} | 14891 |
| (ng · h/mL) | |
| AUC_{Extra} (%) | 43.93 |
| DNAUC (0-inf) | 823.1 |
| MRT₀₋ₗₐₛₜ (h) | 5.56 |
| Rsq | 0.91 |
| Bioavailability | 184.21 |

### Formulations

Some embodiments of the present invention also relates to compositions or formulations which comprise the cortisol lowering agents according to embodiments described herein. In general, the compositions of embodiments described herein comprise an effective amount of one or more compounds of the disclosure and salts thereof according to embodiments described herein which are effective for providing cortisol lowering; and one or more excipients.

In this document, the term “excipient” and “carrier” are used interchangeably and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means to achieve effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of embodiments described herein have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

The compounds of embodiments described herein can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of embodiments described herein (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of embodiments described herein and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of embodiments described herein. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of embodiments described herein can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to embodiments described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of embodiments described herein can also be formulated in rectal compositions such as suppositories or retention enemas, e.g., containing conventional suppository bases such as cocoa butter or other glycerides.

In addition to the formulations described previously, the compounds of embodiments described herein can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of embodiments described herein, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of embodiments described herein can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, poly ethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to embodiments described herein include from about 0.001 mg to about 1000 mg of one or more compounds of the disclosure according to embodiments described herein and one or more excipients; from about 0.01 mg to about 100 mg of one or more compounds of the disclosure according to embodiments described herein and one or more excipients; from about 100 mg to about 250 mg of one or more compounds of the disclosure according to embodiments described herein and one or more excipients; from about 250 mg to about 500 mg of one or more compounds of the disclosure according to embodiments described herein and one or more excipients; from about 500 mg to about 750 mg of one or more compounds of the disclosure according to embodiments described herein and one or more excipients; from about 750 mg to about 1000 mg of one or more compounds of the disclosure according to embodiments described herein and one or more excipients; and from about 0.1 mg to about 10 mg of one or more compounds of the disclosure according to embodiments described herein; and one or more excipients.

In some embodiments, the compositions according to embodiments described herein are administered orally to a patient once daily.

In some embodiments, the compositions according to embodiments described herein are administered orally to a patient twice daily.

In some embodiments, the compositions according to embodiments described herein are administered orally to a patient three time per day.

In some embodiments, the compositions according to embodiments described herein are administered orally to a patient once weekly.

### Procedures

The following procedures can be utilized in evaluating and selecting compounds as cortisol lowering agents.

Cyp17 assay protocol: AD293 cells that stably over-express recombinant CYP-17 were seeded in 96 well plates coated with poly D-lysine (15,000 cell per well) and incubated at 37° C. for 24 hours in Dulbecco's Modified Eagle Medium (DMEM) with Fetal Bovine Serum (FBS) that is stripped of hormones by charcoal treatment. The media is then removed, the cells are washed once with Phosphate buffer saline solution, and 50 μL Dulbecco's Modified Eagle Medium (DMEM) with Fetal Bovine Serum (FBS) that is stripped of hormones by charcoal treatment is added. Compounds of the disclosure are then added to the wells in eight concentration spanning 10 μM to 4.5 nM, and the plates are incubated for an additional 60 minutes at 37° C. [21-³H] 17α-hydroxyl-Pregnenolone is then added (50 nCi per well, 31.25 nM) and the plates are incubated for an additional 4 hours at 37° C. The media is then collected, 200 μL, of chloroform is added, and the mixture is shaken for 1 hour. The aqueous layer is then separated and analyzed for the presence of ³H-acetic acid using a Perkin Elmer Topcount NXT to determine IC₅₀s of the compounds of the disclosure.

Cyp21 assay protocol: AD293 cells that stably over-express recombinant CYP-21 were seeded in 96 well plates coated with poly D-lysine (10,000 cell per well) and incubated at 37° C. for 24 hours in Dulbecco's Modified Eagle Medium (DMEM) with Fetal Bovine Serum (FBS) that is stripped of hormones by charcoal treatment. The media is then removed, the cells are washed once with Phosphate buffer saline solution, and 50 μL Dulbecco's Modified Eagle Medium (DMEM) with Fetal Bovine Serum (FBS) that is stripped of hormones by charcoal treatment is added. Compounds of the disclosure are then added to the wells in eight concentration spanning 10 μM to 4.5 nM, and the plates are incubated for an additional 60 minutes at 37° C. 17α-OH Progesterone is then added (1.0 μM) and the plates are incubated for an additional 45 minutes at 37° C. After incubation, 50 uL of the supernatant (medium) is transferred into a fresh plate and 150 uL of an acetonitrile solution containing 200 ng/ml of Telmisartan is added. The sample is mixed and then placed in a centrifuge at 2000 rpm for 5 minutes. 100 uL of the supernatant is transferred into a fresh 96 well deep well plate, 100 uL of 1:1 methanol:water was added, the solution was mixed and then analyzed by LC/MS for the presence of 11-deoxycortisol using an Agilent 1200 RRLC/ABSCIEX API4000 LC-MS or Shimadzu Prominance/ABSCIEX API4000 LC-MS to determine IC₅ₒs of the compounds of the disclosure.

Cyp11 assay protocol: AD293 cells that stably over-express recombinant CYP-11 were seeded in 96 well plates coated with poly D-lysine (15,000 cell per well) and incubated at 37° C. for 24 hours in Dulbecco's Modified Eagle Medium (DMEM) with Fetal Bovine Serum (FBS) that is stripped of hormones by charcoal treatment. The media is then removed, the cells are washed once with Phosphate buffer saline solution, and 50 μL Dulbecco's Modified Eagle Medium (DMEM) with Fetal Bovine Serum (FBS) that is stripped of hormones by charcoal treatment is added. Compounds of the disclosure are then added to the wells in eight concentration spanning 10 μM to 4.5 nM, and the plates are incubated for an additional 60 minutes at 37° C. 11-deoxycortisol is then added (2.0 μM) and the plates are incubated for an additional 12 hours at 37° C. After incubation, 50 uL of the supernatant (medium) is transferred into a fresh plate and 150 uL of an acetonitrile solution containing 200 ng/ml of Telmisartan is added. The sample is mixed and then placed in a centrifuge at 2000 rpm for 5 minutes. 100 uL of the supernatant is transferred into a fresh 96 well deep well plate, 100 uL of 1:1 methanol:water was added, the solution was mixed and then analyzed by LC/MS for the presence of cortisol using an Agilent 1200 RRLC/ABSCIEX API4000 LC-MS or Shimadzu Prominance/ABSCIEX API4000 LC-MS to determine IC₅ₒs of the compounds of the disclosure.

Results for representative compounds according to embodiments described herein are listed in Table 23.

TABLE 23
Representative examples of compounds of the disclosure and their potencies in Cyp17, Cyp11, and Cyp21 assays.

| | | | Cyp17 | Cyp11 | Cyp21 |
|---|---|---|---|---|---|
| | | | IC₅₀ | IC₅₀ | IC₅₀ |
| | Entry | Structure | (nM) | | |
| 1 | 500003 | 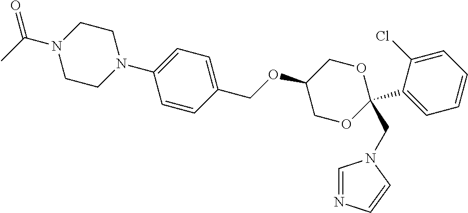 [SMILES: CC(=O)N1CCN(c2ccc(CO[C@H]3CO[C@](Cn4ccnc4)(c4ccccc4Cl)OC3)cc2)CC1] | 146 | 521 | 1480 |
| 2 | 500004 | 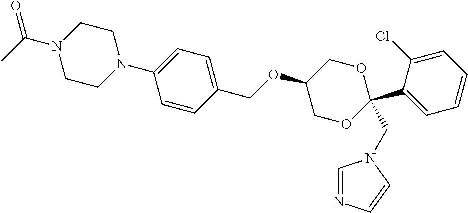 [SMILES: CC(=O)N1CCN(c2ccc(CO[C@H]3CO[C@@](Cn4ccnc4)(c4ccccc4Cl)OC3)cc2)CC1] | 10000 | | 10000 |
| 3 | 500005 | 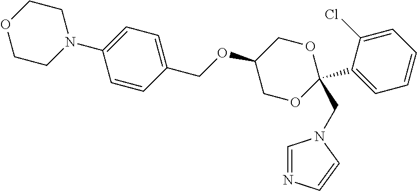 [SMILES: Clc1ccccc1[C@]1(Cn2ccnc2)OC[C@@H](OCc2ccc(N3CCOCC3)cc2)CO1] | 260 | | 960 |
| 4 | 500006 | 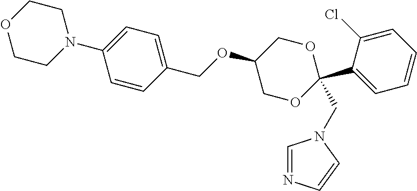 [SMILES: Clc1ccccc1[C@]1(Cn2ccnc2)OC[C@H](OCc2ccc(N3CCOCC3)cc2)CO1] | 8000 | | 4760 |
| 5 | 500012 | 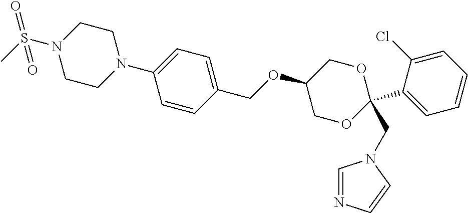 [SMILES: CS(=O)(=O)N1CCN(c2ccc(CO[C@H]3CO[C@](Cn4ccnc4)(c4ccccc4Cl)OC3)cc2)CC1] | 12 | 100 | 1520 |
| 6 | 500013 | 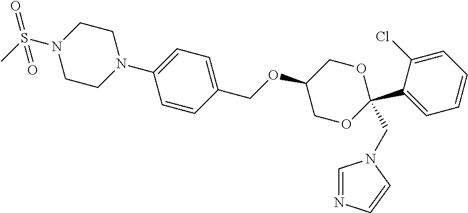 [SMILES: CS(=O)(=O)N1CCN(c2ccc(CO[C@H]3CO[C@@](Cn4ccnc4)(c4ccccc4Cl)OC3)cc2)CC1] | 1500 | 10000 | 2500 |
| 7 | 510013 | 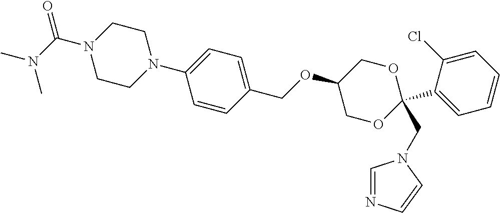 [SMILES: CN(C)C(=O)N1CCN(c2ccc(CO[C@H]3CO[C@](Cn4ccnc4)(c4ccccc4Cl)OC3)cc2)CC1] | 27 | 70 | 800 |
| 8 | 510012 | 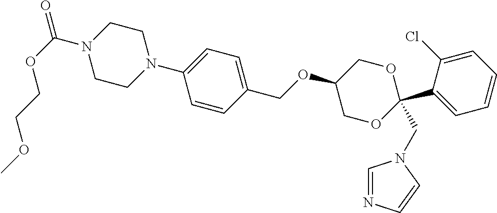 [SMILES: COCCOC(=O)N1CCN(c2ccc(CO[C@H]3CO[C@@](Cn4ccnc4)(c4ccccc4Cl)OC3)cc2)CC1] | 2500 | | 800 |
| 9 | 510014 | 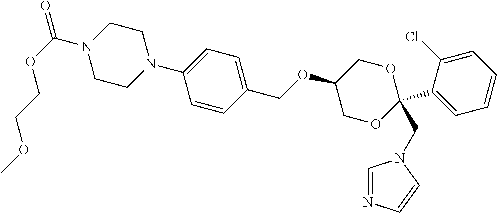 [SMILES: COCCOC(=O)N1CCN(c2ccc(CO[C@H]3CO[C@](Cn4ccnc4)(c4ccccc4Cl)OC3)cc2)CC1] | 20 | 140 | 570 |
| 10 | 510017 | 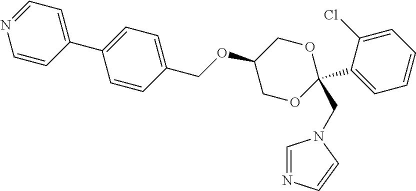 [SMILES: Clc1ccccc1[C@]1(Cn2ccnc2)OC[C@@H](OCc2ccc(-c3ccncc3)cc2)CO1] | 42 | 440 | 391 |
| 11 | 510018 | 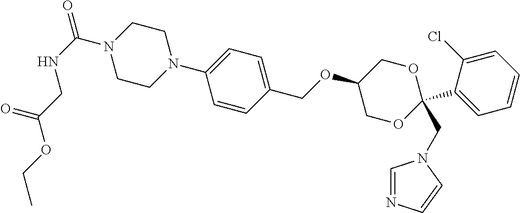 [SMILES: CCOC(=O)CNC(=O)N1CCN(c2ccc(CO[C@H]3CO[C@](Cn4ccnc4)(c4ccccc4Cl)OC3)cc2)CC1] | 40 | 107 | 2800 |
| 12 | 510019 | 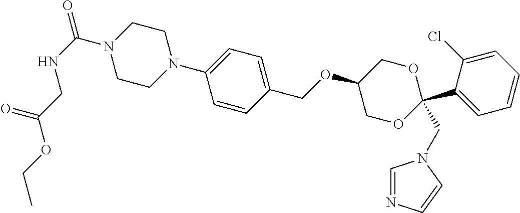 [SMILES: CCOC(=O)CNC(=O)N1CCN(c2ccc(CO[C@H]3CO[C@@](Cn4ccnc4)(c4ccccc4Cl)OC3)cc2)CC1] | 10000 | 5800 | 10000 |
| 13 | 500015 | 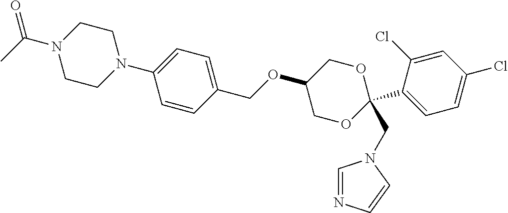 | 8 | 12 | 208 |
| 14 | 500016 | 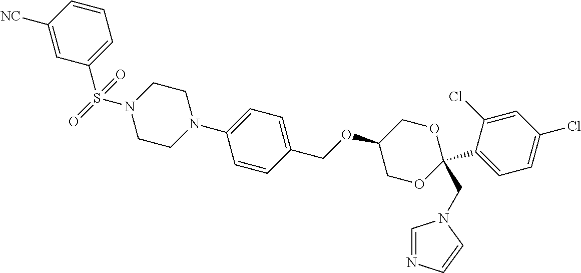 [SMILES: N#Cc1cccc(S(=O)(=O)N2CCN(c3ccc(CO[C@H]4CO[C@](Cn5ccnc5)(c5ccc(Cl)cc5Cl)OC4)cc3)CC2)c1] | 75 | 37 | 415 |
| 15 | 500017 | 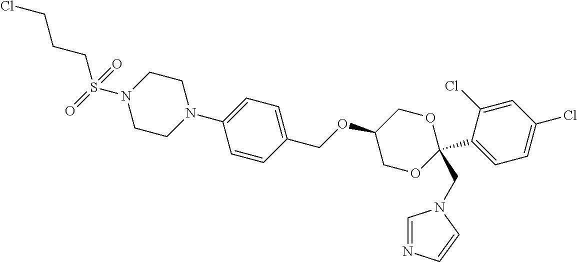 [SMILES: O=S(=O)(CCCCl)N1CCN(c2ccc(CO[C@H]3CO[C@](Cn4ccnc4)(c4ccc(Cl)cc4Cl)OC3)cc2)CC1] | 106 | 27 | 1100 |
| 16 | 500018 | 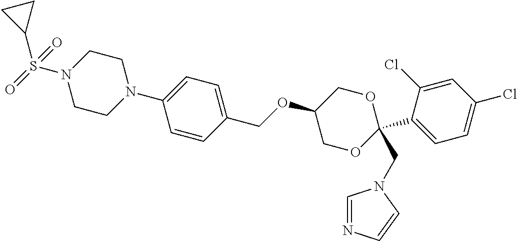 [SMILES: O=S(=O)(C1CC1)N1CCN(c2ccc(CO[C@H]3CO[C@](Cn4ccnc4)(c4ccc(Cl)cc4Cl)OC3)cc2)CC1] | 11 | 6 | 262 |
| 17 | 500019 | 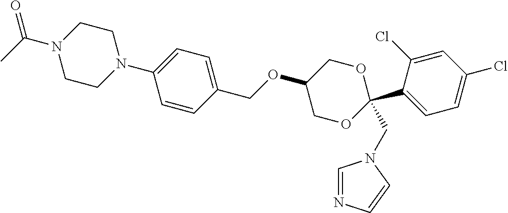 | 1100 | 12 | 10000 |
| 18 | 500020 | 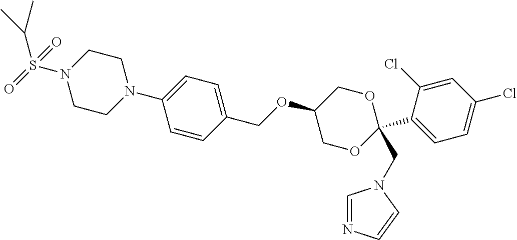 [SMILES: CC(C)S(=O)(=O)N1CCN(c2ccc(CO[C@H]3CO[C@](Cn4ccnc4)(c4ccc(Cl)cc4Cl)OC3)cc2)CC1] | 11 | 25 | 110 |
| 19 | 500021 | 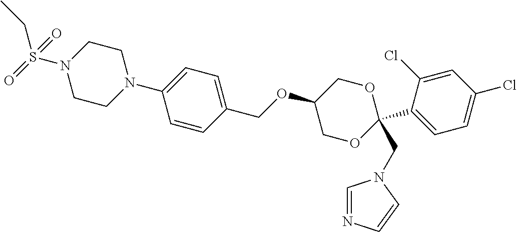 [SMILES: CCS(=O)(=O)N1CCN(c2ccc(CO[C@H]3CO[C@](Cn4ccnc4)(c4ccc(Cl)cc4Cl)OC3)cc2)CC1] | 9 | 200 | 130 |
| 20 | 500024 | 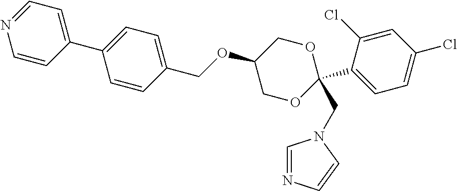 [SMILES: Clc1ccc([C@]2(Cn3ccnc3)OC[C@@H](OCc3ccc(-c4ccncc4)cc3)CO2)c(Cl)c1] | 33 | 23 | 68 |
| 21 | 500025 | 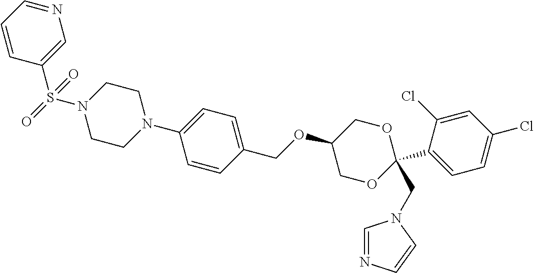 [SMILES: O=S(=O)(c1cccnc1)N1CCN(c2ccc(CO[C@H]3CO[C@](Cn4ccnc4)(c4ccc(Cl)cc4Cl)OC3)cc2)CC1] | 13 | 12 | 273 |
| 22 | 500022 | 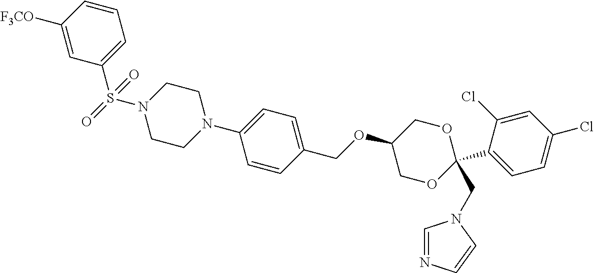 [SMILES: O=S(=O)(c1cccc(OC(F)(F)F)c1)N1CCN(c2ccc(CO[C@H]3CO[C@](Cn4ccnc4)(c4ccc(Cl)cc4Cl)OC3)cc2)CC1] | 101 | 13 | 170 |
| 23 | 500023 | 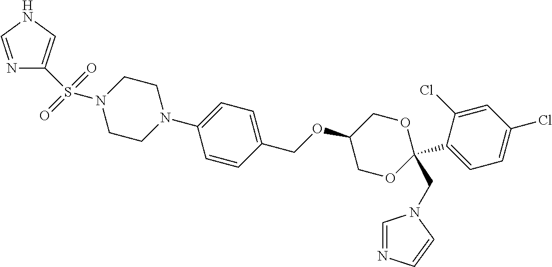 [SMILES: O=S(=O)(c1c[nH]cn1)N1CCN(c2ccc(CO[C@H]3CO[C@](Cn4ccnc4)(c4ccc(Cl)cc4Cl)OC3)cc2)CC1] | 67 | 14 | 426 |

1-34. (canceled)

35. A compound comprising formula (I): 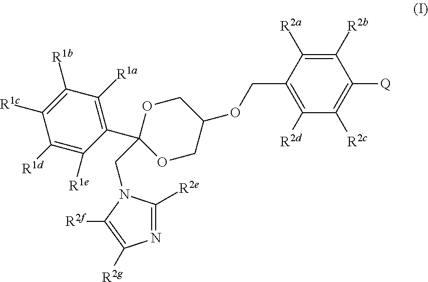
or a hydrate, solvate, enantiomer, diastereomer, pharmaceutically acceptable salt, prodrug or complex thereof, wherein
Q is selected from the group consisting of optionally substituted aryl, optionally substituted heteroaryl, 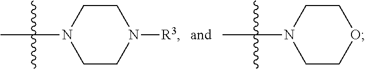
R^{1a}, R^{1b}, R^{1c}, R^{1d}, and R^{1e} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;
R^{2a}, R^{2b}, R^{2c}, R^{2d}, R^{2e}, R^{2f} and R^{2g} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆ optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;
R³ is selected from a group consisting of —SO₂R⁸, —C(O)NR⁹R¹⁰, —C(O)OR⁷, 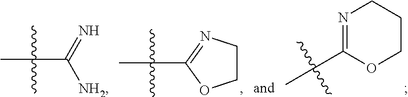
R^{4a} and R^{4b} are each independently selected from the group consisting of hydrogen, optionally substituted C₁₋₆ linear alkyl, optionally substituted C₁₋₆ branched alkyl, and optionally substituted C₃₋₇ cycloalkyl;
R⁵ is selected from the group consisting of hydrogen, optionally substituted C₁₋₆ linear alkyl, optionally substituted C₁₋₆ branched alkyl, and optionally substituted C₃₋₇ cycloalkyl;
R⁶ is selected from the group consisting of hydrogen, optionally substituted C₁₋₆ linear alkyl, optionally substituted C₁₋₆ branched alkyl, and optionally substituted C₃₋₇ cycloalkyl;
R⁷ is selected from the group consisting of optionally substituted C₁₋₆ linear alkyl, optionally substituted C₁₋₆ branched alkyl, and optionally substituted C₃₋₇ cycloalkyl;
R⁸ is selected from the group consisting of optionally substituted C₁₋₆ linear alkyl, optionally substituted C₁₋₆ branched alkyl, optionally substituted C₃₋₇ cycloalkyl, optionally substituted C₁₋₆ haloalkyl, optionally substituted aryl, optionally substituted heteroaryl, and optionally substituted C3-7 heterocyclyl;
R⁹ is selected from the group consisting of hydrogen, optionally substituted C₁₋₆ linear alkyl, optionally substituted C₁₋₆ branched alkyl, optionally substituted C₃₋₇ cycloalkyl, optionally substituted C₁₋₆ haloalkyl, 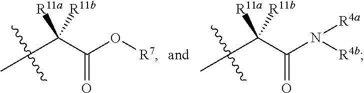
R¹⁰ is selected from the group consisting of hydrogen, optionally substituted C₁₋₆ linear alkyl, and optionally substituted C₁₋₆ branched alkyl; and
R^{11a} and R^{11b} are each independently selected from the group consisting of hydrogen, optionally substituted C₁₋₆ linear alkyl, optionally substituted C₁₋₆ branched alkyl, optionally substituted aryl, optionally substituted benzyl, —CH₂OR⁶, and CH₂ heteroaryl.

36. The compound of claim 35, wherein the compound is selected from the group consisting of formula (II), formula (III), formula (IV), formula (V), formula (VI), formula (VII), formula (VIII), formula (IX), formula (X), formula (Xa), and any hydrate, solvate, enantiomer, disastereomer, pharmaceutically acceptable salt, prodrug, or complex thereof.

37. The compound of claim 35, wherein the compound is selected from the group consisting of:
1-(4-(4-((((2s,5s)-2-((1H-imidazol-1-yl)methyl)-2-(2,4-dichlorophenyl)-1,3-dioxan-5-yl)oxy)methyl)phenyl)piperazin-1-yl)ethanone;
1-(4-(4-((((2r,5r)-2-((1H-imidazol-1-yl)methyl)-2-(2,4-dichlorophenyl)-1,3-dioxan-5-yl)oxy)methyl)phenyl)piperazin-1-yl)ethanone;
1-(4-((((2s,5s)-2-((1H-imidazol-1-yl)methyl)-2-(2,4-dichlorophenyl)-1,3-dioxan-5-yl)oxy)methyl)phenyl)piperazine:
3-((4-(4-((((2s,5s)-2-((1H-imidazol-1-yl)methyl)-2-(2,4-dichlorophenyl)-1,3-dioxan-5-yl)oxy)methyl)phenyl)piperazin-1-yl)sulfonyl)benzonitrile;
1-(4-((((2s,5s)-2-((1H-imidazol-1-yl)methyl)-2-(2,4-dichlorophenyl)-1,3-dioxan-5-yl)oxy)methyl)phenyl)-4-((3-chloropropyl)sulfonyl)piperazine;
1-(4-((((2s,5s)-2-((1H-imidazol-1-yl)methyl)-2-(2,4-dichlorophenyl)-1,3-dioxan-5-yl)oxy)methyl)phenyl)-4-(cyclopropylsulfonyl)piperazine;
1-(4-((((2s,5s)-2-((1H-imidazol-1-yl)methyl)-2-(2,4-dichlorophenyl)-1,3-dioxan-5-yl)oxy)methyl)phenyl)-4-(isopropylsulfonyl)piperazine;
1-(4-((((2s,5s)-2-((1H-imidazol-1-yl)methyl)-2-(2,4-dichlorophenyl)-1,3-dioxan-5-yl)oxy)methyl)phenyl)-4-(ethylsulfonyl)piperazine;
1-(4-((((2s,5s)-2-((1H-imidazol-1-yl)methyl)-2-(2,4-dichlorophenyl)-1,3-dioxan-5-yl)oxy)methyl)phenyl)-4-((1H-imidazol-4-yl)sulfonyl)piperazine;
1-(4-((((2s,5s)-2-((1H-imidazol-1-yl)methyl)-2-(2,4-dichlorophenyl)-1,3-dioxan-5-yl)oxy)methyl)phenyl)-4-((3-(trifluoromethoxy)phenyl)sulfonyl)piperazine;
1-(4-((((2s,5s)-2-((1H-imidazol-1-yl)methyl)-2-(2,4-dichlorophenyl)-1,3-dioxan-5-yl)oxy)methyl)phenyl)-4-(pyridin-3-ylsulfonyl)piperazine;
1-(4-(4-((((2s,5s)-2-((1H-imidazol-1-yl)methyl)-2-(2-chlorophenyl)-1,3-dioxan-5-yl)oxy)methyl)phenyl)piperazin-1-yl)ethanone;
1-(4-(4-((((2r,5r)-2-((1H-imidazol-1-yl)methyl)-2-(2-chlorophenyl)-1,3-dioxan-5-yl)oxy)methyl)phenyl)piperazin-1-yl)ethanone;
4-(4-((((2s,5s)-2-((1H-imidazol-1-yl)methyl)-2-(2,4-dichlorophenyl)-1,3-dioxan-5-yl)oxy)methyl)phenyl)pyridine;
4-(4-((((2s,5s)-2-((1H-imidazol-1-yl)methyl)-2-(2-chlorophenyl)-1,3-dioxan-5-yl)oxy)methyl)phenyl)morpholine;
4-(4-((((2r,5r)-2-((1H-imidazol-1-yl)methyl)-2-(2-chlorophenyl)-1,3-dioxan-5-yl)oxy)methyl)phenyl)morpholine;
1-(4-((((2s,5s)-2-((1H-imidazol-1-yl)methyl)-2-(2-chlorophenyl)-1,3-dioxan-5-yl)oxy)methyl)phenyl)-4-(methylsulfonyl)piperazine;
1-(4-((((2r,5r)-2-((1H-imidazol-1-yl)methyl)-2-(2-chlorophenyl)-1,3-dioxan-5-yl)oxy)methyl)phenyl)-4-(methylsulfonyl)piperazine;
2-methoxyethyl 4-(4-((((2s,5s)-2-((1H-imidazol-1-yl)methyl)-2-(2-chlorophenyl)-1,3-dioxan-5-yl)oxy)methyl)phenyl)piperazine-1-carboxylate;
2-methoxyethyl 4-(4-((((2r,5r)-2-((1H-imidazol-1-yl)methyl)-2-(2-chlorophenyl)-1,3-dioxan-5-yl)oxy)methyl)phenyl)piperazine-1-carboxylate;
ethyl 2-(4-(4-((((2s,5s)-2-((1H-imidazol-1-yl)methyl)-2-(2-chlorophenyl)-1,3-dioxan-5-yl)oxy)methyl)phenyl)piperazine-1-carboxamido)acetate;
ethyl 2-(4-(4-((((2r,5r)-2-((1H-imidazol-1-yl)methyl)-2-(2-chlorophenyl)-1,3-dioxan-5-yl)oxy)methyl)phenyl)piperazine-1-carboxamido)acetate;
4-(4-((((2s,5s)-2-((1H-imidazol-1-yl)methyl)-2-(2-chlorophenyl)-1,3-dioxan-5-yl)oxy)methyl)phenyl)-N,N-dimethylpiperazine-1-carboxamide;
4-(4-((((2s,5s)-2-((1H-imidazol-1-yl)methyl)-2-(2-chlorophenyl)-1,3-dioxan-5-yl)oxy)methyl)phenyl)pyridine;
and a pharmaceutically acceptable salt form thereof.

38. The compound of claim 35 included in an effective amount in a composition.

39. The compound of claim 38 wherein the composition comprises at least one excipient.

40. A compound selected from the group consisting of formula (X), formula (Xa) and any hydrate, solvate, enantiomer, diastereomer, pharmaceutically acceptable salt, prodrug or complex thereof, wherein
R^{1a}, R^{1b}, R^{1c}, R^{1d}, and R^{1e} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆, optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;
R^{2a}, R^{2b}, R^{2c}, R^{2d}, R^{2e}, R^{2f} and R^{2g} are each independently selected from the group consisting of hydrogen, halogen, OH, optionally substituted C₁₋₆ linear alkyl, optionally substituted C₁₋₆ branched alkyl, optionally substituted C₃₋₇ cycloalkyl, optionally substituted C₁₋₆ haloalkyl, C₁₋₆ optionally substituted alkoxy, —NR^{4a}R^{4b}, —NR⁵COR⁶, —CO₂R⁶, —CO₂NR^{4a}R^{4b}, —NHSO₂R⁷, —SH, —SR⁷, SO₂R⁷ and —SO₂NHR⁶;
R^{4a} and R^{4b} are each independently selected from the group consisting of hydrogen, optionally substituted C₁₋₆ linear alkyl, optionally substituted C₁₋₆ branched alkyl, and optionally substituted C₃₋₇ cycloalkyl;
R⁵ is selected from the group consisting of hydrogen, optionally substituted C₁₋₆ linear alkyl, optionally substituted C₁₋₆ branched alkyl, and optionally substituted C₃₋₇ cycloalkyl;
R⁶ is selected from the group consisting of hydrogen, optionally substituted C₁₋₆ linear alkyl, optionally substituted C₁₋₆ branched alkyl, and optionally substituted C₃₋₇ cycloalkyl; and
R⁷ is selected from the group consisting of optionally substituted C₁₋₆ linear alkyl, optionally substituted C₁₋₆branched alkyl, and optionally substituted C₃₋₇ cycloalkyl.

41. The compound of claim 40 included in an effective amount in a composition.

42. The compound of claim 41 wherein the composition comprises at least one excipient.

43. A method of treating a disease associated with overproduction of cortisol, said method comprising administering to a subject having the disease an effective amount of a pharmaceutical composition comprising a compound of claim 35.

44. The method of claim 43, wherein the disease associated with overproduction of cortisol is metabolic syndrome, obesity, headache, depression, hypertension, diabetes mellitus, Cushing's Syndrome, pseudo-Cushing syndrome, cognitive impairment, dementia, heart failure, renal failure, psoriasis, glaucoma, cardiovascular disease, stroke or incidentalomas.

45. The method of claim 43, wherein the disease is also associated with excess Cyp17 activity.

46. The method of claim 43, wherein the disease is also associated with excess Cyp21 activity.

47. The method of claim 43, wherein the disease is also associated with excess Cyp11B1 activity.

48. The method of claim 43, wherein the pharmaceutical composition comprises at least one excipient.
